(19) United States
(12) Patent Application Publication
(10) Pub. No.: US 2022/0372447 A1
(43) Pub. Date: Nov. 24, 2022

(54) GENERATION OF INDUCED PLURIPOTENT STEM CELLS WITH POLYCISTRONIC SOX2, KLF4, AND OPTIONALLY C-MYC

(71) Applicant: The J. David Gladstone Institutes, a testamentary trust established under the Will of J. David Glads, San Francisco, CA (US)

(72) Inventors: Peng LIU, San Francisco, CA (US); Sheng DING, Orinda, CA (US)

(21) Appl. No.: 17/769,865

(22) PCT Filed: Oct. 16, 2020
(86) PCT No.: PCT/US2020/055943

§ 371 Date: Apr. 18, 2022

Related U.S. Application Data
(60) Provisional application No. 62/916,830, filed on Oct. 18, 2019.

(51) Int. Cl. C12N 5/074

(52) U.S. Cl. CPC C12N 5/0696; C12N 2740/16043; C12N 2501/604; C12N 2501/602; C12N 2501/606; C12N 2502/13

(57) ABSTRACT

Described herein a polycistronic expression cassettes and expression vectors that include a promoter operably linked to a nucleic acid segment that encodes a Sox2 and Klf4 polypeptide. The nucleic acid segment can also encode a c-Myc polypeptide. Expression of such polycistronic expression cassettes/vectors in host cells can reprogram the host cells to stem cells or other types of reprogrammed cells.

This application claims benefit of priority to the filing date of U.S. Provisional Application Ser. No. 62/916,830, filed Oct. 18, 2019, the contents of which are specifically incorporated herein by reference in their entirety.

### INCORPORATION BY REFERENCE OF SEQUENCE LISTING PROVIDED AS A TEXT FILE

A Sequence Listing is provided herewith as a text file, “373038WOSEQ LIST.txt” created on Oct. 16, 2020 and having a size of 53,248 bytes. The contents of the text file are incorporated by reference herein in their entirety.

### BACKGROUND

The first demonstration that differentiated somatic cells can be reprogrammed into induced pluripotent stem cells (iPSCs) utilized ectopic expression of four factors: Oct4 (O), Sox2 (S), Klf4 (K), and c-Myc (M) (Takahashi and Yamanaka, 2006). For many years, Oct4 has been considered indispensable in the reprogramming process, because it is the only one of those four that is sufficient to induce pluripotency alone and its family members cannot replace its function (Kim et al., 2009a; Kim et al., 2009b; Nakagawa et al., 2008). Mechanistic investigations have shown that reprogramming is initiated by the global cooperative engagement of three pioneer factors Oct4, Sox2, and Klf4, followed by genome-wide epigenetic remodeling and two transcriptional waves (Chen et al., 2016; Chronis et al., 2017; Polo et al., 2012; Smith et al., 2016; Soufi et al., 2012; Sridharan et al., 2009). These studies emphasize the cooperative effect of Oct4, Sox2 and Klf4 (Chronis et al., 2017; Sridharan et al., 2009) but do not explain why Oct4 is unique, and the function of Sox2 and Klf4 in this process remains underappreciated.

### SUMMARY

Methods and compositions are described here for precisely controlling factor stoichiometry during cellular reprogramming by using polycistronic cassettes. Surprisingly, the data described herein show that in the absence of ectopic Oct4, polycistronic Sox2, Klf4, and c-Myc (referred to, for example, as the S_{2A}K_{2A}M polycistronic construct) was sufficient to establish pluripotency in several types of differentiated somatic cells. In some cases, c-Myc was optional and use of polycistronic Sox2 and Klf4 (for example, S_{2A}K) was sufficient. The stoichiometry of Sox2 and Klf4 was more important for this reprogramming (e.g., than that of c-Myc), as disruption of the Sox2 and Klf4 factor balance led to a significant decrease or failure in iPSC generation. Genome wide investigations revealed cooperative binding of Sox2 and Klf4, leading to gradual activation and establishment of pluripotency network. Moreover, parallel transcriptomic analysis with secondary S_{2A}K_{2A}M embryonic fibroblasts (2° MEFs) and neural progenitor cells (2° NPCs) demonstrated convergent reprogramming trajectories and similar efficiency. The results shown herein illustrate the stoichiometric sufficiency of Sox2 and Klf4 in pluripotency induction without ectopic Oct4. The data provided herein demonstrate the core functions of Sox2 and Klf4 in pluripotency induction.

### DESCRIPTION OF THE FIGURES

FIG. 1A-1P illustrate that the polycistronic S_{2A}K_{2A}M expression cassette (expressing Sox2, Klf4 and Myc-C with 2A cleavable linker between the Sox2 and Klf4 and between the Klf4 and Myc-C) reprograms mouse embryonic fibroblasts (MEFs) into induced pluripotent stem cells (iPSCs). FIG. 1A shows a schematic depicting a S_{2A}K_{2A}M polycistronic expression system and a reprogramming procedure. FIG. 1B shows images of colonies obtained from S_{2A}K_{2A}M reprogramming illustrating EGFP expression by the colonies on day 7 of reprogramming (scale bar, 100 μm). PH, phase contrast. The MEFs expressed Oct4-GFP (OG2 cells) as a marker of pluripotency, where the Oct4 promoter was operably linked to a segment encoding Enhanced Green Fluorescent Protein (EGFP). FIG. 1C shows images of S_{2A}K_{2A}M colonies illustrating the EGFP signal in situ and at passages 1 and 20 (scale bar, 100 μm). FIG. 1D illustrates that S_{2A}K_{2A}M induced pluripotent stem cells (iPSCs) showed complete DNA demethylation at the Oct4 promoter. FIG. 1E illustrates that Nanog, Sox2, and SSEA1 proteins were detected in S_{2A}K_{2A}M iPSCs (scale bar, 100 μm). FIG. 1F graphically illustrates a correlation of global gene expression in S_{2A}K_{2A}M iPSCs with R1 embryonic stem cells (ESCs). FIG. 1G shows images of chimeric mice generated by injection of S_{2A}K_{2A}M iPSCs into blastocysts that were implanted in pseudo-pregnant females, as confirmation that the S_{2A}K_{2A}M iPSCs were pluripotent. FIG. 1H shows mouse embryos formed by tetraploid complementation assay involving electrofusing cell-stage CD1 (ICR) embryos to produce tetraploid embryos, and injecting S_{2A}K_{2A}M iPSCs into the embryos to form reconstructed tetraploid blastocysts that were implanted into pseudo-pregnant CD1 (ICR) female mice. FIG. 1I illustrates that the S_{2A}K_{2A}M iPSCs contributed to germ cells in the implanted blastocysts. FIG. 1J shows schematics illustrating additional polycistronic cassettes for O_{2A}S_{2A}K_{2A}M, O_{2A}S_{2A}M, O_{2A}K_{2A}M, and S_{2A}K_{2A}M, where O refers to Oct4, S refers to Sox2, K refers to Klf4, and M refers to c-Myc. FIG. 1K shows western blots illustrating protein expression in MEFs from the O_{2A}S_{2A}M, O_{2A}K_{2A}M, and S_{2A}K_{2A}M expression cassettes when expression was induced for 48 hours. FIG. 1L shows western blots after long exposure illustrating efficient cleavage of the polycistronic polypeptide at the 2A sites in transduced MEFs. FIG. 1M-1 to 1M-4 graphically illustrate Oct4-EGFP colony numbers during a 14-day induction of O_{2A}S_{2A}K_{2A}M, O_{2A}S_{2A}M, O_{2A}K_{2A}M, and S_{2A}K_{2A}M in 100,000 starting OG2 MEFs. FIG. 1M-1 graphically illustrates Oct4-EGFP colony numbers after induction of O_{2A}S_{2A}K_{2A}M. FIG. 1M-2 graphically illustrates Oct4-EGFP colony numbers after induction of O_{2A}S_{2A}M. FIG. 1M-3 graphically illustrates Oct4-EGFP colony numbers after induction of O_{2A}K_{2A}M. FIG. 1M-4 graphically illustrates Oct4-EGFP colony numbers after induction of S_{2A}K_{2A}M. FIG. 1N graphically illustrates pluripotent gene marker expression in S_{2A}K_{2A}M iPSCs compared to embryonic stem cell (ESC) expression of the same markers. FIG. 1O shows EGFP-positive colonies generated from reprogramming neural progenitor cells (NPCs) with S_{2A}K_{2A}M (scale bar, 100 μm). FIG. 1P graphically illustrates pluripotent gene marker expression in S_{2A}K_{2A}M iPSCs from the neural progenitor cell (NPC) reprogramming.

FIG. 2A-2S illustrate that secondary S_{2A}K_{2A}M MEFs (2° MEFs) can be efficiently reprogrammed to pluripotency. FIG. 2A shows a schematic illustrating the derivation of S_{2A}K_{2A}M 2° MEFs and NPCs from embryos obtained from tetraploid complementation assays. FIG. 2B is a western blot illustrating Sox2 and Klf4 protein expression at the indicated times after doxycycline induction of polyprotein expression in S_{2A}K_{2A}M secondary (2°) MEFs. FIG. 2C shows cells illustrating activation of Sox2 and Klf4 in 2° MEFs and NPCs (scale bar, 50 μm). FIG. 2D-1 to 2D-4 illustrates morphological changes of MEFs at day 0 and during the first 3 days of reprogramming (scale bar, 100 μm). FIG. 2D-1 shows an image of MEFs at day 0. FIG. 2D-2 shows an image of MEFs at day 1. FIG. 2D-3 shows an image of MEFs at day 2. FIG. 2D-4 shows an image of MEFs at day 3. FIG. 2E-1 to 2E-4 graphically illustrate activation of various mesenchymal epithelial transition factor (MET) genes during the first 4 days of reprogramming. FIG. 2E-1 graphically illustrates Cdh1 activation during the first 4 days of reprogramming. FIG. 2E-2 graphically illustrates EpCAM activation during the first 4 days of reprogramming. FIG. 2E-3 graphically illustrates Krt8 activation during the first 4 days of reprogramming. FIG. 2E-4 graphically illustrates Ocln activation during the first 4 days of reprogramming. FIG. 2F illustrates activation of Oct4-EGFP in 2° MEFs when cultured under normal ESC conditions (DMSO) and AF conditions (AF: media containing A83-01+Forskolin) (scale bar, 100 μm). FIG. 2G illustrates activation of Oct4-EGFP examined by flow cytometry. FIGS. 2H-1 and 2H-2 graphically illustrate activation of Oct4 and Nanog during MEF reprogramming. FIG. 2H-1 graphically illustrate activation of Oct4 during MEF reprogramming. FIG. 2H-2 graphically illustrate activation of Nanog during MEF reprogramming. FIG. 2I graphically illustrates EGFP-positive colony formation efficiency with or without small molecules (A: A83-01; F: Forskolin). Three conditions (A, F, and AF) were compared to control samples (DMSO). FIG. 2J graphically illustrates EGFP-positive colony formation efficiency under different cell densities. FIG. 2K graphically illustrates EGFP-positive colony formation efficiency measured by initial nuclei counting. FIG. 2L graphically illustrates EGFP-positive colony formation efficiency measured by single-cell seeding. FIG. 2M shows cells that were immunofluorescent-stained for Oct4 and Nanog proteins at the end of reprogramming. FIG. 2N graphically illustrates the timing of EGFP-positive colony formation (i.e., iPSC generation) induced by S_{2A}K_{2A}M in 2° MEFs. Data in FIGS. 2E, 2I, and 2J represent mean±SD (n>3). p values were determined by one-way ANOVA with Bonferroni post hoc test. *p<0.05; **p<0.01; ns, not significant. FIG. 2O shows in situ and P1 iPSC colonies obtained by reprogramming 2° MEFs (scale bar, 100 μm). FIG. 2P graphically illustrates expression of pluripotent gene markers in S_{2A}K_{2A}M 2° iPSCs compared to embryonic stem cells (ESCs). FIG. 2Q graphically illustrates colony numbers generated from 2° NPCs with or without AF. (AF: A83-01, Forskolin). FIG. 2R graphically illustrates the efficiency of EGFP-positive colony formation from 2° NPCs as measured by counting cell nuclei numbers before and after adding doxycycline. FIG. 2S graphically illustrates the timing of iPSC generation from 2° NPCs expressing S_{2A}K_{2A}M. Doxycycline induction of polyprotein expression had been removed for the number of days indicated.

FIG. 3A-30 illustrate the importance of Sox2 and Klf4 stoichiometry for S_{2A}K_{2A}M reprogramming. FIG. 3A shows a schematic illustrating three factor combinations S+K_{2A}M, K+S_{2A}M, M+S_{2A}K, and S+K+M, where the plus sign indicates that a single (monocistronie) factor was expressed either with the polycistronic factors or with other single (‘monocistronic’) factors. FIGS. 3B-1 and 3B-2 illustrate Sox2 and Klf4 immunofluorescently stained cells transduced with polycistronic S_{2A}K_{2A}M and monocistronic S+K+M expression vectors (scale bar, 100 μm). Three single cells indicated in the left image were enlarged and highlighted to the right. FIG. 3B-1 shows illustrate Sox2 and Klf4 immunofluorescently stained cells transduced with separate polycistronic S_{2A}K_{2A}M expression vectors (scale bar, 100 μm). FIG. 3B-2 shows illustrate Sox2 and Klf4 immunofluorescently stained cells transduced with monocistronic S+K+M expression vectors (scale bar, 100 μm). FIGS. 3C-1 and 3C-2 shows scatter plots illustrating the Sox2 and Klf4 fluorescent intensities in single cells. The y and x axes represent the intensities for Sox2 and Klf4, respectively, and each dot represents one cell. RFU: relative fluorescence unit. FIG. 3C-1 shows a scatter plots illustrating the Sox2 and Klf4 fluorescent intensities in single cells with polycistronic S_{2A}K_{2A}M expression vectors. FIG. 3C-2 shows a scatter plots illustrating the Sox2 and Klf4 fluorescent intensities in single cells with monocistronic S_{2A}K_{2A}M expression vectors. FIG. 3D graphically illustrates EGFP-positive colony numbers for S_{2A}K_{2A}M, S+K_{2A}M, K+S_{2A}M, M+S_{2A}K, and S+K+M transduced cell types. FIG. 3E is a schematic diagram depicting expression cassettes used for added expression of Sox2 (+Sox2) or Klf4 (+Klf4) within S_{2A}K_{2A}M 2° MEFs in FIG. 3F. FIG. 3F-1 to 3F-3 show scatter plots illustrating the Sox2 and Klf4 signal intensities of single cells for the control, +Sox2, and +Klf4 cell types shown in FIG. 3E. The y and x axes represent the intensities for Sox2 and Klf4, respectively. FIG. 3F-1 shows a scatter plot illustrating the Sox2 and Klf4 signal intensities of single control cells. FIG. 3F-2 shows a scatter plots illustrating the Sox2 and Klf4 signal intensities of single cells with added +Sox2. FIG. 3F-3 shows a scatter plots illustrating the Sox2 and Klf4 signal intensities of single cells with added +Klf4. The equation shown in FIG. 3F-1 is provided to indicate the diagonal distribution of cells. This equation was used to measure cell drifting toward high Sox2 or Klf4 in the +Sox2 and +Klf4 conditions shown in FIG. 3E. The percentages of high Sox2 and Klf4 cells is shown in FIG. 3F-1 to 3F-3. RFU: relative fluorescence unit. FIGS. 3G-1 and 3G-2 graphically illustrate Sox2 and Klf4 expression levels in the cell lines expressing added Sox2 (+Sox2) or added Klf4 (+Klf4) on day 2. FIG. 3G-1 graphically illustrates Sox2 expression levels in the cell lines expressing added Sox2 (+Sox2) on day 2. FIG. 3G-2 graphically illustrates Klf4 expression levels in the cell lines expressing added Klf4 (+Klf4) on day 2. FIG. 3H graphically illustrates endogenous Oct4 activation in the +Sox2 and +Klf4 cells on day 4 when using the expression systems shown in FIG. 3E. FIG. 3I graphically illustrates the number of EGFP-positive colonies per 8000 cells for +Sox2 and +Klf4 cell cultures on day 12 when using the expression systems shown in FIG. 3E. The efficiency is shown for each cell type. FIG. 3J shows schematics depicting the following three polycistronic expression cassettes K_{2A}M, S_{2A}M, S_{2A}K, and the monocistronic expression cassettes S+K. FIG. 3K graphically illustrates the number of EGFP-positive colonies per 100,000 cells for K_{2A}M, S_{2A}M, S_{2A}K, and S+K cell types, as a measure of the efficiency of generating iPSCs. FIG. 3L graphically illustrates expression of pluripotent gene markers within S_{2A}K iPSCs. R1 mouse ESCs were used for control. FIG. 3M shows Oct4-EGFP colonies in situ and at passage 1 that were generated from expression of S_{2A}K (scale bar, 100 μm). Data in FIGS. 3D, 3G, 3H, and 3I represent mean±SD (n>3). p values were determined by one-way ANOVA with Bonferroni post hoc test. **p<0.01. FIG. 3N-1 to 3N-3 illustrate Sox2 and Klf4 signal intensities for single cells with the indicated expression systems. FIG. 3N-1 illustrates Sox2 and Klf4 signal intensities for single S+K_{2A}M cell types. FIG. 3N-2 illustrates Sox2 and Klf4 signal intensities for single K+S_{2A}M cell types. FIG. 3N-3 illustrates Sox2 and Klf4 signal intensities for single M+S_{2A}K cell types. The y and x axes represent the intensities for Sox2 and Klf4, respectively, after 48 hours of doxycycline induction and the dashed lines represent the threshold for positive signals of Sox2 and Klf4 staining. The numerical percentages of cells co-expressing Sox2 and Klf4 are also provided. RFU: relative fluorescence unit. FIG. 3O graphically illustrates the percent of cells that express both Sox2 and Klf4 (co-expression efficiencies) in S_{2A}K_{2A}M, S+K_{2A}M, K+S_{2A}M, M+S_{2A}K, and S+K+M cultures.

FIG. 4A-4I illustrate Identification of the Transcriptional Switches in MEF Reprogramming and Converging Trajectories in MEF and NPC Reprogramming. FIG. 4A shows a schematic illustrating the RNA samples collected for RNA sequencing at different time points. FIG. 4B shows Principal Components Analysis (PCA) for MEF reprogramming depicting the reprogramming progression from MEFs to iPSCs. Data for days 0 (hearts), 2 (stars), 4 (triangles), 8 (pentagons), 12 (diamonds), and iPSC/ESC (circles) samples are shown. Each sample has two replicates except for iPSC and ESC. FIG. 4C illustrates hierarchical clustering analysis for MEF reprogramming intermediates. FIG. 4D illustrates correlation analysis for MEF reprogramming intermediates. For each time point, two replicates were used. FIG. 4E graphically illustrates differential expressed gene (DEG) numbers found between successive intermediates during MEF reprogramming. FIG. 4F graphically illustrates comparison of MEF and NPC reprogramming trajectories. Cells were projected to the first two (dash lines) or three principle components of Principal Components Analysis (PCA). Circles and squares represent MEF and NPC reprogramming intermediates, respectively. Data for samples at days 0 (hearts), 2 (stars), 8 (pentagons), 12 (diamonds), and iPSC/ESC (circles) are shown. Each sample had two replicates except for iPSC and ESC. FIG. 4G graphically illustrates differential expressed gene (DEG) numbers between intermediates of the same time points from MEF and NPC reprogramming. FIG. 4H shows a schematic model for the converging trajectories of MEF and NPC reprogramming over time. FIG. 4I graphically illustrates the number of EGFP-positive colonies from EGFP-positive and EGFP-negative populations during MEF and NPC reprogramming. EGFP-positive and negative populations were sorted on day 6 and replated to continue reprogramming.

FIG. 5A-5G illustrate removal of the MEF identity and activation of pluripotency network during MEF reprogramming. FIG. 5A illustrates the expression profile of genes changed in the day 0/2 transcriptional switch. Upregulated and downregulated genes were further divided into two subgroups based on their further expression changes. The gene numbers are shown in the parentheses. FIG. 5B-1 to 5B-3 show that Thy1, Col6a2, and S100s4 were downregulated on day 2 during MEF reprogramming. FIG. 5B-1 shows that Thy1 was downregulated on day 2 during MEF reprogramming. FIG. 5B-2 shows that Col6a2 was downregulated on day 2 during MEF reprogramming. FIG. 5B-3 shows that S100s4 was downregulated on day 2 during MEF reprogramming. FIG. 5C illustrates expression profiles of genes that were upregulated during MEF reprogramming. The genes were further divided into groups according to their first time of activation by twofold. Activated pluripotent genes were listed on the right according to their activation time shown on the left. FIG. 5D shows a heatmap illustrating the activation kinetics of pluripotent genes during MEF reprogramming. The highest-level during reprogramming was set as 1 (100%) for normalization. FIG. 5E graphically illustrates activation of Oct4, Zfp296, and Lin28a/b as verified by qPCR on different reprogramming days. FIG. 5F illustrates correlation analysis for MEF and NPC reprogramming intermediates with 112 pluripotency-associated genes. Cell populations from the same time points were highlighted with box frames. FIG. 5G shows a schematic model for the converging trajectories of MEF and NPC reprogramming. Original cell identities were removed during the day 0/2 transcriptional switch, and pluripotency network was gradually established afterwards.

FIG. 6A-6M illustrate that Sox2 and Klf4 cooperate to activate pluripotency network in S_{2A}K_{2A}M reprogramming. FIG. 6A illustrates de novo discovery of peak motifs bound by Sox2 and Klf4 in chromosomal immunoprecipitation experiments. FIG. 6B illustrates distance analysis of Sox2 and Klf4 motifs in Sox2 peaks. FIG. 6C shows direct interaction of Sox2 and Klf4 as verified by co-immunoprecipitation in day 2 reprogramming MEFs. FIG. 6D shows a Venn diagram illustrating the overlap of Sox2 and Klf4 peak sites. FIG. 6E shows heatmaps of Sox2, Klf4, and H2K27 acetylation ChIP-seq signals for the indicated groups of peaks, sorted by the intensity of Sox2 in Sox_Klf and Sox_solo, and by the intensity of Klf4 in Klf_solo. FIG. 6F illustrates quantification of signal intensities of Sox2, Klf4, and H3K27 acetylation from the data in FIG. 6E. FIG. 6G-1 to 6G-3 show boxplots showing the expression of genes associated with the Sox_Klf, Sox_solo, and Klf_solo peaks. FIG. 6G-1 shows boxplots illustrating expression of genes associated with the Sox_Klf peaks. FIG. 6G-2 shows boxplots illustrating expression of genes associated with the Sox_solo peaks. FIG. 6G-3 shows boxplots illustrating expression of genes associated with the Klf_solo peaks. FIG. 6H shows a Venn diagram illustrating the binding overlap of Sox2 in S_{2A}K_{2A}M and Sox2_tetO conditions. FIG. 6I illustrates de novo discovery of motifs with Sox2 binding peaks in Sox2_tetO condition. FIG. 6J illustrates quantification of signal intensities of Sox2 and H3K27 acetylation in three different groups of Sox2 binding peaks. Sox2_co indicates the shared peaks in S_{2A}K_{2A}M and Sox2_tetO condition, Sox_SKM indicates the peaks specific for S_{2A}K_{2A}M reprogramming, and Sox tetO indicates peaks specific for Sox2_tetO condition. In the upper right corner, SKM (solid line) represents S_{2A}K_{2A}M reprogramming, and Sox2 (dashed line) represents the Sox2_tetO condition for the top three panels. In the lower right corner, the solid line indicates Day 0 of reprogramming and the dashed line indicates Day 2 of reprogramming for the bottom three panels. FIG. 6K illustrates Sox2 and Klf4 bindings and H3K27 acetylation sites along the Oct4 enhancer of the Oct4 regulatory region of chromosome 17. The locations of super-enhancer and ChIP-qPCR amplicons (a through i) are also shown. FIG. 6L illustrates Sox2 and Klf4 bindings at the Oct4 enhancer as examined by ChIP-qPCR on reprogramming day 2, where a-i are as shown in FIG. 6K. FIG. 6M illustrates Sox2 and Klf4 bindings at the Oct4 enhancer as examined by ChIP-qPCR on reprogramming day 5, where a-i are as shown in FIG. 6K.

### DETAILED DESCRIPTION

As described herein, in the absence of ectopic Oct4 expression, polycistronic Sox2, Klf4, and c-Myc was sufficient to establish pluripotency in several types of differentiated somatic cells. In some cases, c-Myc was not needed. The stoichiometry of Sox2 and Klf4 was important for this reprogramming, as disruption of the factor balance led to a significant decrease or failure in iPSC generation. To optimize the stoichiometry of Sox2 and Klf4, polycistronic expression cassettes are described herein that include a promoter operably linked to a nucleic acid segment encoding Sox2, Klf4, and optionally c-Myc. The nucleic acid segment can also include one or more peptide linkers between the Sox2, Klf4, and optional c-Myc coding regions. For example, the 2A “self-cleaving” peptides can be used as peptide linkers between the Sox2, Klf4, and optional c-Myc coding regions. Such linkers provide cleavage between the Sox2, Klf4, and optional c-Myc polypeptides. One example of a polycistronic expression cassette can, for example, include an open reading frame that includes the Sox2, Klf4, and c-Myc coding regions, where there is a cleavable 2A peptide linker between and in frame with the Sox2 and Klf4 coding regions, and where there is a 2A peptide linker between and in frame with the Klf4 and c-Myc coding regions (referred to as S_{2A}K_{2A}M). Examples of cleavable linker sequences are provided herein.

A “Klf polypeptide” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J. & Yang, V. W. Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. See, Nakagawa, et al., Nature Biotechnology 26:101-106 (2007). In some embodiments, variants have at least 85%, 90%, 95%, 97%, 98%, 99%, or 99.5% amino acid sequence identity across their whole sequence compared to a naturally occurring Klf polypeptide family member such as to those listed above or such as listed in Genbank. Klf polypeptides (e.g., Klf1, Klf4, and Klf5) can be from human, mouse, rat, bovine, porcine, or other animals. Generally, the same species of protein will be used with the species of cells being manipulated.

The Klf4 polypeptide can be used as a pluripotency factor encoded in the polycistronic expression cassette. For example, the Klf4 polypeptide employed can have NCBI accession no. CAX16088 (mouse Klf4), NP_004226.3 (GI: 194248077) (human Klf4), or NP_001300981.1 (GI: 930697457) (human Klf4). A sequence for human Klf4 accession no. NP_004226.3 (GI: 194248077) is shown below as SEQ ID NO:1.

| | |
|---|---|
| 1 | MRQPPGESDM AVSDALLPSE STFASGPAGR EKTLRQAGAP |
| 41 | NNRWREELSH MKRLPPVLPG RPYDLAAATV ATDLESGGAG |
| 81 | AACGGSNLAP LPRRETEEFN DLLDLDFILS NSLTHPPESV |
| 121 | AATVSSSASA SSSSSPSSSG PASAPSTCSF TYPIRAGNDP |
| 161 | GVAPGGTGGG LLYGRESAPP PTAPFNLADI NDVSPSGGFV |
| 201 | AELLRPELDP VYIPPQQPQP PGGGLMGKFV LKASLSAPGS |
| 241 | EYGSPSVISV SKGSPDGSHP VVVAPYNGGP PRTCPKIKQE |
| 281 | AVSSCTHLGA GPPLSNGHRP AAHDFPLGRQ LPSRTTPTLG |
| 321 | LEEVLSSRDC HPALPLPPGF HPHPGPNYPS FLPDQMQPQV |
| 361 | PPLHYQELMP PGSCMPEEPK PKRGRRSWPR KRTATHTCDY |
| 401 | AGGGKTYTKS SHLKAHLRTH TGEKPYHCDW DGCGWKFARS |
| 441 | DELTRHYRKH TGHRPFQCQK CDRAFSRSDH LALHMKRHF |

The SEQ ID NO:1 Klf4 polypeptide is encoded, for example, by a cDNA with NCBI accession number Klf4 NM 004235.6.

The sequence for human Klf4 accession no. NP_001300981.1 (GI: 930697457) is shown below as SEQ ID NO:2.

| | |
|---|---|
| 1 | MRQPPGESDM AVSDALLPSE STFASGPAGR EKTLRQAGAP |
| 41 | NNRWREELSH MKRLPPVLPG RPYDLAAATV ATDLESGGAG |
| 61 | AACGGSNLAP LPRRETEEFN DLLDLDFILS NSLTHPPESV |
| 121 | AATVSSSASA SSSSSPSSSG PASAPSTCSF TYPIRAGNDP |
| 161 | GVAPGGTGGG LLYGRESAPP PTAPFNLADI NDVSPSGGFV |
| 201 | AELLRPELDP VYIPPQQPQP PGGGLMGKFV LKASLSAPGS |
| 241 | EYGSPSVISV SKGSPDGSHP VVVAPYNGGP PRTCPKIKQE |
| 281 | AVSSCTHLGA GPPLSNGHRP AAHDFPLGRQ LPSRTTPTLG |
| 321 | LEEVLSSRDC HPALPLPPGF HPHPGPNYPS FLPDQMQPQV |
| 361 | PPLHYQGQSR GFVARAGEPC VCWPHFGTHG MMLTPPSSPL |
| 401 | ELMPPGSCMP EEPKPKRGRR SWPRKRTATH TCDYAGCGKT |
| 441 | YTKSSHLKAH LRTHTGEKPY HCDWDGCGWK FARSDELTRH |
| 481 | YRKHTGHRPF QCQKCDRAFS RSDHLALHMK RHF |

The SEQ ID NO:2 Klf4 polypeptide is encoded, for example, by a cDNA with NCBI accession number Klf4 NM_001314052.2.

A “Sox polypeptide” refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, e.g., Dang, D. T., et al., Int. J. Biochem Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. See, Nakagawa, et al., Nature Biotechnology 26:101-106 (2007). In some embodiments, variants have at least 85%, 90%, 95%, 97%, 98%, 99%, or 99.5% amino acid sequence identity across their whole sequence compared to a naturally occurring Sox polypeptide family member such as to those listed above or such as listed in Genbank. Sox polypeptides (e.g., Sox1, Sox2, Sox3, Sox15, or Sox18) can be from human, mouse, rat, bovine, porcine, or other animals. Generally, the same species of protein will be used with the species of cells being manipulated. The Sox2 polypeptide can be used as a pluripotency factor encoded in the polycistronic expression cassette.

For example, the Sox2 polypeptide encoded in the polycistronic expression cassette can have accession number CAA83435 (human Sox2), which has the following sequence (SEQ ID NO:3).

| | |
|---|---|
| 1 | HSARMYNMME TELKPPGPQQ TSGGGGGNST AAAAGGNQKN |
| 41 | SPDRVKRPMN AFMVWSRGQR RKMAQENPKM HNSEISKRLG |
| 81 | AEWKLLSETE KRPFIDEAKR LRALHMKEHP DYKYRPRRKT |
| 121 | KTLMKKDKYT LPGGLLAPGG NSMASGVGVG AGLGAGVNQR |
| 161 | MDSYAHMNGW SNGSYSMMQD QLGYPQHPGL NAHGAAQMQP |
| 201 | MHRYDVSALQ YNSMTSSQTY MNGSPTYSMS YSQQGTPGMA |
| 241 | LGSMGSVVKS EASSSPPVVT SSSHSRAPCQ AGDLRDMISM |
| 281 | YLPGAEVPEP AAPSRLHMSQ HYQSGPVPGT AINGTLPLSH |
| 341 | M |

The Sox2 polypeptide is encoded, for example, by a cDNA with NCBI accession number NM_003106.4.

A “Myc polypeptide” refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, 95%, 97%, 98%, 99%, or 99.5% amino acid sequence identity across their whole sequence compared to a naturally occurring Myc polypeptide family member, such as to those listed above or such as listed in Genbank. Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor. For example, in some cases the Myc polypeptide can be a human Myc polypeptide with accession number CAA25015 (human Myc), which has the following sequence (SEQ ID NO:4).

| | |
|---|---|
| 1 | MPLNVSFTNR NYDLDYDSVQ PYFYCDEEEN FYQQQQQSEL |
| 41 | QPPAPSEDIW KKFELLPTPP LSPSRRSGLC SPSYVAVTPF |
| 61 | SLRGDNDGGG GSFSTADQLE MVTELLGGDM VNQSFICDPD |
| 121 | DETFIKNIII QDCMWSGFSA AAKLVSEKLA SYQAARKDSG |
| 161 | SPNPARGHSV CSTSSLYLQD LSAAASECID PSVVFPYPLN |
| 201 | DSSSPKSCAS QDSSAFSPSS DSLLSSTESS PQGSPEPLVL |
| 241 | HEETPPTTSS DSEEEQEDEE EIDVVSVEKR QAPGKRSESG |
| 281 | SPSAGGHSKP PHSPLVLKRC HVSTHQHNYA APPSTRKDYP |
| 321 | AAKRVKLDSV RVLRQISNNR KCTSPRSSDT EENVKRRTHN |
| 361 | VLERQRRNEL KRSFFALRDQ IPELENNEKA PKVVILKKAT |
| 401 | AYILSVQAEE QKLISEEDLL RKRREQLKHK LEQLRNSCA |

The Myc polypeptide with SEQ ID NO:4 is partially encoded, for example, by a nucleic acid with NCBI accession number X00196.1.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. e.g., Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, 95%, 97%, 98%, 99%, or 99.5% amino acid sequence identity across their whole sequence compared to a naturally occurring Oct polypeptide family member such as to those listed above or such as listed in Genbank accession number NP002692.2 (human Oct4) or NP038661.1 (mouse Oct4). Oct polypeptides (e.g., Oct3/4) can be from human, mouse, rat, bovine, porcine, or other animals. Generally, the same species of protein will be used with the species of cells being manipulated. The Oct polypeptide(s) can be a pluripotency factor.

One example of an Oct4 polypeptide sequence is available in the NCBI database with accession number NP002692.2 (human Oct4), shown below as SEQ ID NO:5.

| | |
|---|---|
| 1 | MAGHLASDFA FSPPPGGGGD GPGGPEPGWV DPRTWLSFQG |
| 41 | PPGGPGIGPG VGPGSEVWGI PPCPPPYEFC GGMAYCGPQV |
| 81 | GVGLVPQGGL ETSQPEGEAG VGVESNSDGA SPEPCTVTPG |
| 121 | AVKLEKEKLE QNPEESQDIK ALQKELEQFA KLLKQKRITL |
| 161 | GYTQADVGLT LGVLFGKVFS QTTICRFEAL QLSFKNMCKL |
| 201 | RPLLQKWVEE ADNNENLQEI CKAETLVQAR KRKRTSIENR |
| 241 | VRGNLENLFL QCPKPTLQQI SHIAQQLGLE KDVVRVWFCN |
| 281 | RRQKGKRSSS DYAQREDFEA AGSPFSGGPV SFPLAPGPHF |
| 321 | GTPGYGSPHF TALYSSVPFP EGEAFPPVSV TTLGSPMHSN |

A cDNA nucleotide sequence for the human Oct4 polypeptide having SEQ ID NO:5 is available in the NCBI database as accession number NM_002701.4 (GI:116235483), which is shown below as SEQ ID NO:6.

| | |
|---|---|
| 1 | CCTTCGCAAG CCCTCATTTC ACCAGGCCCC CGGCTTGGGG |
| 41 | CGCCTTCCTT CCCCATGGCG GGACACCTGG CTTCGGATTT |
| 81 | CGCCTTCTCG CCCCCTCCAG GTGGTGGAGG TGATGGGCCA |
| 121 | GGGGGGCCGG AGCCGGGCTG GGTTGATCCT CGGACCTGGC |
| 161 | TAAGCTTCCA AGGCCCTCCT GGAGGGCCAG GAATCGGGCC |
| 201 | GGGGGTTGGG CCAGGCTCTG AGGTGTGGGG GATTCCCCCA |
| 241 | TGCCCCCCGC CGTATGAGTT CTGTGGGGGG ATGGCGTACT |
| 281 | GTGGGCCCCA GGTTGGAGTG GGGCTAGTGC CCCAAGGCGG |
| 321 | CTTGGAGACC TCTCAGCCTG AGGGCGAAGC AGGAGTCGGG |
| 361 | GTGGAGAGCA ACTCCGATGG GGCCTCCCCG GAGCCCTGCA |
| 401 | CCGTCACCCC TGGTGCCGTG AAGCTGGAGA AGGAGAAGCT |
| 441 | GGAGCAAAAC CCGGAGGAGT CCCAGGACAT CAAAGCTCTG |
| 481 | CAGAAAGAAC TCGAGCAATT TGCCAAGCTC CTGAAGCAGA |
| 521 | AGAGGATCAC CCTGGGATAT ACACAGGCCG ATGTGGGGCT |
| 561 | CACCCTGGGG GTTCTATTTG GGAAGGTATT CAGCCAAACG |
| 601 | ACCATCTGCC GCTTTGAGGC TCTGCAGCTT AGCTTCAAGA |
| 641 | ACATGTGTAA GCTGCGGCCC TTGCTGCAGA AGTGGGTGGA |
| 681 | GGAAGCTGAC AACAATGAAA ATCTTCAGGA GATATGCAAA |
| 721 | GCAGAAACCC TCGTGCAGGC CCGAAAGAGA AAGCGAACCA |
| 761 | GTATCGAGAA CCGAGTGAGA GGCAACCTGG AGAATTTGTT |
| 801 | CCTGCAGTGC CCGAAACCCA CACTGCAGCA GATCAGCCAC |
| 841 | ATCGCCCAGC AGCTTGGGCT CGAGAAGGAT GTGGTCCGAG |
| 881 | TGTGGTTCTG TAACCGGCGC CAGAAGGGCA AGCGATCAAG |
| 921 | CAGCGACTAT GCACAACGAG AGGATTTTGA GGCTGCTGGG |
| 961 | TCTCCTTTCT CAGGGGGACC AGTGTCCTTT CCTCTGGCCC |
| 1001 | CAGGGCCCCA TTTTGGTACC CCAGGCTATG GGAGCCCTCA |
| 1041 | CTTCACTGCA CTGTACTCCT CGGTCCCTTT CCCTGAGGGG |
| 1081 | GAAGCCTTTC CCCCTGTCTC CGTCACCACT CTGGGCTCTC |
| 1121 | CCATGCATTC AAACTGAGGT GCCTGCCCTT CTAGGAATGG |
| 1161 | GGGACAGGGG GAGGGGAGGA GCTAGGGAAA GAAAACCTGG |
| 1201 | AGTTTGTGCC AGGGTTTTTG GGATTAAGTT CTTCATTCAC |
| 1241 | TAAGGAAGGA ATTGGGAACA CAAAGGGTGG GGGCAGGGGA |
| 1281 | GTTTGGGGCA ACTGGTTGGA GGGAAGGTGA AGTTCAATGA |
| 1321 | TGCTCTTGAT TTTAATCCCA CATCATGTAT CACTTTTTTC |
| 1361 | TTAAATAAAG AAGCCTGGGA CACAGTAGAT AGACACACTT |
| 1401 | AAAAAAAAAA A |

The nucleic acid segments encoding Sox2, Klf4, and optionally c-Myc, are joined to form a larger polycistronic nucleic acid segment. As illustrated herein, the positions of the Sox2, Klf4, and optional c-Myc coding regions within the polycistronic nucleic acid can vary. In some cases, the Klf4 coding region is 5′ to the Sox2 and optional c-Myc coding regions. In other cases, the Sox2_coding region is 5′ to the Klf4 and optional c-Myc coding regions. In some cases, the cMyc coding region is not included in the polycistronic nucleic acid. In general, the polycistronic nucleic acid is constructed so that the Sox2 and Klf4 polypeptides are expressed at approximately equivalent levels.

Cleavage sites can be included in frame between the segments encoding Sox2, Klf4, and optionally c-Myc. Cleavable peptide linkers to be used between the Klf4, Sox2, and/or c-Myc coding regions can include, for example, 2A or LP4 sequences (de Felipe et al., Trends Biotechnol 24(2):68-75 (2006); Sun et al. Processing and targeting of proteins derived from polyprotein with 2A and LP4/2A as peptide linkers in a maize expression system, PLOS (2017)).

The cleavable linker can have a variety of sequences. The mechanism of 2A-mediated “self-cleavage” involves ribosome skipping the formation of a glycyl-prolyl peptide bond at the C-terminus of the 2A. Hence, the cleavable linker can have a Gly-Pro at its C-terminus linkage junction. A conserved sequence GDVEXNPGP (SEQ ID NO:7) (where X is any amino acid) is shared by different 2A linkers at their C-termini and is needed for generating steric hindrance and ribosome skipping.

The first discovered 2A was F2A (foot-and-mouth disease virus), after which E2A (equine rhinitis A virus), P2A (porcine teschovirus-1 2A), and T2A (thosea asigna virus 2A) were identified. The LP4 linker peptide is from a natural polyprotein occurring in the seed of Impatiens balsamina and can be split between the first and second amino acids during post-translational processing. Examples of cleavable linkers that can be used to link the Sox2 and Klf4, and optionally the c-Myc, proteins together include (where the N-terminal GSG can be present but may not be needed in some cases):

| | P2A linker: |
|---|---|
| (SEQ ID NO: 8) | |
| | GSGATNFSLLKQAGDVEENPGP |
| | T2A linker: |
| (SEQ ID NO: 9) | |
| | GSGEGRGSLLTCGDVEENPGP |
| | E2A linker: |
| (SEQ ID NO: 10) | |
| | GSGQCTNYALLKLAGDVESNPGP |
| | F2A linker: |
| (SEQ ID NO: 11) | |
| | GSGVKQTLNFDLLKLAGDVESNPGP |
| | LP4 linker: |
| (SEQ ID NO: 12) | |
| | SNAADEVAT |
| | LP4/2A linker: |
| (SEQ ID NO: 13) | |
| | SNAADEVATQLLNFDLLKLAGDVESNPGP |
| | 2Am1 linker: |
| (SEQ ID NO: 14) | |
| | APVKQLLNFDLLKLAGDVESNPGP |
| | 2Am2 linker: |
| (SEQ ID NO: 15) | |
| | SGSGQLLNFDLLKLAGDVESNPGP |

An example of an amino acid sequence for a S_{2A}K_{2A}M polypeptide is shown below as SEQ ID NO:16.

| | |
|---|---|
| 1 | MYNMMETELK PPGPQQASGG GGGGGNATAA ATGGNQKNSP |
| 41 | DRVKRPMNAF MVWSRGQRRK MAQENPKMHN SEISKRLGAE |
| 81 | WKLLSETEKR PFIDEAKRLR ALHMKEHPDY KYRPRRKTKT |
| 121 | LMKKDKYTLP GGLLAPGGNS MASGVGVGAG LGAGVNQRMD |
| 161 | SYAHMNGWSN GSYSMMQEQL GYPQHPGLNA HGAAQMQPMH |
| 201 | RYDVSALQYN SMTSSQTYMN GSPTYSMSYS QQGTPGMALG |
| 241 | SMGSVVKSEA SSSPPVVTSS SHSRAPCQAG DLRDMISMYL |
| 281 | PGAEVPEPAA PSRLHMAQHY QSGPVPGTAI NGTLPLSHMA |
| 321 | CGSGEGRGSL LTCGDVEENP GPLEMRQPPG ESDMAVSDAL |
| 361 | LPSFSTFASG PAGREKTLRP AGAPTNRWRE ELSHMKRLPP |
| 401 | LPGRPYDLAA TVATDLESGG AGAACSSNNP ALLARRETEE |
| 441 | FNDLLDLDFI LSNSLTHQES VAATVTTSAS ASSSSSPASS |
| 481 | GPASAPSTCS FSYPIRAGGD PGVAASNTGG GLLYSRESAP |
| 521 | PPTAPFNLAD INDVSPSGGF VAELLRPELD PVYIPPQQPQ |
| 561 | PPGGGLMGKF VLKASLTTPG SEYSSPSVIS VSKGSPDGSH |
| 601 | PVVVAPYSGG PPRMCPKIKQ EAVPSGTVSR SLEAHLSAGP |
| 641 | QLSNGHRPNT HDFPLGRQLP TRTTPTLSPE ELLNSRDCHP |
| 681 | GLPLPPGFHP HPGPNYPPFL PDQMQSQVPS LHYQELMPPG |
| 721 | SCLPEEPKPK RGRRSWPRKR TATHTCDYAG CGKTYTKSSH |
| 761 | LKAHLRTHTG EKPYHCDWDG CGWKFARSDE LTRHYRKHTG |
| 801 | HRPFQCQKCD RAFSRSDHLA LHMKRHFLEG SGQCTNYALL |
| 841 | KLAGDVESNP GPGAPLDFLW ALETPQTATT MPLNVNFTNR |
| 881 | NYDLDYDSVQ PYFICDEEEN FYHQQQQSEL QPPAPSEDIW |
| 921 | KKFELLPTPP LSPSRRSGLC SPSYVAVATS FSPREDDDGG |
| 961 | GGNFSTADQL EMMTELLGGD MVNQSFICDP DDETFIKNII |
| 1001 | IQDCMWSGFS AAAKLVSEKL ASYQAARKDS TSLSPARGHS |
| 1041 | VCSTSSLYLQ DLTAAASEGI DPSVVFPYPL NDSSSPKSCT |
| 1081 | SSDSTAFSPS SDSLLSSESS PRASPEPLVL HEETPPTTSS |
| 1121 | DSEEEQEDEE EIDVVSVEKR QTPAKRSESG SSPSRGHSKP |
| 1161 | PHSPLVLKRC HVSTHQHNYA APPSTRKDYP AAKRAKLDSG |
| 1201 | RVLKQISNNR KCSSPRSSDT EENDKRRTHN VLERQRRNEL |
| 1241 | KRSFFALRDQ IPELENNEKA PKVVILKKAT AYILSIQADE |
| 1281 | HKLTSEKDLL RKRREQLKHK LEQLRNSGA |

An example of an amino acid sequence for a S_{2A}K polypeptide is shown below as SEQ ID NO:17.

| | |
|---|---|
| 1 | MYNMMETELK PPGPQQASGG GGGGGNATAA ATGGNQKNSP |
| 41 | DRVKRPMNAF MVWSRGQRRK MAQENPKMHN SEISKRLGAE |
| 81 | WKLLSETEKR PFIDEAKRLR ALHMKEHPDY KYRPRRKTKT |
| 121 | LMKKDKYTLP GGLLAPGGNS MASGVGVGAG LGAGVNQRMD |
| 161 | SYAHMNGWSN GSYSMMQEQL GYPQHPGLNA HGAAQMQPMH |
| 201 | RYDVSALQYN SMTSSQTYMN GSPTYSMSYS QQGTPGMALG |
| 241 | SMGSVVKSEA SSSPPVVTSS SHSRAPCQAG DLRDMISMYL |
| 281 | PGAEVPEPAA PSRLHMAQHY QSGPVPGTAI NGTLPLSHMA |
| 321 | CGSGEGRGSL LTCGDVEENP GPLEMRQPPG ESDMAVSDAL |
| 361 | LPSFSTFASG PAGREKTLRP AGAPTNRWRE ELSHMKRLPP |
| 401 | LPGRPYDLAA TVATDLESGG AGAACSSNNP ALLARRETEE |
| 441 | FNDLLDLDFI LSNSLTHQES VAATVTTSAS ASSSSSPASS |
| 481 | GPASAPSTCS FSYPIRAGGD PGVAASNTGG GLLYSRESAP |
| 521 | PPTAPFNLAD INDVSPSGGF VAELLRPELD PVYIPPQQPQ |
| 561 | PPGGGLMGKF VLKASLTTPG SEYSSPSVIS VSKGSPDGSH |
| 601 | PVVVAPYSGG PPRMCPKIKQ EAVPSGTVSR SLEAHLSAGP |
| 641 | QLSNGHRPNT HDFPLGRQLP TRTTPTLSPE ELLNSRDCHP |
| 681 | GLPLPPGFHP HPGPNYPPFL PDQMQSQVPS LHYQELMPPG |
| 721 | SCLPEEPKPK RGRRSWPRKR TATHTCDYAG CGKTYTKSSH |
| 761 | LKAHLRTHTG EKPYHCDWDG CGWKFARSDE LTRHYRKHTG |
| 801 | HRPFQCQKCD RAFSRSDHLA LHMKRHF |

### Cell Transformation

Polycistronic nucleic acid segments encoding Sox2, Klf4, and optionally c-Myc, can be introduced into cells to facilitate conversion of cells into stem cells (e.g., pluripotent stem cells), or into other cell types. Nucleic acid segments encoding Sox2, Klf4, and optionally c-Myc can be inserted into or employed with any suitable expression system. The polycistronic Sox2, Klf4, and optionally c-Myc nucleic acids can be part of an expression cassette or expression vector that includes a promoter segment operably linked to the nucleic acid segment encoding the Sox2, Klf4, and optionally c-Myc.

Recombinant expression is usefully accomplished using a vector. Vectors include but are not limited to plasmids, viral nucleic acids, viruses, phage nucleic acids, phages, cosmids, and artificial chromosomes. The vector can also include other elements required for transcription (and translation if a marker gene or other protein encoded segment is included in the vector). Such expression cassettes and/or expression vectors can express sufficient amounts of the Sox2, Klf4, and optionally c-Myc to increase conversion of starting cells into stem cells or into cells of another phenotypic lineage.

Expression vectors and/or expression cassettes encoding polycistronic Sox2, Klf4, and optionally c-Myc can include promoters for driving the expression (transcription) of the polycistronic Sox2, Klf4, and optionally c-Myc. The vector can include a promoter operably linked to a polycistronic nucleic acid segment encoding Sox2, Klf4, and optionally c-Myc. Expression can include transcriptional activation, where transcription is increased above basal levels in the target starting cell by 10-fold or more, by 100-fold or more, such as by 1000-fold or more.

As used herein, vector refers to any carrier containing exogenous DNA. Thus, vectors are agents that transport the exogenous nucleic acid into a cell without degradation and include a promoter yielding expression of the polycistronic Sox2, Klf4, and optionally c-Myc in the cells into which it is delivered. A variety of prokaryotic and eukaryotic expression vectors are suitable for carrying, encoding and/or expressing polycistronic Sox2, Klf4, and optionally c-Myc mRNA. Such expression vectors include, for example, TetO-fuw, pET, pET3d, pCR2.1, pBAD, pUC, viral, and yeast vectors. The vectors can be used, for example, in a variety of in vivo and in vitro situations. For example, some of the experimental work illustrated herein involves use of or modification of the TetO-FUW vector.

The expression cassette, expression vector, and sequences in the cassette or vector can be heterologous. The promoter and/or other regulatory segments can be heterologous to the polycistronic segment encoding the Sox2, Klf4, and optionally c-Myc.

As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid segment of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.).

Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In MICROBIOLOGY-1985, AMERICAN SOCIETY FOR MICROBIOLOGY, pp. 229-232, Washington, (1985). For example, such retroviral vectors can include Murine Maloney Leukemia virus (MMLV), and other retroviruses that express desirable properties. Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements.

A “promoter” is generally a sequence or sequences of DNA that function when in a relatively fixed location in regard to the transcription start site. For example, the promoter can be upstream of the coding region for the Sox2, Klf4 and (optionally) c-Myc. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bases in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (e.g., animal, human or nucleated cells) can also contain sequences necessary for the termination of transcription which can affect mRNA expression. For mRNA, these regions are transcribed as polyadenylated segments in the untranslated portion of the mRNA encoding tissue factor protein. The 3′ untranslated regions also include transcription termination sites. The identification and use of 3′ untranslated regions including polyadenylation signals in expression constructs is well established.

The expression of Sox2, Klf4, and optionally c-Myc from a polycistronic expression cassette or expression vector can be controlled by any promoter capable of expression in prokaryotic cells or eukaryotic cells. Such promoters can include ubiquitously acting promoters, inducible promoters, or developmentally regulated promoters. Ubiquitously acting promoters include, for example, a CMV-β-actin promoter. Inducible promoters can include those that are active in particular cell populations or that respond to the presence of drugs such as tetracycline or doxycycline. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include a nucleic acid sequence encoding a marker product. This marker product is used to determine if the gene has been delivered to the cell and once delivered is being expressed. Preferred marker genes are fluorescent proteins, such as red fluorescent protein, green fluorescent protein, yellow fluorescent protein. The E. coli lacZ gene can also be employed as a marker. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1:327 (1982)), mycophenolic acid, (Mulligan, R. C. and Berg, P. Science 209: 1422 (1980)) or hygromycin (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the polycistronic Sox2, Klf4, and optionally c-Myc nucleic acid segment, expression cassette and/or vector can be introduced to a cell by any method including, but not limited to, calcium-mediated transformation, electroporation, microinjection, lipofection, particle bombardment and the like. The cells can be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

The polycistronic expression cassette(s) and/or expression vector(s) encoding the Sox2, Klf4, and optionally c-Myc can be introduced into starting cells or any cell subjected to the methods described herein. For example, the cells can be contacted with viral particles that include the expression cassettes. For example, retroviruses and/or lentiviruses are suitable for expression of Sox2, Klf4, and optionally c-Myc. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid of interest are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types and are generated by using ecotropic packaging cell lines such as BOSC23 (Pear et al. (1993) Proc. Natl. Acad. Sci. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) Proc. Natl. Acad. Sci. 85:6460-6464). Retroviruses packaged with xenotropic envelope protein, e.g. AKR env, are capable of infecting most mammalian cell types, except murine cells. The appropriate packaging cell line may be used to ensure that the subject cells are targeted by the packaged viral particles. Suitable methods of introducing the retroviral vectors comprising expression cassettes into packaging cell lines and of collecting the viral particles that are generated by the packaging lines are well known in the art.

The polycistronic expression cassette(s) and/or expression vector(s) encoding the Sox2, Klf4, and optionally c-Myc can be can be integrated into the genomes of the cells, or the polycistronic expression vectors can be maintained episomally for the time needed to redirect the cells to a stem cell lineage. Episomal introduction and expression of pluripotency factors is desirable because the mammalian cell genome is not altered by insertion of the episomal vectors and because the episomal vectors are lost over time. Hence, use of episomal expression vectors allows expression of pluripotency factors for the short time that is needed to convert non-pluripotent mammalian cells to pluripotent cells, while avoiding possible chromosomal mutation and later expression of pluripotency factors during if differentiation into another cell type is desired.

Episomal plasmid vectors with the polycistronic expression cassette(s) encoding the Sox2, Klf4, and optionally c-Myc, can be introduced into mammalian cells as described for example, in Yu et al., Human induced pluripotent stem cells free of vector and transgene sequences, Science 324(5928): 797-801 (2009); United States Patent Application 20120076762, and Okita et al., A more efficient method to generate integration-free human iPS cells, NATURE METHODS 8: 409-412 (2011), the contents of which are specifically incorporated herein by reference in their entireties.

For example, the polycistronic expression cassette can be included within and the Sox2, Klf4, and optionally c-Myc, can be expressed from an episomal vector that has EBNA-1 (Epstein-Barr nuclear antigen-1) and oriP, or Large T and SV40ori sequences so that the vectors can be episomally present and replicated without incorporation into a chromosome.

The polycistronic expression cassettes and/or vectors can be introduced into mammalian cells in the form of DNA, protein or mature mRNA by a technique such as lipofection, binding with a cell membrane-permeable peptide, liposomal transfer/fusion, or microinjection. When in the form of DNA, a vector such as a virus, a plasmid, or an artificial chromosome can be employed. Examples of viral vectors include retrovirus vectors, lentivirus vectors (e.g., according to Takahashi, K. and Yamanaka, S., Cell, 126: 663-676 (2006); Takahashi, K. et al., Cell, 131: 861-872 (2007); Yu, J. et al., Science, 318: 1917-1920 (2007)), adenovirus vectors (e.g., Okita K, et al., Science 322: 949 (2008)), adeno-associated virus vectors, and Sendai virus vectors (Proc Jpn Acad Ser B Phys Biol Sci. 85: 348-62, 2009), the contents of each of which references are incorporated herein by reference in their entireties. Also, examples of artificial chromosome vectors that can be used include human artificial chromosome (HAC), yeast artificial chromosome (YAC), and bacterial artificial chromosome (BAC and PAC) vectors. As a plasmid, a plasmid for mammalian cells can be used (e.g., Okita K, et al., Science 322: 949 (2008)). A vector can contain regulatory sequences such as a promoter, an enhancer, a ribosome binding sequence, a terminator, and a polyadenylation site, so that a pluripotency factor can be expressed.

### Starting Cells

Starting cells are cells targeted for transformation by the polycistronic Sox2, Klf4, and optionally c-Myc expression cassette or expression vector.

A starting population of cells may be derived from essentially any source and may be heterogeneous or homogeneous. The term “selected cell” or “selected cells” is also used to refer to starting cells. In certain embodiments, the cells to be transformed as described herein are adult cells, including essentially any accessible adult cell type(s). The cells can, for example, be autologous or allogeneic cells (relative to a subject to be treated or who may receive the cells). In some cases, the starting cells are adult progenitor cells or adult somatic cells. In still other embodiments, the starting cells include any type of cell from a newborn, including, but not limited to newborn cord blood, progenitor cells, and tissue-derived cells (e.g., somatic cells). In some embodiments, the starting population of cells does not include pluripotent stem cells. In other embodiments, the starting population of cells can include pluripotent stem cells. Accordingly, a starting population of cells that is transformed by the polycistronic Sox2, Klf4, and optionally c-Myc expression cassettes or expression vectors described herein, can be essentially any live cell type, particularly a somatic cell type.

As illustrated herein, fibroblasts can be reprogrammed to cross lineage boundaries and to be directly converted to pluripotent stem cells. However, the polycistronic expression cassettes and vectors can be used to convert or initiate conversion of starting cells to another cell type. Various cell types from all three germ layers have been shown to be suitable for somatic cell reprogramming by genetic manipulation, including, but not limited, to liver and stomach (Aoi et al., Science 321(5889):699-702 (2008); pancreatic f3 cells (Stadtfeld et al., Cell Stem Cell 2: 230-40 (2008); mature B lymphocytes (Hanna et al., Cell 133: 250-264 (2008); human dermal fibroblasts (Takahashi et al., Cell 131, 861-72 (2007); Yu et al., Science 318(5854) (2007); Lowry et al., Proc Natl Acad Sci USA 105, 2883-2888 (2008); Aasen et al., Nat Biotechnol 26(11): 1276-84 (2008); meningiocytes (Qin et al., J Biol Chem 283(48):33730-5 (2008); neural stem cells (DiSteffano et al., Stem Cells Devel. 18(5): (2009); and neural progenitor cells (Eminli et al., Stem Cells 26(10): 2467-74 (2008). Any starting cells can be transformed with the polycistronic Sox2, Klf4, and optionally c-Myc expression cassette or expression vectors described herein to initiate reprogramming to other cell types.

In some embodiments the starting cells can transiently or continuously express Sox2, Klf4, and optionally c-Myc by incubation under cell culture conditions.

### Reprogramming Methods

Starting cells are treated for a time and under conditions sufficient to convert the starting cells across lineage and/or differentiation boundaries to form stem cells, especially pluripotent stem cells, or de-differentiated stem cells that may not be completely pluripotent. This process is referred to as ‘reprogramming.’ In some cases, the pluripotent stem cells or de-differentiated cells so formed can be differentiated into other types of cells (e.g., neural, cardiac, pancreatic, liver and other types of cells, or progenitors of such cells).

The time for conversion of starting cells into induced pluripotent stem cells or de-differentiated stem cells that may not be completely pluripotent can vary. For example, the starting cells can be incubated until stem cell markers are expressed. Such stem cell markers can include Nanog, SSEA1, Oct4, and combinations thereof. In another example, the starting cells can be incubated until markers of a different cell type are expressed. In some cases, the starting cells are incubated for a time sufficient to form teratomas that contain all three germ layers, or that can generate chimeric mice.

The time for conversion of starting cells into induced pluripotent stem cells can therefore vary. For example, the starting cells can be incubated under cell culture conditions for at least about 3 days, or for at least about 4 days, or for at least about 5 days, or for at least about 6 days, or for at least about 7 days, or for at least about 8 days, or for at least about 9 days, or for at least about 10 days, or for at least about 11 days, or for at least about 12 days, or for at least about 13 days, or for at least about 14 days, or for at least about 15 days, or for at least about 16 days, or for at least about 17 days, or for at least about 18 days, or for at least about 19 days.

In some embodiments, the stem cells so formed can be expanded or further incubated under cell culture conditions for about 5 days to about 35 days, or about 7 days to about 33 days, or about 10 days to about 30 days, or about 12 days to about 27 days, or about 15 days to about 25 days, or about 18 days to about 23 days.

The Examples illustrate some of the experiments performed and results obtained during development of the invention.

### Example 1: Materials and Methods

This Example illustrates some of the materials and methods used in the development of the invention.

### Cell Culture

HEK293T/17 cells (female) were cultured in DMEM (Invitrogen) supplemented with 10% FBS.

Mouse embryonic fibroblasts (MEFs) (mixed sex, for male and female embryos were combined to generate the primary cells) were prepared from the E13.5 embryos, and mouse tail tip fibroblasts (TTFs) (male) were derived from a 14-month old adult male mouse. MEFs and TTFs were cultured in MEF medium (DMEM supplemented with 10% FBS and non-essential amino acid (NEAA, Invitrogen)).

Mouse primary neural progenitor cells (NPCs) (mixed sex, for male and female embryos were combined to generated the primary cells) were prepared from the head of E13.5 embryos and maintained on matrigel (BD, 356231)-coated plates in the NPC medium (Neuralbasal medium (Invitrogen), 2% B27 (Invitrogen), 1% GlutaMAX™ (Invitrogen), 1% penicillin/streptomycin (Invitrogen), 2 μg/ml heparin (Sigma Aldrich), 20 ng/ml bFGF (Thermo fisher Scientific), and 20 ng/ml EGF (R&D)).

Mouse ESCs (male) and iPSCs (male) were maintained on feeders in ESC medium (Knock Out-DMEM (Invitrogen) with 5% ES-FBS (Invitrogen) and 15% Knock Out-serum replacement (KSR, Invitrogen), 1% GlutaMAX™, 1% NEAA, 0.1 mM 2-mercaptoethanol (Sigma Aldrich), 10 ng/ml leukemia inhibitory factor (LIF, Millipore), 3 μM CHIR99021 (Selleck), and 1 μM PD0325901 (Selleck)).

For microinjection, iPSCs (male) were maintained under feeder-free N2B27 condition (50% DMEM/F12 (Invitrogen), 50% Neurobasal Medium, 0.5% N2 (Invitrogen), 1% B27, 0.1 mM 2-mercaptoethanol, 10 ng/ml LIF, 25 μg/ml BSA (Invitrogen), 3 μM CHIR99021, and 1 μM PD0325901).

### Mice

OG2 Mice (B6; CBA-Tg(Pou5f1-EGFP)2Mnn/J) (male and female) were from the Jackson Laboratory (004654). CD-1 (ICR) mice (male and female) were from Charles River (#022). OG2 mice were crossed to obtain OG2 MEFs as well as NPCs in the resulting embryos at embryonic day 13.5. Male OG2 mice at 14-months were used for the derivation of TTFs.

Super-ovulated female CD1 (ICR) mice were mated to CD1 (ICR) males for blastocyst preparation and further microinjection experiments. E13.5 embryos of tetraploid complementation assay were used for derivation of secondary MEFs and NPCs.

All animal procedures were approved by the Institutional Animal Care and Use Committee at the Tsinghua University, Beijing; as well as the Institutional Animal Care and Use Committee at the Institute of Zoology, Chinese Academy of Science, Beijing.

### Plasmid Construction

Plasmids generated in this study are listed in Table 1.

TABLE 1
Plasmids generated. Related to STAR Methods.

| | Insert | | Ligation |
|---|---|---|---|
| Plasmid | Fragment | Primer sequence | Method |
| TetO- | OS | F: gaccgatccagcctccgcg | Gibson |
| FUW- | | gccccgGCCATGGCTGGACACC | Assembly |
| OSM | | TG | |
| | | (SEQ ID NO: 18) | |
| | | R: cgttgaggggcatCTCGAG | |
| | | TGGGCCGGGATTTTC | |
| | | (SEQ ID NO: 19) | |
| | M | F: cggcccactcgagATGCCC | |
| | | CTCAACGTGAACTTCAC | |
| | | (SEQ ID NO: 20) | |
| | | R: ttgattatcgataagcttg | |
| | | atatcgGGCGCGCCTTATGCAC | |
| | | (SEQ ID NO: 21) | |
| TetO- | OKM | F: GCA GCTAGC TGCATGGC | T4 |
| FUW- | | TGGACAC (NheI) | ligation |
| OKM | | (SEQ ID NO: 22) | |
| | | R: GCA GAATTC GGCGCGCC | |
| | | TTATGCA (EcoRI) | |
| | | (SEQ ID NO: 23) | |
| TetO- | SKM | F: GCAGAATTCTGCATGTATA | T4 |
| FUW- | | ACATG (EcoRI) | ligation |
| SKM | | (SEQ ID NO: 24) | |
| | | R: GCAGAATTCGGCGCGCCTT | |
| | | ATGCA (EcoRI) | |
| | | (SEQ ID NO: 25) | |
| TetO- | SK | F: GCAGAATTCTGCATGTATA | T4 |
| FUW-SK | | ACATG (EcoRI) | ligation |
| | | (SEQ ID NO: 26) | |
| | | R: GCAGAATTCTTAAAAGTGC | |
| | | CTCTT (EcoRI) | |
| | | (SEQ ID NO: 27) | |
| TetO- | SM | sub-cloned from FUW-SM | T4 |
| FUW- | | (EcoRI) | ligation |
| SM | | | |
| TetO- | KM | sub-cloned from FUW-KM | T4 |
| FUW- | | (EcoRI) | ligation |
| KM | | | |
| TetO- | KM | F: gcctccgcggccccgAATT | Gibson |
| FUW- | | CGCCATGAGGCAGC | Assembly |
| KMS | | (SEQ ID NO: 28) | |
| | | R: ccgctagcTGCACCAGAGT | |
| | | TTCGAAG | |
| | | (SEQ ID NO: 29) | |
| | S | F: ctggtgcaGCTAGCGGCAG | |
| | | CGGCGCC | |
| | | (SEQ ID NO: 30) | |
| | | R: cgataagcttgatatcgAA | |
| | | TTCGGCGCGCCTCACATGTGCG | |
| | | ACAGGGGC | |
| | | (SEQ ID NO: 31) | |
| TetO- | M | F: gcctccgcggccccgAATT | Gibson |
| FUW- | | CGCCATGCCCCTCA | |
| MSK | | (SEQ ID NO: 32) | |
| | | R: ccgctagcTGCACCAGAGT | |
| | | TTCGAAGC | |
| | | (SEQ ID NO: 33) | |
| | SK | F: ctggtgcaGCTAGCGGCAG | Assembly |
| | | CGGCGCC | |
| | | (SEQ ID NO: 34) | |
| | | R: cgataagcttgatatcgAA | |
| | | TTCGGCGCGCCTTAAAAGTGCC | |
| | | TC | |
| | | (SEQ ID NO: 35) | |

TetO-FUW-OSKM (Catalog no. 20321), TetO-FUW-Oct4 (Catalog no. 20323), TetO-FUW-Sox2 (Catalog no. 20326), TetO-FUW-K1f4 (Catalog no. 20322), TetO-FUW-c-Myc (20324), and FUW-M2rtTA (Catalog no. 20342) are from Addgene. See also, Brambrink et al. Cell Stem Cell 2: 151-159 (Feb. 2008). All plasmids in this study are based on the TetO-FUW backbone. For cloning, the backbone was digested with appropriate enzymes and each insert (e.g., the Sox2, Klf4, and c-Myc coding regions) was recovered by gel extraction. All inserts were amplified by PCR using the KOD Xtreme HS Polymerase (Novagen, 71975-3), and ligated into polycistronic expression cassette using T4 ligase or Gibson Assembly Master Mix (NEB, E2611). All plasmids were confirmed by enzyme digestion and sequencing.

### Virus Preparation and Transduction

For lentivirus preparation, HEK293T cells were plated 1 day ahead to reach about 70% confluency for transfection, and VSV-G envelope expressing plasmid pMD2.G (Addgene, 12259) and psPAX2 (Addgene, 12260) were used for lentiviral packaging. Plasmids (1.8 μg) with the gene of interests were mixed with psPAX2 (1.35 μg) and pMD2.G (0.45 μg) for each well of six-well plates, and Lipofectamine® 3000 Reagent (Thermo Fisher Scientific, L3000) was used for transfection. Five to eight hours later, the medium was changed to fresh MEF medium. Supernatant containing the virus was harvested at 48 hours, passed through a 0.45-μM filter to remove the cell debris, and mixed with 1 volume of fresh medium for immediate use.

For infection, mouse embryonic fibroblasts (MEFs) or neural progenitor cells (NPCs) were incubated with the lentiviral supernatant in the presence of 5 μg/ml polybrene (Millipore) for 8 hours or overnight. Medium was changed back to MEF or NPC medium after the infection for cells to recovery.

### Derivation of Mouse Embryonic Fibroblasts

E13.5 embryos were used for MEFs derivation. After the embryo recovery, the head, limbs, and internal organs, especially the gonads, were removed under dissection microscope. The remaining bodies of the embryos were finely minced with two blades and digested in 0.05% Trypsin-EDTA for 15 minutes. MEF medium was then added to stop the trypsinization. Further dissociation of the tissues was performed by pipetting up and down for a few times. Cells were then collected by centrifugation and plated onto 15 cm dishes for expansion (passage 0, P0). MEFs were used before passages 4 for all tests.

### Derivation of Mouse Neural Progenitor Cells

One day prior to the experiment, Poly-D-lysine (PDL)/Laminin coated plates were prepared for NPC cultures. Briefly, 12-well culture plates were filled with PDL (10 μg/ml in distilled water) and incubated overnight at 37° C. incubator. On the next day, the solution was removed from plate wells. The wells were then washed with distilled water for three times and air-dried. Laminin (5 μg/ml in distilled water) was then added and incubated at 37° C. incubator for 4 hours to overnight. Laminin was removed from well before using the plate.

E13.5 embryos were used for NPC derivation. The embryo was decapitated with dissecting forceps. Skin and skull were peeling back from head to expose the brain. The whole brain was picked out using curved forceps and placed into cold DPBS. After rinsing with DPBS twice, brain was placed in a 35-cm dish, finely minced with sharp scissors. The minced tissue was transferred to a 15 ml centrifuge tube and digested with 1 ml of 0.05% Trypsin-EDTA at 37° C. for 7 minutes. To stop the enzymatic reaction, 5 ml of NPC medium was added to tube, followed by centrifugation and removing the supernatant. Tissue pellet was further dissociated with 1 ml of NPC medium by pipetting up and down several times and filtered with a 70 μm cell strainer. Cells were then placed to PDL/Laminin-coated 12-well plate and cultured in NPC medium for several days. During culture, NPCs proliferated and detached from plate to form floating neural spheres (P0). Spheres were then collected and digested to single NPCs with StemPro Accutase (Thermo Fisher Scientific). Since then, NPCs were cultured adherently on matrigel-coated plate for the following passages. NPCs were used before passages 4 for all tests.

### Derivation of Mouse Tail Tip Fibroblasts

For tail tip fibroblast (TTF) derivation, 14-month old adults were used. The tail was peeled, minced into 1 mm pieces, and cultured in a 60-cm dish. Medium was half changed every 3 days until fibroblasts migrated out of the graft pieces. Cells were then passaged and ready for use (P1).

Reprogramming and Derivation of iPSC Lines

Oct4-GFP (OG2) MEFs or TTFs were seeded onto gelatin-coated plates at the density of 10,000 cells/cm². After transduction, cells were allowed to recover in MEF medium for 24-36 hours. Cells were then replated with the density of 10,000 cells/cm², except elsewhere indicated. For NPCs, 5,000 cells/cm² were seeded on Poly-D-lysine (PDL)/Laminin-coated six-well plates. After transduction, cells were allowed to recover in NPC medium for 24-36 hours. To start reprogramming, cultures were switched to reprogramming medium (ESC medium without Chirr99021 and PD0325901) with 1 μg/ml doxycycline. Doxycycline was used to induce expression of protein(s) from the polycistronic expression cassette. Introduction of doxycycline was denoted as day 0. During the entire process, medium was refreshed every other day for the first 10 days and everyday afterwards. From day 10, ESC medium with 1 μg/ml doxycycline was used. EGFP-positive colonies were usually counted on day 12 and ready for iPSC derivation on day 16.

For iPSC line derivation, the reprogramming cultures were incubated with 1 mg/ml collagenase B (Roche) for 20 minutes at 37° C. Single colonies were picked up under microscope and digested in 0.05% trypsin for 5-10 minutes for single-cell suspensions. Cells were then seeded on feeders in normal ESC medium, and these cells are considered as passage 0 (P0) iPSCs.

### Evaluation of EGFP-Positive Colony Efficiency

To calculate the EGFP-positive colony efficiency precisely, 2° MEFs or NPCs were seeded into 48-well plates. 24 hours later (day 0), half of the wells were stained with Heochest 33342 (Thermo Fisher Scientific), and the exact cell numbers in the well were recorded by counting the stained nuclei. The other half of cells was switched to reprogramming medium with 1 μg/ml doxycycline for further culture. During the experiment, medium was changed every other day, and the EGFP-positive colonies were counted on day 12. The final efficiency was calculated by dividing the EGFP-positive colony numbers by the initial cell numbers recorded on day 0.

Another method was also used. Single cells were seeded into the wells of 96-well plates with feeders. The next day, MEF medium was switched to reprogramming medium with 1 μg/ml doxycycline (day 0). During the reprogramming process, medium was changed every 4 days, and EGFP-positive colony numbers were counted on day 16. The efficiency was calculated by dividing the total EGFP-positive colony numbers by the well numbers.

### Blastocyst Microinjection

iPSCs were cultured under N2B27 condition without feeders. On the day of injection, cells were suspended in Blastocyst Injection Medium (25 mM HEPES-buffered DMEM plus 10% FBS, pH 7.4).

For generation of chimeric mice, super-ovulated female CD1 (ICR) mice (4-week old) were mated to CD1 (ICR) males. Morulae (2.5 d post-coitum) were collected and cultured overnight in KSOM medium (Millipore) at 37° C. in 5% CO₂. The next morning, the blastocysts were ready for iPSCs injection, and approximately 10 cells were injected for each blastocyst. Injected blastocysts were cultured in KSOM medium at 37° C. in 5% CO₂ for 1-2 hours and then implanted into uteri of 2.5 d post-coitum pseudo-pregnant CD1 (ICR) female mice.

For tetraploid complementation assay, two cell-stage CD1 (ICR) embryos were electrofused to produce tetraploid embryos, and approximately 10 iPSCs were injected into the reconstructed tetraploid blastocysts. Injected blastocysts were cultured in KSOM medium at 37° C. in 5% CO₂ for 1-2 hours and then implanted into uteri of 0.5 d post-coitum pseudo-pregnant CD1 (ICR) female mice. E13.5 embryos were dissected for generation of secondary MEFs and NPCs (2° MEFs and NPCs).

For gonadal contribution, the injected embryos were recovered 13 days (E13.5) after implantation. The gonadal regions of each embryo were collected and visualized under microscope for EGFP signal.

### Examination of Secondary Reprogramming System

To validate the induction of reprogramming factors, 2° MEFs and NPCs were plated on 24-well plate at the density of 20,000 cells/cm². After cultured in reprogramming medium with 1 μg/ml doxycycline for 48 hours, cells were fixed for immunofluorescent staining to test the expression of Sox2 and Klf4.

To test the influence of original cell density to final reprogramming efficiency, 2° MEFs and NPCs were plated on feeders in 12-well plates at density of 500 cells/well, 1,000 cells/well, 2.000 cells/well, 4,000 cells/well, respectively. Cells were reprogrammed as previously described. On reprogramming day 12, EGFP positive colony numbers were counted under fluorescent microscope.

To validate the requirement of doxycycline during reprogramming, 2° MEFs and NPCs were plated on feeders in 12-well plates at the density of 1,000 cells/well. Doxycycline was removed from reprogramming medium from day 0 to day 12. EGFP-positive colony numbers were counted on day 16.

To test the reprogramming kinetics with small molecules, 2° MEFs and NPCs were plated on feeders in 12-well plates at density of 1,000 cells/well. Cells were cultured in reprogramming medium with 1 μg/ml doxycycline, 1 μM A83-01 and 10 Forskolin for 12 days. The cell morphology was recorded for the reprogramming kinetics. All conditions were repeated in triplicate.

### Reprogramming of Early EGFP-Positive Cells

Secondary (2°) MEFs and NPCs were seeded on feeders and reprogrammed as described before. On the reprogramming day 6, EGFP-positive and EGFP-negative cells were sorted by flow cytometry, and cells of the same number were replated to a new 6-well plate with feeders, respectively. Cells were cultured in reprogramming medium with 1 μg/ml doxycycline for another 6 more days and the number of EGFP-positive colony was counted.

### Teratoma Formation

To generate teratoma, iPSCs maintained on feeders were switched to matrigel-coated plate and cultured in ESC medium without Chirr99021 and PD0325901. Then iPSCs were trypsinized and suspended in culture medium containing 2% matrigel. Then 1.0×10⁶ cells were subcutaneously injected into the hind limbs of SCID mice. 5 weeks after the injection, tumors were dissected and fixed in 4% of polyformaldehyde (Sigma Aldrich), followed by paraffin section and haematoxylin-eosin (HE) staining.

### Bisulfite Sequencing

Bisulfite treatment was done with the EpiTect Bisulfite Kit (Qiagen, 59104) exactly following the protocol provided for cultured cells. Recovered DNA was amplified by two-round PCR with primers targeting the Oct4 promoter, and the PCR products were ligated with T-vectors pMD20 (Clontech, 3270). Ten random selected clones were sequenced. PCR primers used are listed in Table 2.

### Karyotyping

Karyotype analysis of iPS cell lines was performed at Cell Line Genetics by analyzing the Giemsa binding (Meisner and Johnson, 2008). Briefly, iPSCs undergoing active division were blocked at metaphase with 0.1 μg/ml colcemid. Then iPSCs were trypsinized to single cells by 0.05% trypsin-EDTA. KCL hypotonic solution (0.075M) was used to resuspend and swollen iPSCs by gently swirling and incubating at room temperature for 20 min. Subsequently, iPSCs were fixed in fixative (3:1 v/v ratio of methanol to acetic acid), followed by preparation of slides for karyotyping.

### Flow Cytometry

Reprogramming cells were treated with 1 mg/ml of Collagenase B for 10-30 min depending on the cell density, followed by 5-minute trypsinization with 0.05% trypsin. Cells were then suspended in culture medium and filtered through 40 μm cell strainer. Flow cytometry analysis or sorting was performed on BD FACS Aria III. The treatments with collagenase B, filtration, and sorting usually lead to decrease by 30-50 times in generating EGFP-positive colonies. All data were analyzed with FlowJo v10.

### Western Blots and Quantification

Cell lysis samples or immunoprecipitation (IP) samples were loaded onto 10% SDS-PAGE gel for separation and then transferred to nitrocellulose membranes 0.45 μm (BioRad, 1620115). The following antibodies were used for immuno-blotting (IB): anti-Oct4 (Abcam ab19857), anti-Sox2 (Millipore AB5603 for IP, Abcam ab79351 for IB), anti-Klf4 (Stemgent 09-0021), and anti-actin (Santa Cruz sc-47778).

### Co-Immunoprecipitation

Secondary MEFs (10,000 cells/cm²) were plated onto a gelatin-coated 10-cm dish and cultured in reprogramming medium with 1 μg/ml doxycycline for 2 days. Cells were lysed with 500 μL ice-cold IP buffer (50 mM Tris-HCl pH 7.4, 150 mM NaCl, 1% TritonX-100, 0.1% NP-40, and 1.5 mM EDTA) on ice for 20 minutes. Protein A dynabead slurry (20 μL, Life Sciences Technologies, 10001D) was used for each IP test. Elute target and co-IP proteins with SDS sample buffer for direct western detection.

### Immunofluorescent Staining and Image Analysis

Cells were washed three times with DPBS and fixed with 4% PFA for 30 minutes at 4° C. Donkey serum (10% in DPBS) with 1% BSA was used for blocking for 1 hour at 4° C. Triton-X100 (0.3%) was added during blocking when staining of nuclei-located proteins. Antibodies were diluted in DPBS with 1% BSA. The following primary antibodies were used for staining: anti-Sox2 (Millipore, AB5603; Abcam, ab79351), anti-Klf4 (Stemgent, 09-0021; R&D, AF3158), anti-c-Myc (epitomics, 1472-1), anti-Nanog (Abcam, 80892), and anti-SSEA-1 (Stemgent, 09-0095).

Single visual field imaging was performed with fluorescent microscope (IX83, Olympus); Images were taken and analyzed using CellSens Dimension. For multiple visual fields imaging and analysis, cell culture plates were scanned using automated microscope (Lionheart FX, BioTek). Images were concatenative synthesized and analyzed using Gen5 Software.

### RNA Extraction

For cultured cells, samples were lysed, and total RNA was extracted with RNeasy Plus mini kit (Qiagen, 74136) with QiaShredder (Qiagen, 79656) according to the manufacture's instruction. For sorted cells, samples at the indicated time points were collected and lysed in TRIzol™ Reagent (Invitrogen, 15596026). Total RNA was extracted as the following procedures. Linear acrylamide (Thermo Fisher Scientific, AM9520) was added to lysed cell samples for enhancing the precipitation of RNA. Chloroform was then added, and the mixtures was shaken vigorously with lysed samples to extract RNA. After centrifugation, RNA dissolved in aqueous phase was carefully transferred into an RNase free tube and mixed intensively with 1 volume of isopropanol (Sigma Aldrich). Samples were then placed at −20° C. overnight to precipitate RNA. On the next day, isopropanol was carefully removed after centrifugation and RNA was pelleted at bottom of the tube. The RNA pellet was then washed with 75% ethanol to eliminate possible residual traces of guanidinium. Ethanol was then removed after centrifugation by pipet tip and 10 minutes of air dry. Finally, total RNA was dissolved in 20 μl of nuclease-free water by pipetting up and down several times if necessary.

### Quantitative PCR

To test the gene expression level, total RNA was used for qPCR experiments. Genomic DNA elimination and reverse transcription were performed using the iScript cDNA synthesis kit (Bio-Rad), and qPCR was performed with iQ™ SYBR Green Supermix (Bio-Rad) on CFX384 Real-Time PCR System (Bio-Rad). All reactions were done in quadruplicate. All data were statistically analyzed in Prism 7 with the build-in analysis methods.

### RNA Sequencing of Reprogramming MEFs and NPCs

Total RNA of samples at the indicated times were used for sequencing. Sequencing libraries were generated using NEBNext® Ultra™ RNA Library Prep Kit for Illumina® (NEB #E7530L), according to the manufacturer's instructions. A total amount of 2 μg RNA per sample was used as input material for library preparation. The library fragments were purified with QiaQuick PCR kits (Qiagen, 28106), quality-controlled by Agilent Bioanalyzer 2100 system (Agilent Technologies, CA, USA) and quantified by qPCR. Libraries were then sequenced using Illumina HiSeq 2500 platform and 150 bp paired-end (PE150) reads were generated.

### Chromatin Immunoprecipitation

All ChIP experiments were performed with EZ-ChIP Chromatin Immunoprecipitation kit (Millipore, 17-371), following the protocol provided with the kit with minor modifications. Briefly, day 0 or days 2 reprogramming cells (˜1.0×10⁷) in a 15-cm dish were crosslinked with 0.55 ml of 37% formaldehyde to 20 ml of growth medium. 1 ml of 2.5 M glycine (20×) was added to quench the unreacted formaldehyde. Cells in each 15-cm plate were collected and resuspended in 830 μl of lysis buffer. Genomic DNA was then sheared to a length of 100-500 bp on Covaris S220 Sonicator with optimized conditions. For Sox2 or Klf4 ChIP, 1.0×10⁷ reprogramming cells and 10 μg of antibody were used for each experiment, and for H3K27ac ChIP, 5.0×10⁶ reprogramming cells and 2 μg of antibody were used. Finally, DNA fragments were recovered with NucleoSpin Gel and PCR Clean-up kit (MAGHEREY-NAGEL, 740609) and used for either qPCR or library preparation. The primary antibodies used are as follows: anti-Sox2 (Millipore, AB5603), anti-Klf4 (R&D, AF3158), and anti-H3K27ac (Abcam, ab4729).

### Preparation of DNA Library for Sequencing

Sequencing libraries were generated with NEBNext® Ultra™ II DNA Library Prep Kit for Illumina (E7645S), according to the manufacturer's instructions. Briefly, 4 ng of ChIP DNA and 40 ng of Input DNA were used for library preparation. NEBNext Multiplex Oligos for Illumina (Set 1, NEB #E7335; Set 2, NEB #E7500) were used for PCR amplification of adaptor-ligated DNA. Libraries were purified with SPRIselect® Reagent Kit (Beckman Coulter, Inc. #B23317), quality controlled by Bioanalyzer 2100, and quantified by qPCR. Sequencing was performed on Illumina NextSeq 550AR using single end 50-bp reads.

### Statistical Analysis

Statistical analyses were performed in GraphPad Prism 7. Significance and the value of n were calculated with the indicated methods in each figure legend. The data are presented as the mean±SD. *p<0.05; **p<0.01; ns, non-significant.

### Alignment and Processing of RNA-Seq Data

Before alignment, low quality reads and those containing adapter or poly-N were removed using FastQC. The remaining reads were mapped to the assembly mm9 genome using the default parameters in STAR (2.5.1b) aligner.

### Clustering of RNA-Seq Data

To clustering samples at different reprogramming time point, Manhattan method was used to find the distance and then hierarchical clustering was applied using hclust.

### Differentially Expressed Genes Analysis

Differentially expressed genes (DEGs) of two groups was performed using the DESeq2 R package (1.10.1). DESeq2 provides statistical routines for determining differential expression from digital gene expression data using a model based on the negative binomial distribution. The resulting P-values were adjusted using the Benjamini and Hochberg's approach to control the false discovery rate. Genes with an adjusted P-value <0.05 found by DESeq2 were designated as differentially expressed.

### Principal Component Analysis

Principal Components Analysis (PCA) was performed in R with R packages gmodels (2.16.2). Fast.prcomp was used for efficient computation of principal components and singular value decompositions.

### Ontology Annotation

Gene ontology (GO) enrichment of DEGs during reprogramming was calculated using the DAVID 6.8 functional annotation bioinformatics tool (see website at david.ncifcrf.gov). Terms that had a P-value <0.05 were defined as significantly enriched.

### Correlation Analysis of SKM Samples

The correlation of all RNA sequencing data between MEF or NPC samples at different reprogramming times and was analyzed in R using pheatmap (1.0.10). The correlation of 112 pluripotency-associated genes between reprogramming NPCs and MEFs were analyzed using corrplot (0.84).

### Alignment and Processing of ChIP-Seq Data

Alignment of the ChIP-seq reads was done using Bowtie2 with mouse genome build mm9, the result was then filtered by MAPQ (0.1.19) scores with smtools to only keep reads with MAPQ larger than 10 (Langmead et al., 2009). To identify regions of ChIP-Seq enrichment over background, peak callings were performed by MACS2 (2.1.0), using the corresponding input DNA as control for each sample (Zhang et al., 2008). Default parameters in MACS were used. The number of reads per million mapped reads (RPM) was calculated in each peak and the corresponding input control of that peak.

### Motif Analysis

The fasta sequences for the peak regions called from MACS were collected and used as input for the motif finding algorithm MEME-Chip (maximum motif width=30, assuming any number of motifs per sequence) (Machanick and Bailey, 2011).

### Peak Distribution Analysis

Genomic Regions Enrichment of Annotations Tool (GREAT) was used to analyze the peak distribution (McLean et al., 2010). For each peak, the smallest distance was calculated between the peak and the nearby Transcription Start Site (TSS) of genes (negative distance for peaks upstream of TSS). The distributions of distance for the peaks from different samples were compared. Bedtools was used to intersect the peaks from Sox2 and Klf4 to identify the colocalized (Sox_Klf) peaks, Sox_solo, and Klf_solo peaks.

### Comparison of Binding Profiles

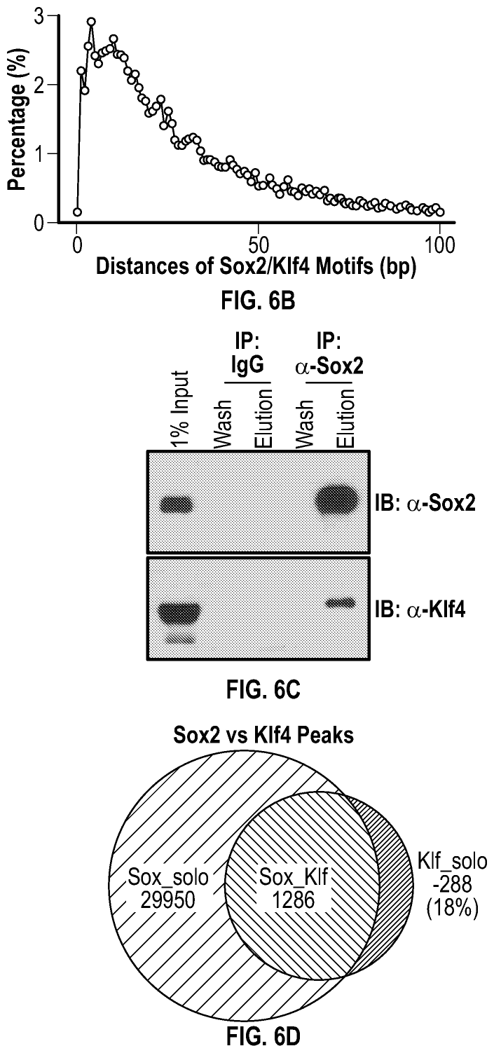

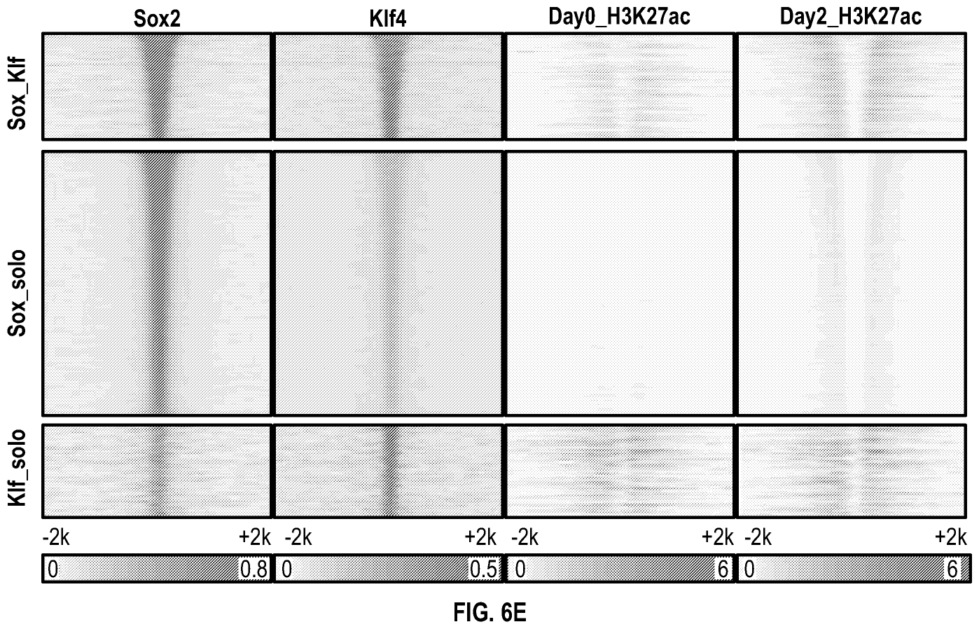

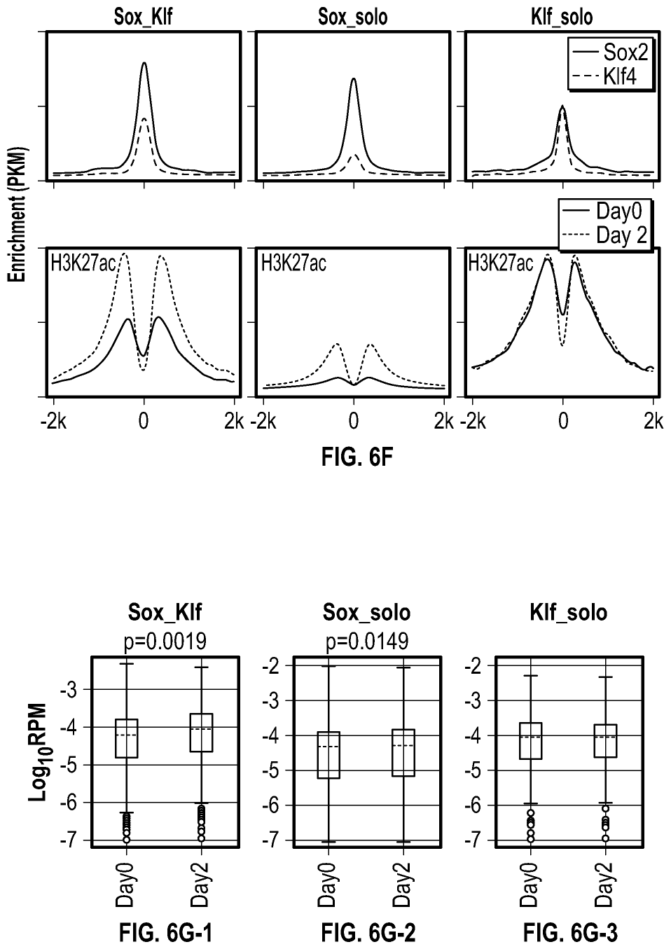

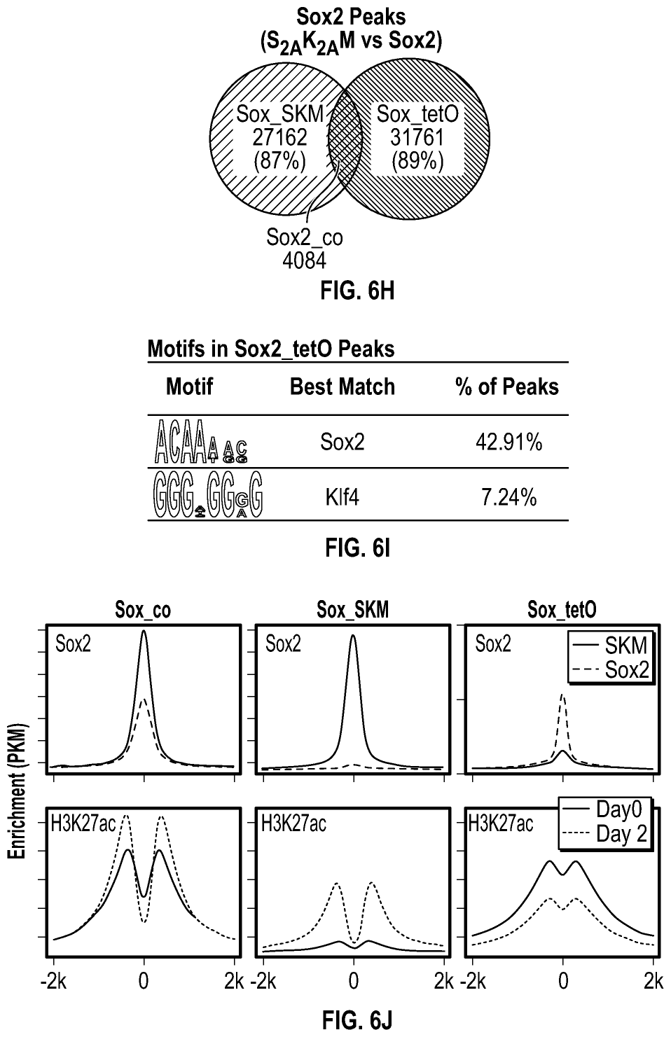

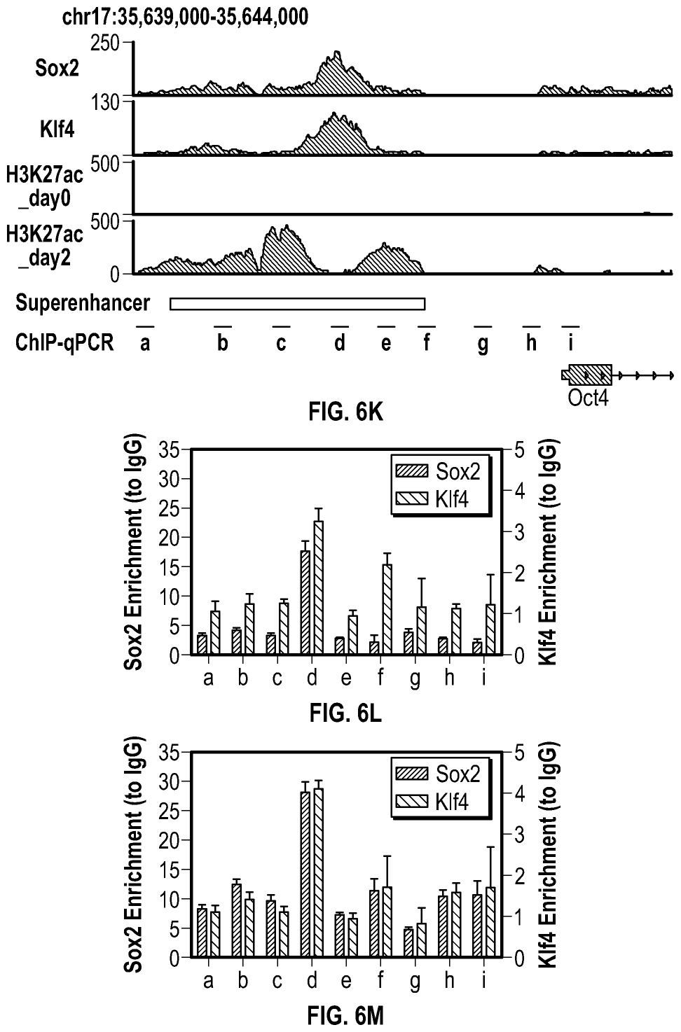

Sox2, Klf4, and H3K27 acetylation ChIP-seq signals of the Sox_Klf, Sox_solo and Klf_solo peaks were analyzed and quantitatively measured, sorted by the intensity of Sox2 in Sox_Klf and Sox_solo and by the intensity of Klf4 in Klf_solo. Ngsplots was used to create the heatmap and average profile plot in FIGS. 6E and 6F around the center of three groups of peaks (Shen et al., 2014).

### Sox2 Target Genes Analysis

Genes with the TSS within +/−5 kb of the Sox_Klf, Sox_solo and Klf_solo peaks were identified. A Mann-Whitney U test was performed to measure the statistical significance of the difference between the normalized reads of each group of genes.

### Binding Profile Analysis

The enrichment of binding peaks of Sox2, Klf4 and H3K27 acetylation in pluripotency-associated regions was visualized in IGV (2.4.10). ChIP-qPCR was further conducted for detection of Sox2 and Klf4 binding property from the first exon to the distal enhancer of Oct4. Primers used for qPCR are listed in Table 2.

TABLE 2
PCR primer sequences

| Gene Name | | | |
|---|---|---|---|
| or Target | | | |
| Location | | Sequence | Note |
| Oct4 | Forward | ACATCGCCAATCAGCTTGG | qPCR for |
| | | (SEQ ID NO: 36) | gene |
| | Reverse | AGAACCATACTCGAACCACATCC | expression |
| | | (SEQ ID NO: 37) | |
| Sox2 | Forward | ACAGATGCAACCGATGCACC | |
| | | (SEQ ID NO: 38) | |
| | Reverse | TGGAGTTGTACTGCAGGGCG | |
| | | (SEQ ID NO: 39) | |
| Klf4 | Forward | GCACACCTGCGAACTCACAC | |
| | | (SEQ ID NO: 40) | |
| | Reverse | CCGTCCCAGTCACAGTGGTAA | |
| | | (SEQ ID NO: 41) | |
| c-Myc | Forward | .CCACCAGCAGCGACTCTGA | |
| | | (SEQ ID NO: 42) | |
| | Reverse | TGCCTCTTCTCCACAGACACC | |
| | | (SEQ ID NO: 43) | |
| Nr5a2 | Forward | ATGGGAAGGAAGGGACAATC | |
| | | (SEQ ID NO: 44) | |
| | Reverse | ATACAAACTCCCGCTGATCG | |
| | | (SEQ ID NO: 45) | |
| Nanog | Forward | CCTCCAGCAGATGCAAGAACTC | |
| | | (SEQ ID NO: 46) | |
| | Reverse | CTTCAACCACTGGTTTTTCTGCC | |
| | | (SEQ ID NO: 47) | |
| Esrrb | Forward | CTCGCCAACTCAGATTCGAT | |
| | | (SEQ ID NO: 48) | |
| | Reverse | AGAAGTGTTGCACGGCTTTG | |
| | | (SEQ ID NO: 49) | |
| Fgf4 | Forward | CGTGGTGAGCATCTTCGGAGTGG | |
| | | (SEQ ID NO: 50) | |
| | Reverse | CCTTCTTGGTCCGCCCGTTCTTA | |
| | | (SEQ ID NO: 51) | |
| Tet1 | Forward | TCTCACTCATGTTGCGGGACCC | |
| | | (SEQ ID NO: 52) | |
| | Reverse | CGTCGGAGTTGAAATGGGCGAA | |
| | | (SEQ ID NO: 53) | |
| Utf1 | Forward | TGTCCCGGTGACTACGTCT | |
| | | (SEQ ID NO: 54) | |
| | Reverse | CCCAGAAGTAGCTCCGTCTCT | |
| | | (SEQ ID NO: 55) | |
| Rex1 | Forward | TATGACTCACTTCCAGGGGG | |
| | | (SEQ ID NO: 56) | |
| | Reverse | AGAAGAAAGCAGGATCGCCT | |
| | | (SEQ ID NO: 57) | |
| Actin | Forward | ATGGAGGGGAATACAGCCC | |
| | | (SEQ ID NO: 58) | |
| | Reverse | TTCTTTGCAGCTCCTTCGTT | |
| | | (SEQ ID NO: 59) | |
| Zfp296 | Forward | TCATCGCTTTCATGGATCACA | |
| | | (SEQ ID NO: 60) | |
| | Reverse | ACAGCAACTTCCAAGGACTAG | |
| | | (SEQ ID NO: 61) | |
| Lin28a | Forward | GAAGAGATCCACAGCCCTG | |
| | | (SEQ ID NO: 62) | |
| | Reverse | CCAAAGAATAACCCTGACTCCTG | |
| | | (SEQ ID NO: 63) | |
| Lin28b | Forward | GAGTCAATACGGGTAACAGGC | |
| | | (SEQ ID NO: 64) | |
| | Reverse | TTCTCGCACAGTCCACATC | |
| | | (SEQ ID NO: 65) | |
| Cdh1 | Forward | AACAACTGCATGAAGGCGGGAATC | |
| | | (SEQ ID NO: 66) | |
| | Reverse | CCTGTGCAGCTGGCTCAAATCAAA | |
| | | (SEQ ID NO: 67) | |
| EpCAM | Forward | GCTGGCAACAAGTTGCTCTCTGAA | |
| | | (SEQ ID NO: 68) | |
| | Reverse | CGTTGCACTGCTTGGCTTTGAAGA | |
| | | (SEQ ID NO: 69) | |
| Krt8 | Forward | TCCATCAGGGTGACTCAGAAA | |
| | | (SEQ ID NO: 70) | |
| | Reverse | CCAGCTTCAAGGGGCTCAA | |
| | | (SEQ ID NO: 71) | |
| Ocln | Forward | CCTCCAATGGCAAAGTGAATGGCA | |
| | | (SEQ ID NO: 72) | |
| | Reverse | TGTTTCATAGTGGTCAGGGTCCGT | |
| | | (SEQ ID NO: 73) | |
| Oct4 | Forward | TGAATGAGTGATGTCGTGGG | ChIP-qPCR |
| upstream | | (SEQ ID NO: 74) | for 4.5-kb |
| a | Reverse | CTTCTGATCCTCTTGCCTTCC | sequence |
| | | (SEQ ID NO: 75) | upstream of |
| | | | Oct4 gene |
| Oct4 | Forward | CATGTCGCTGAAACTCCTCA | |
| upstream | | (SEQ ID NO: 76) | |
| b | Reverse | AATGGACTCACGGAGGACAC | |
| | | (SEQ ID NO: 77) | |
| Oct4 | Forward | TGGCCTGGAACTCAGAAATC | |
| upstream | | (SEQ ID NO: 78) | |
| c | Reverse | TGCCTCCTGGGTCTTAGAAA | |
| | | (SEQ ID NO: 79) | |
| Oct4 | Forward | GACGGCAGATGCATAACAAA | |
| upstream | | (SEQ ID NO: 80) | |
| d | Reverse | AAGGAAGGGCTAGGACGAGA | |
| | | (SEQ ID NO: 81) | |
| Oct4 | Forward | CCCAGGCTCAGAACTCTGTC | |
| upstream | | (SEQ ID NO: 82) | |
| e | Reverse | TGCTCCTACACCATGCTCTG | |
| | | (SEQ ID NO: 83) | |
| Oct4 | Forward | TCCTCCTAATCCCGTCTCCT | |
| upstream | | (SEQ ID NO: 84) | |
| f | Reverse | ATACCCTGCTTCCCTTCCTC | |
| | | (SEQ ID NO: 85) | |
| Oct4 | Forward | CTGGGGACATATCTGGTTGG | |
| upstream | | (SEQ ID NO: 86) | |
| g | Reverse | CCCAGTATTTCAGCCCATGT | |
| | | (SEQ ID NO: 87) | |
| Oct4 | Forward | TTGAAAATGAAGGCCTCCTG | |
| upstream | | (SEQ ID NO: 88) | |
| h | Reverse | AGCGCTATCTGCCTGTGTCT | |
| | | (SEQ ID NO: 89) | |
| Oct4 | Forward | TAGGTGAGCCGTCTTTCCAC | |
| upstream | | (SEQ ID NO: 90) | |
| i | Reverse | GCTTAGCCAGGTTCGAGGAT | |
| | | (SEQ ID NO: 91) | |
| Oct4 | Forward | GAGGATTGGAGGTGTAATG | First |
| Promoter | | GTTGTT (SEQ ID NO: 92) | round of |
| | Reverse | CTACTACCCATCACCCCCACTA | PCR for |
| | | (SEQ ID NO: 93) | Bisulfite |
| Oct4 | Forward | CAAGCTTTGGGTTGAAATATTGG | Second |
| Promoter | | GTTTATTT (SEQ ID NO: 94) | round of |
| | Reverse | CGGATCCCTAAAACCAAATATCC | PCR for |
| | | AACCATA (SEQ ID NO: 95) | Bisulfite |

### Data and Code Availability

The accession number for the RNA-seq data and ChIP-seq data is NCBI GEO: GSE98280.

### Example 2: S_{2A}K_{2A}M Reprograms Fibroblasts into iPSCs

This Example describes use of polycistronic expression cassettes to precisely and conveniently control the stoichiometry of multiple factors at the single-cell level.

Polycistronic cassettes were constructed with 2A peptide cleavage sequences (de Felipe et al., 2006) between the segments encoding the reprogramming factors (e.g., Oct4, Klf4, Sox2, and/or c-Myc). Various combinations of two-pioneer factors were initially tested, and c-Myc (M) was included in all combinations because of its purported function in enhancing reprogramming efficiency through transcriptional amplification (Lin et al., 2012; Nie et al., 2012).

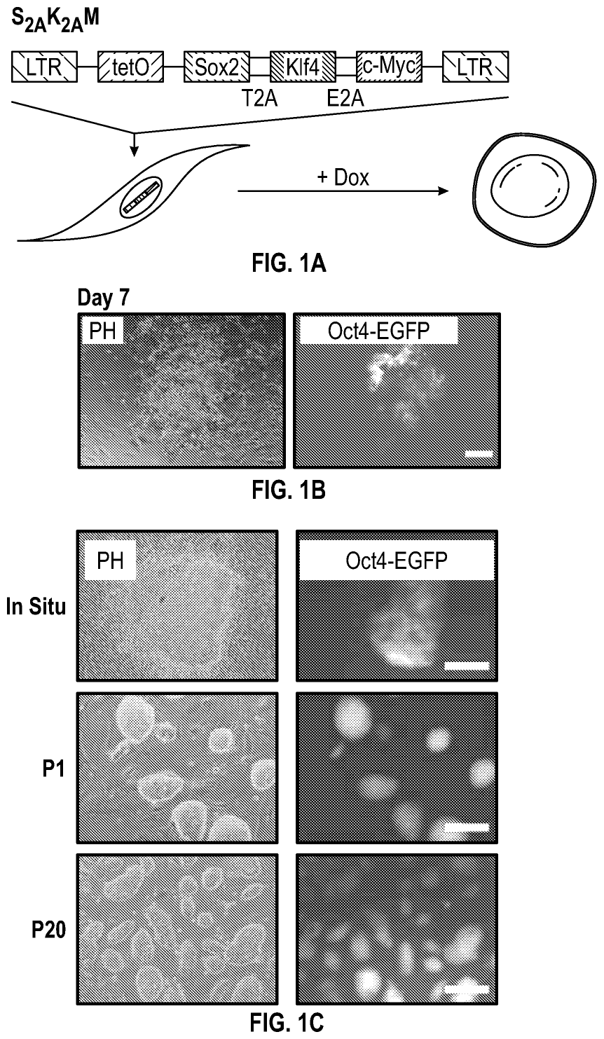

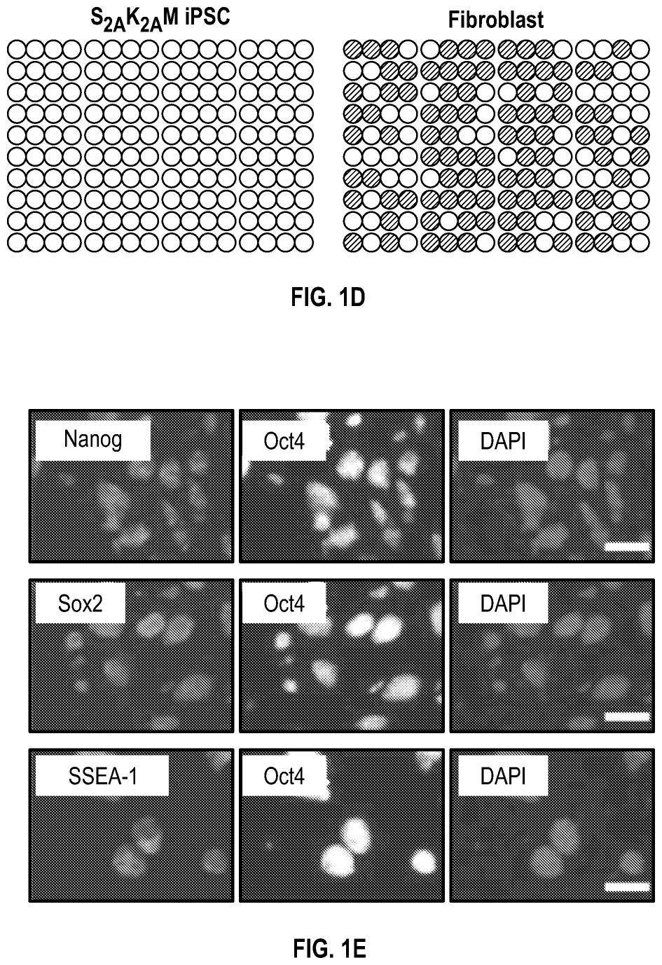

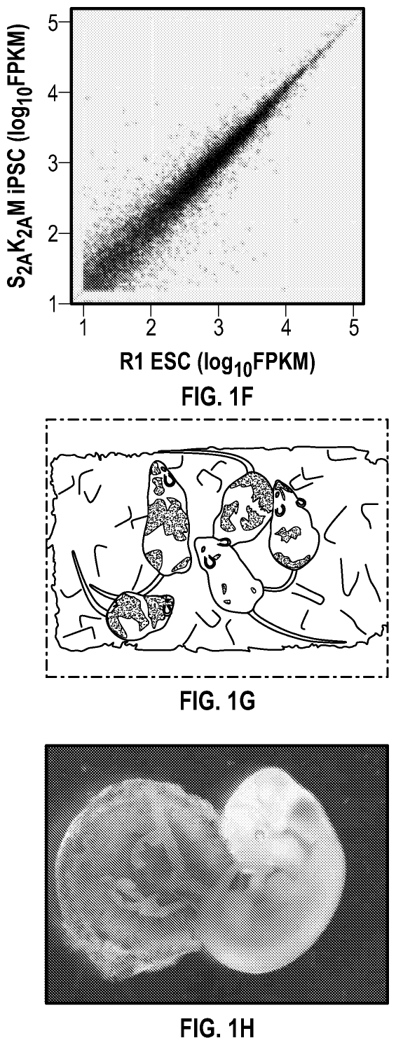

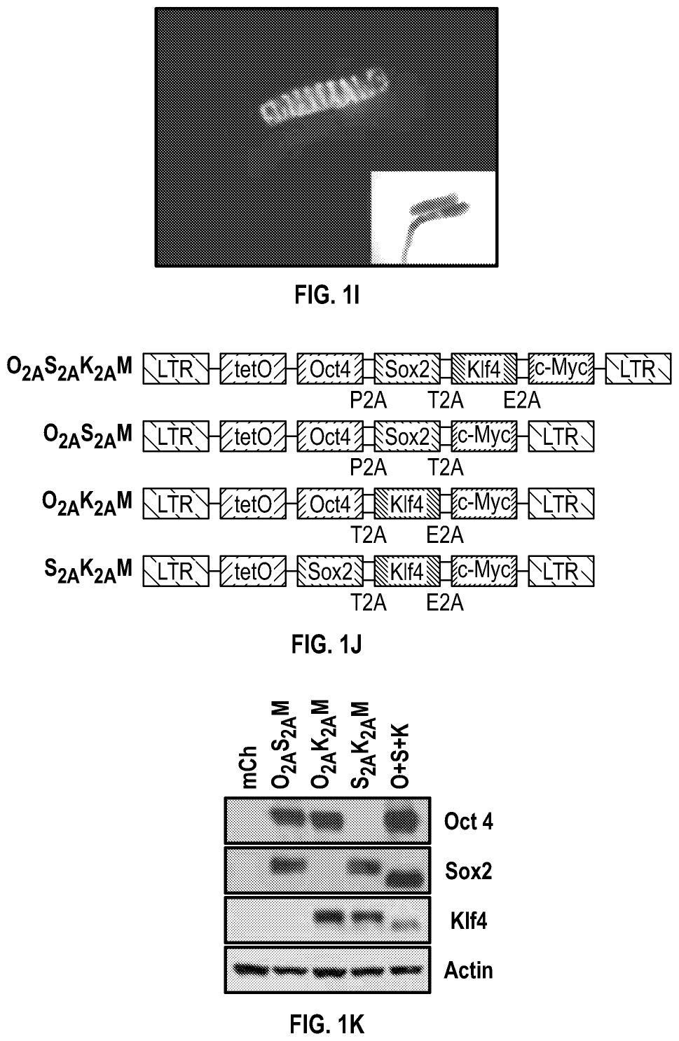

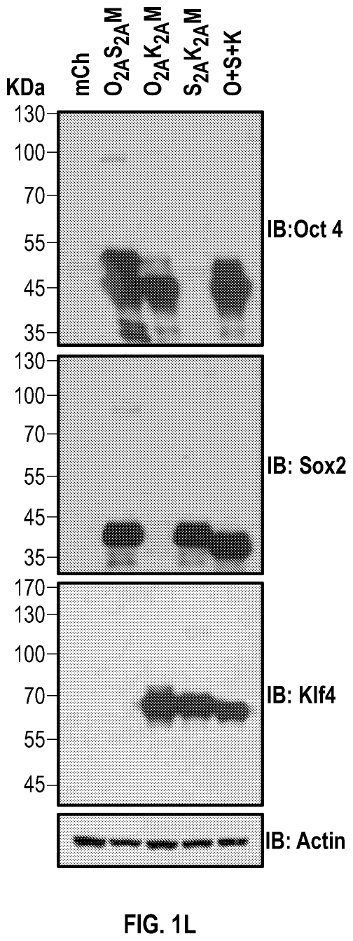

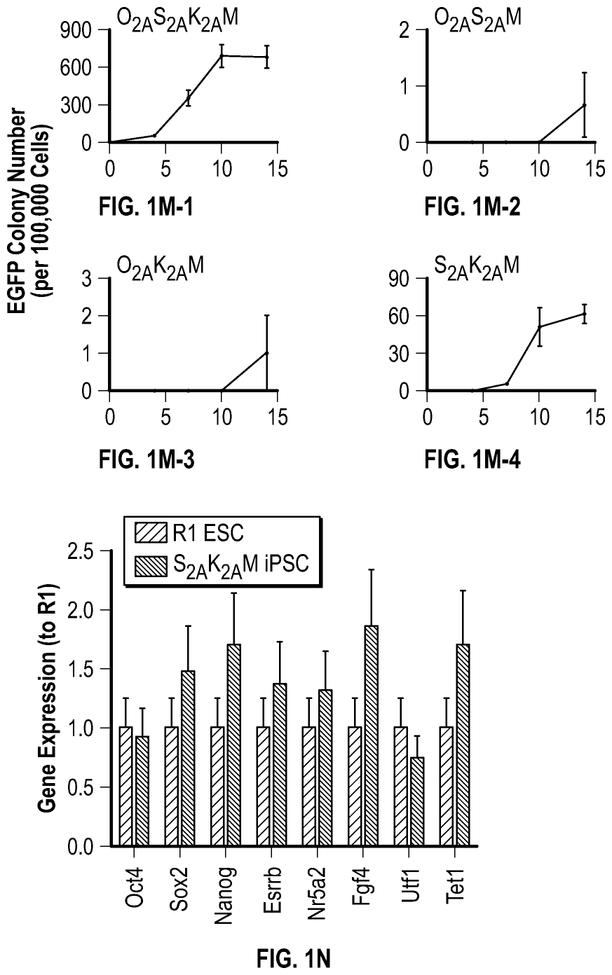

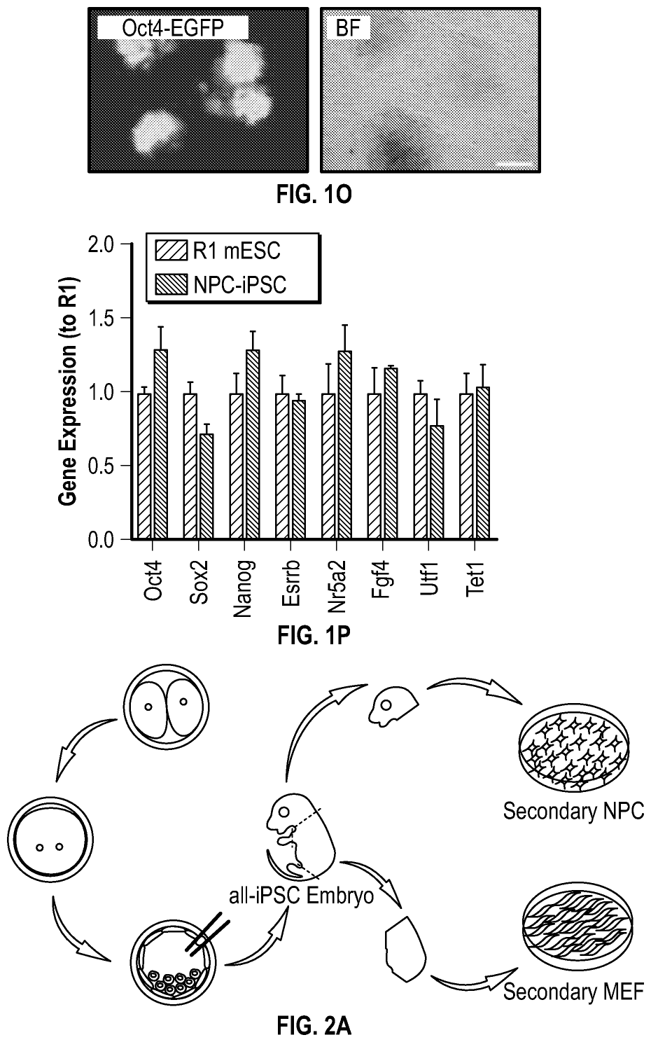

Thus, polycistronic Oct4, Sox2, and c-Myc (O_{2A}S_{2A}M), Oct4, Klf4, and c-Myc (O_{2A}K_{2A}M), and Sox2, Klf4, and c-Myc (S_{2A}K_{2A}M), were derived from a previous O_{2A}S_{2A}K_{2A}M plasmid (Carey et al., 2009). These cassettes were transduced into mouse embryonic fibroblasts (MEFs) (FIGS. 1A and 1J), and protein expression was assessed by western blots, confirming high efficiency of the polycistronic peptide processing (FIGS. 1K-1L).

Three combinations were first tested for their capacity to induce reprogramming in OG2 MEFs following a widely used method (Takahashi and Yamanaka, 2006). OG2 MEFs harbor an EGFP reporter under control of the endogenous Oct4 promoter, so the EGFP signal can be used as a mark of reprogramming efficiency (Szabo et al., 2002). During the 2-week reprogramming, EGFP-positive colonies were counted on days 4, 7, 10, and 14. Surprisingly, EGFP-positive colonies were observed by day 7 under the S_{2A}K_{2A}M condition, and on day 14, about 60 EGFP-positive colonies were produced per 100,000 starting MEFs (0.06%) (FIGS. 1B & 1M). This efficiency was greater than that observed in the O_{2A}S_{2A}M and O_{2A}K_{2A}M conditions, although and still 10-fold less efficient than the O_{2A}S_{2A}K_{2A}M condition (FIG. 1M).

S_{2A}K_{2A}M generated typical iPSC-like colonies, and iPSC lines were derived from these colonies. When these lines were passaged in ESC medium, they formed ESC-like domed colonies and were Oct4-EGFP positive, which remained unchanged even after 20 passages (FIG. 1C). Consistent with this stable marker expression, bisulfite sequencing indicated that the Oct4 promoter was completely demethylated in these cells (FIG. 1D). Immunofluorescence analysis showed that these cells were positive for Nanog, Sox2, and SSEA1, and the global gene expression was very similar to the R1 mouse ESC line (FIGS. 1E, 1F & 1N). These data suggested that a pluripotency network had been established in the S_{2A}K_{2A}M iPSCs.

The functional pluripotency of these lines was then tested by examining their capacity to form teratomas and chimeras. S_{2A}K_{2A}M iPSCs were able to form teratomas that contained all three germ layers and were successfully used to generate chimeric mice (FIG. 1G). Pluripotency of these lines was then tested using the most stringent method, the tetraploid complementation assay (4N). Normal live embryos were recovered on E13.5, suggesting the proper in vivo differentiation of the iPSCs into all the tissues. The EGFP signal could be observed in the gonadal regions of the embryos (FIGS. 1H and 1I), demonstrating the successful transmission to the germ line.

### Example 3: S_{2A}K_{2A}M Reprograms Multiple Differentiated Cell Types into Pluripotency

This Example describes experiments illustrating the capacity of S_{2A}K_{2A}M to reprogram different cell types to form pluripotent stem cells.

OG2 neural progenitor cells (NPCs), which expressed the NPC markers Nestin, Sox2, and Pax6 and formed neural spheres were transduced with S_{2A}K_{2A}M and exposed to a similar reprogramming protocol. OG2 MEFs harbor an EGFP reporter under control of the endogenous Oct4 promoter, so the EGFP signal can be used as a mark of reprogramming efficiency (Szabo et al., 2002). EGFP-positive colonies were obtained after 2 weeks, and stable iPSC lines were established (FIGS. 1O-1P), demonstrating that cells from ectoderm can also be reprogrammed by S_{2A}K_{2A}M.

Next, a more differentiated cell type, OG2 adult mouse tail tip fibroblasts (TTFs), was examined. Similarly, following S_{2A}K_{2A}M transduction and the reprogramming protocol, iPSC lines were obtained from the EGFP-positive colonies, and their pluripotent gene expression was not distinguishable from R1 ESCs.

### Example 4: S_{2A}K_{2A}M 2° MEFs and NPCs can be Efficiently Reprogrammed to Pluripotency

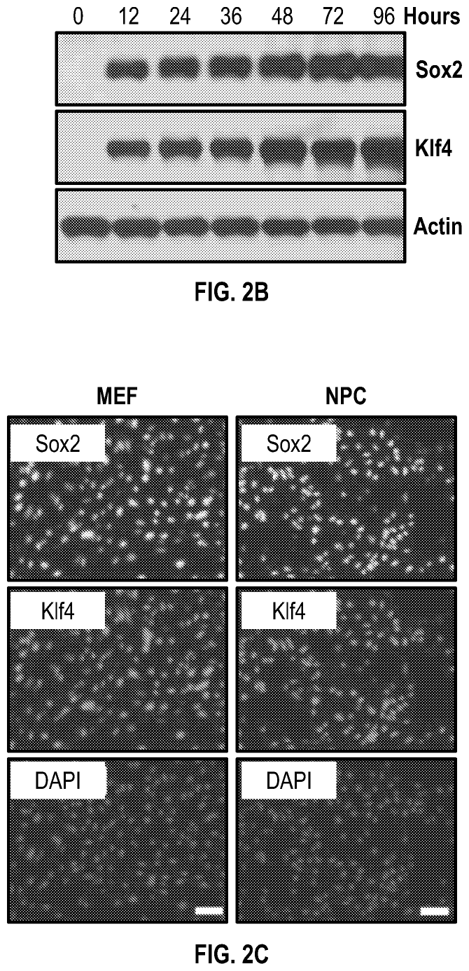

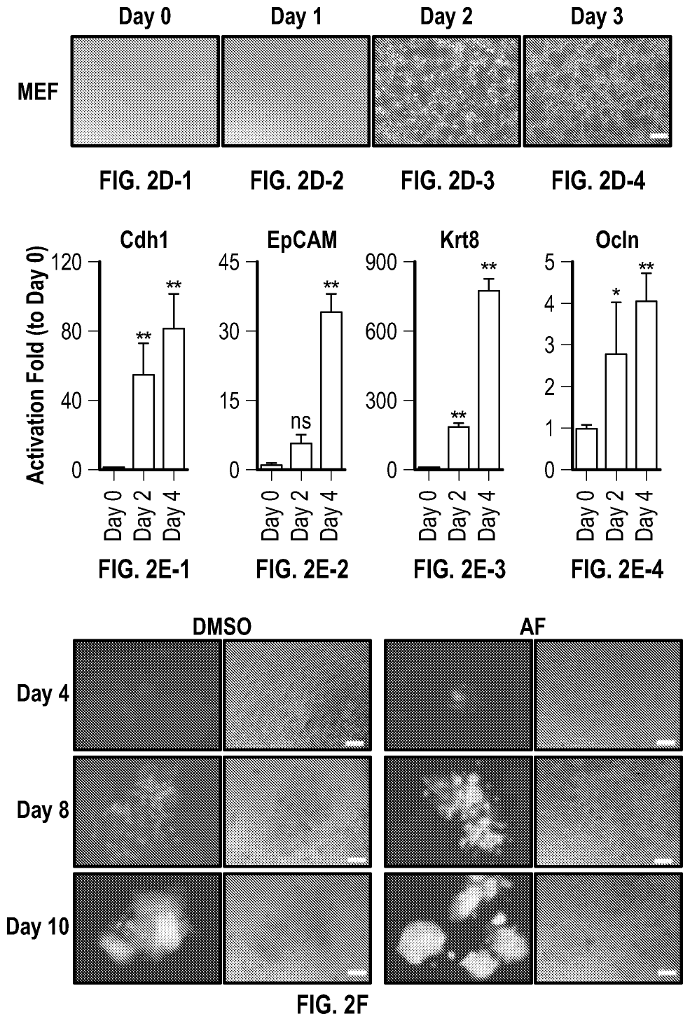

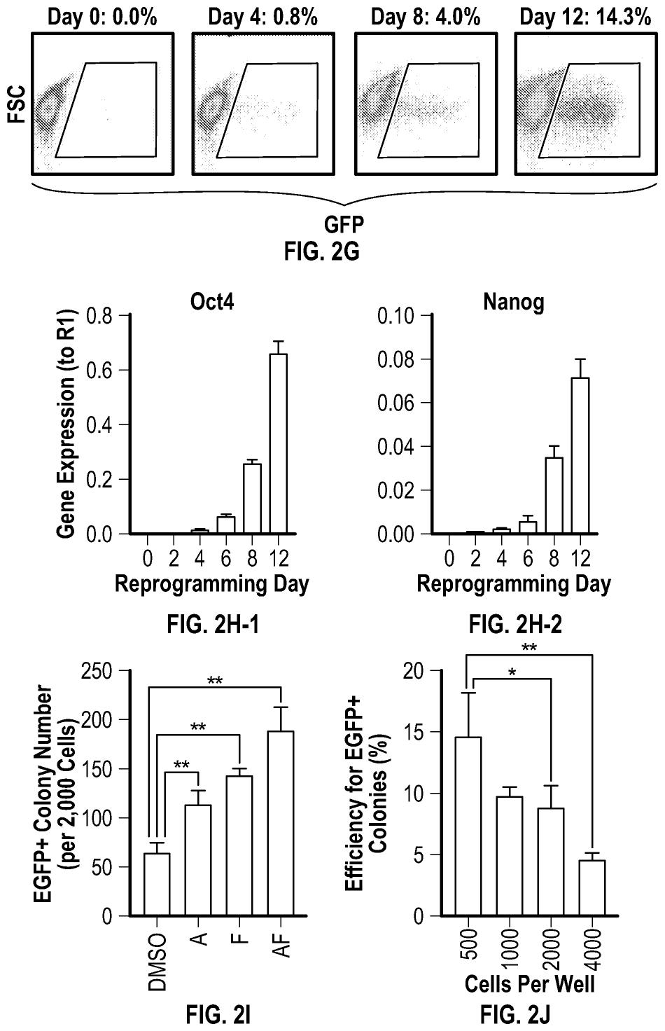

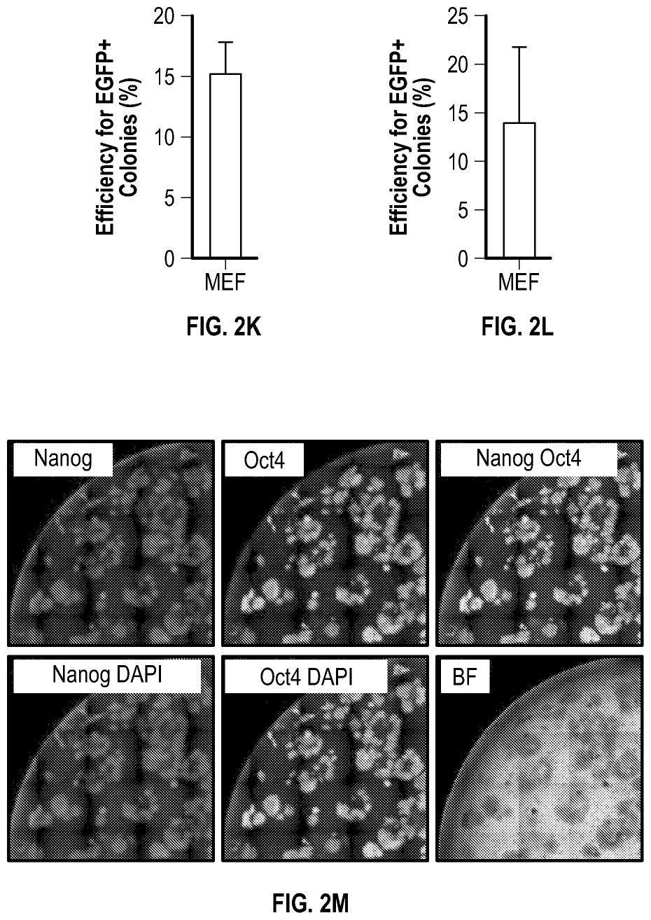

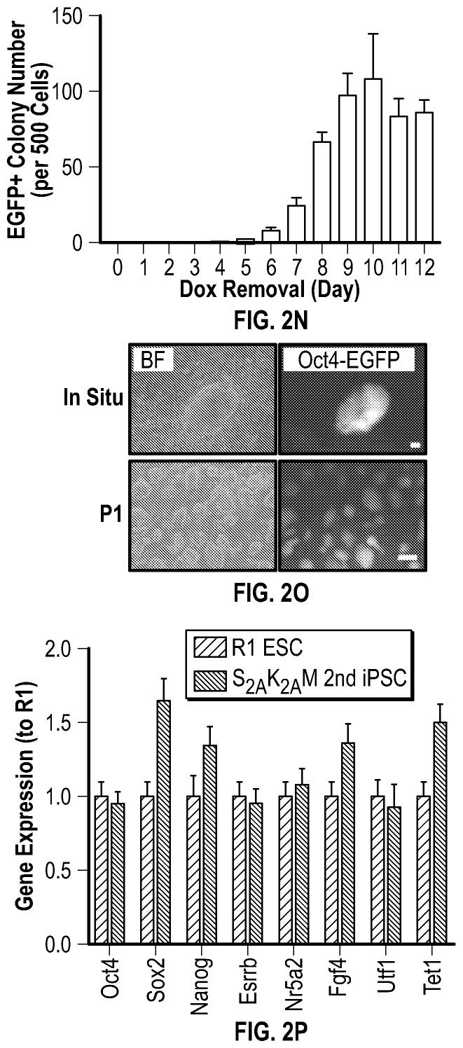

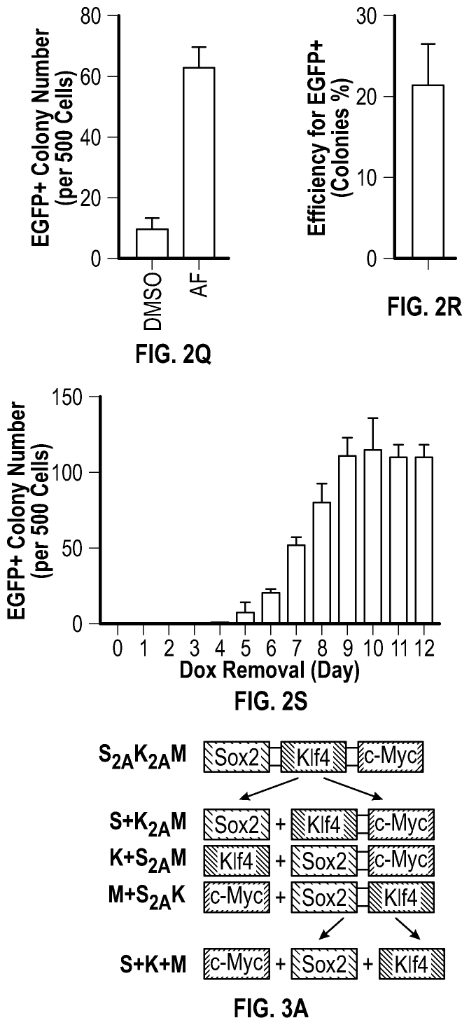

This Example describes S_{2A}K_{2A}M-mediated reprogramming of MEFs and NPCs from embryos generated via the 4N assay with S_{2A}K_{2A}M iPSCs. These embryo derived MEFs and NPCs were referred to as secondary S_{2A}K_{2A}M cells (or S_{2A}K_{2A}M 2° MEFs and NPCs), because these cells were 100% iPSC derived (FIG. 2A).

These 2° MEFs and NPCs responded to doxycycline robustly. After 12 hours of induction, the Sox2 and Klf4 proteins were readily detected (FIG. 2B). The immunostaining showed that 2° MEFs or NPCs universally expressed Sox2 and Klf4 after 24 hours (FIG. 2C), verifying that all cells were derived from the S_{2A}K_{2A}M iPSCs.

The inventors then evaluated whether the 2° MEFs could be reprogrammed. After 2 days, all cells underwent dramatic morphological changes simultaneously, which became more pronounced on day 3 (FIG. 2D). As shown in FIG. 2E, upregulation was observed of mesenchymal-to-epithelial transition (MET) genes, including Cdh1, EpCAM, Krt8, and Ocln. On day 4, EGFP-positive cell clusters were observed, and iPSC-like colonies could be easily identified by day 10 (FIGS. 2F and 2G), consistent with the upregulation of Oct4 and Nanog, although the relatively low level of Nanog on day 12 suggested that these EGFP-positive cells were still not fully reprogrammed (FIG. 2H). With further culturing, 2° iPSC lines were established from these colonies (FIGS. 2O-2P). Reprogramming with 2° NPCs occurred with similar kinetics, except that the EGFP signal was not observed until 2 days later, on day 6.

During the MEF reprogramming, approximately 3% of the cells were reprogrammed to form EGFP-positive colonies (FIG. 2I). This is comparable to the efficiency of OSKM 2° reprogramming observed in another study (2-4%) (Wernig et al., 2008). The inventors also tested if greater efficiency could be achieved by optimizing the culture conditions. First, two small molecules, Forskolin and A83-01, can be used to promote reprogramming though activating cAMP generation while inhibiting TGF-β pathway. When Forskolin and A83-01 were added into the medium, a threefold increase in EGFP-positive colonies was observed (FIG. 2I) with no change in the general reprogramming kinetics (FIG. 2F). Second, the effect of cell density was tested. A higher density of cells in the culture was observed that significantly decreased the reprogramming efficiency (FIG. 2J).

With the optimized conditions, the efficiency of generating EGFP-positive colonies was then precisely calculated. Exact cell numbers were counted before reprogramming, and after 12 days. As shown in FIG. 2K, 15% of the cell population gave rise to EGFP-positive colonies. Importantly, nearly 100% of these EGFP-positive colonies were positive for Nanog after further culturing (FIG. 2M), suggesting the establishment of pluripotency network. As an alternative method the flow cytometry was employed and single cells were seeded into individual wells; from 288 cells seeded, 44 colonies (15.28%) were obtained, and 41 of those (14.24%) were EGFP-positive (FIG. 2L).

Finally, the temporal requirement of exogenous factors for MEF reprogramming was examined. Doxycycline was removed from day 1 to day 12 (FIG. 2N). A minimum of 4 days of induction was required for EGFP-positive colony generation, which coincided with the observation of the earliest EGFP-positive clusters. After day 10, no further increase in colony number was obtained. This suggests that 10 days of induction already reached the maximum number of colonies.

Similar results were also observed for 2° NPCs (FIG. 2Q-2S). Together, these data demonstrate that 2° S_{2A}K_{2A}M MEFs and 2° S_{2A}K_{2A}M NPCs can readily be reprogrammed in a highly efficient manner.

### Example 5: S_{2A}K_{2A}M Optimizes Sox2 and Klf4 Stoichiometry for Reprogramming

This Example illustrates that in addition to providing simultaneous expression of Sox2, Klf4, and c-Myc, the other advantage of S_{2A}K_{2A}M is that the Sox2, Klf4, and c-Myc stoichiometry from the polycistronic cassettes is stable at the single-cell level.

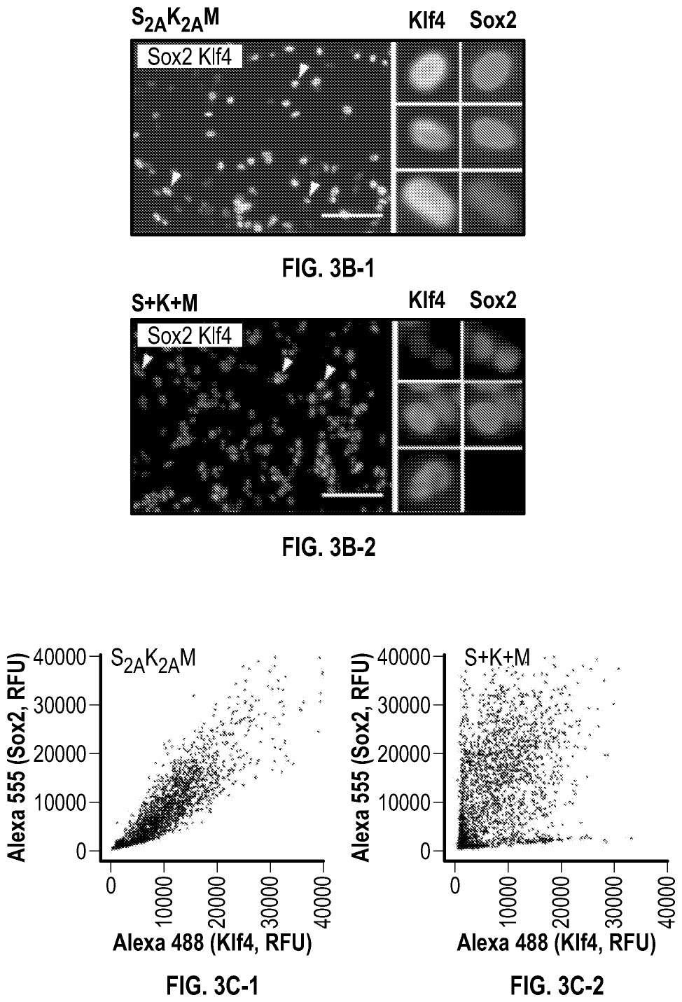

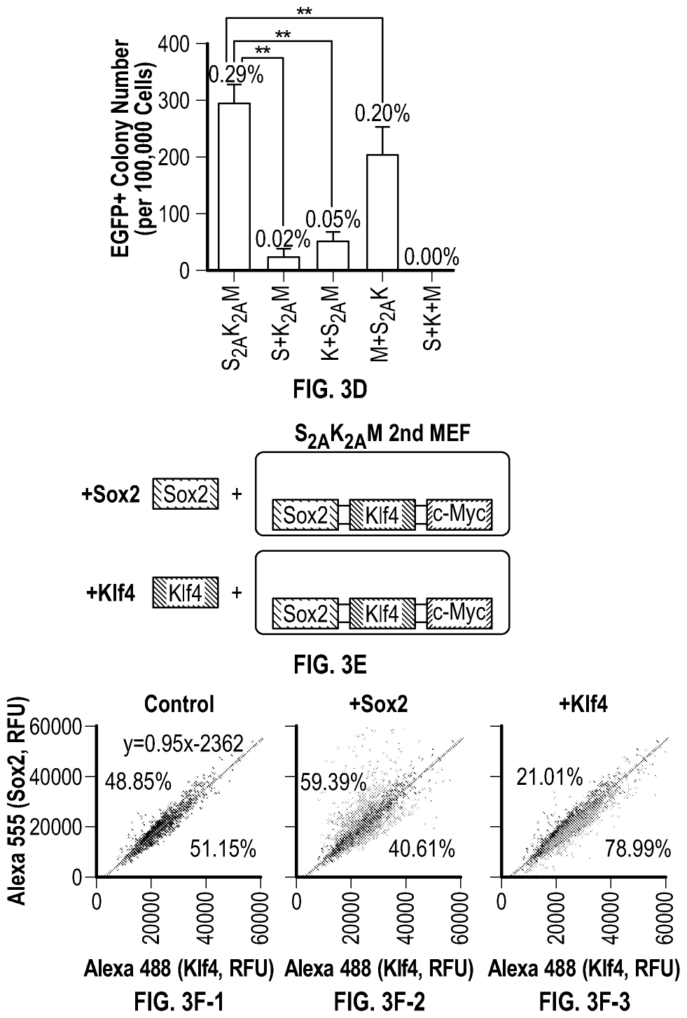

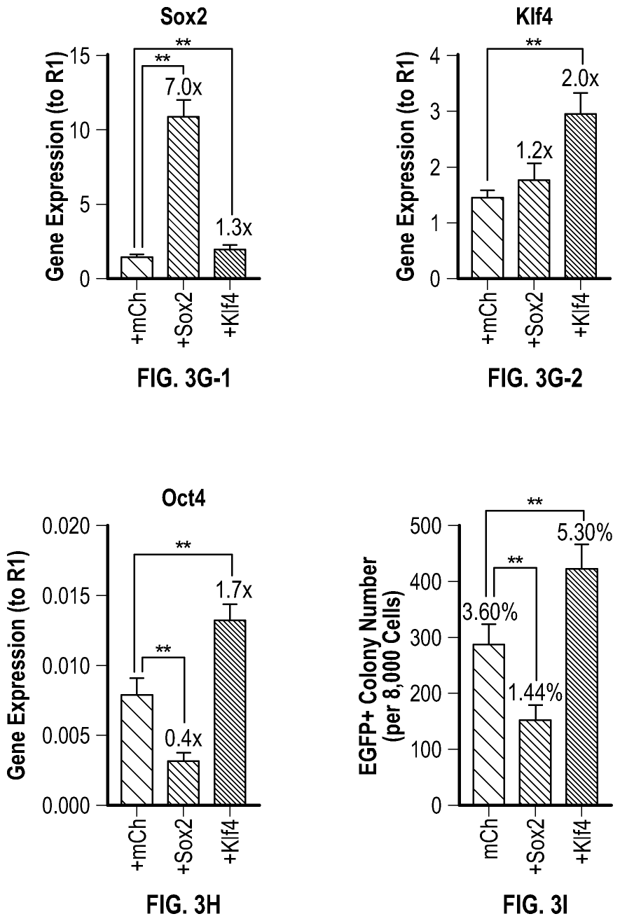

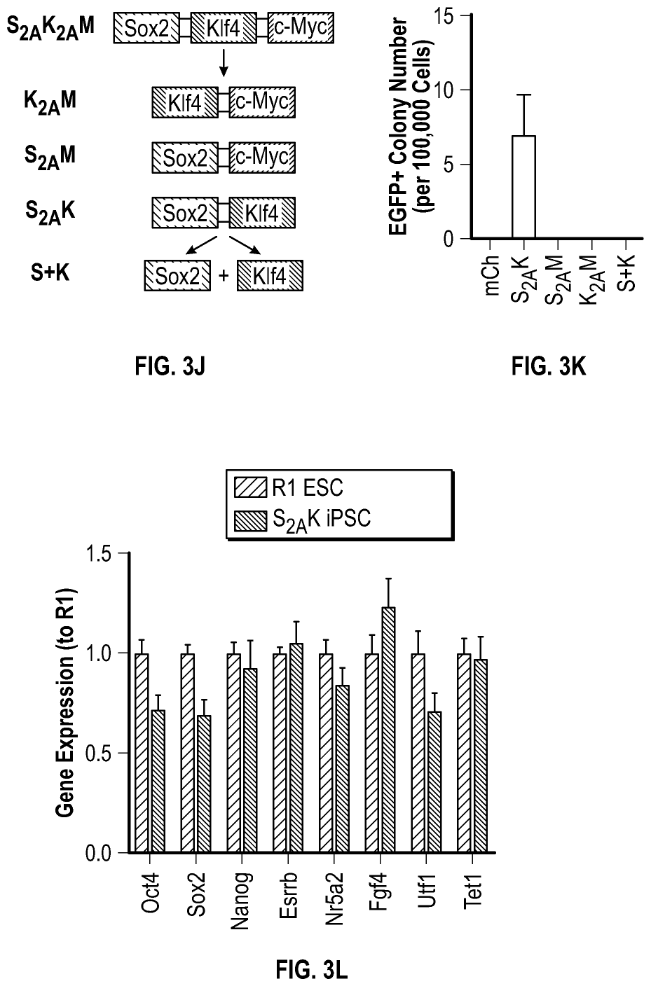

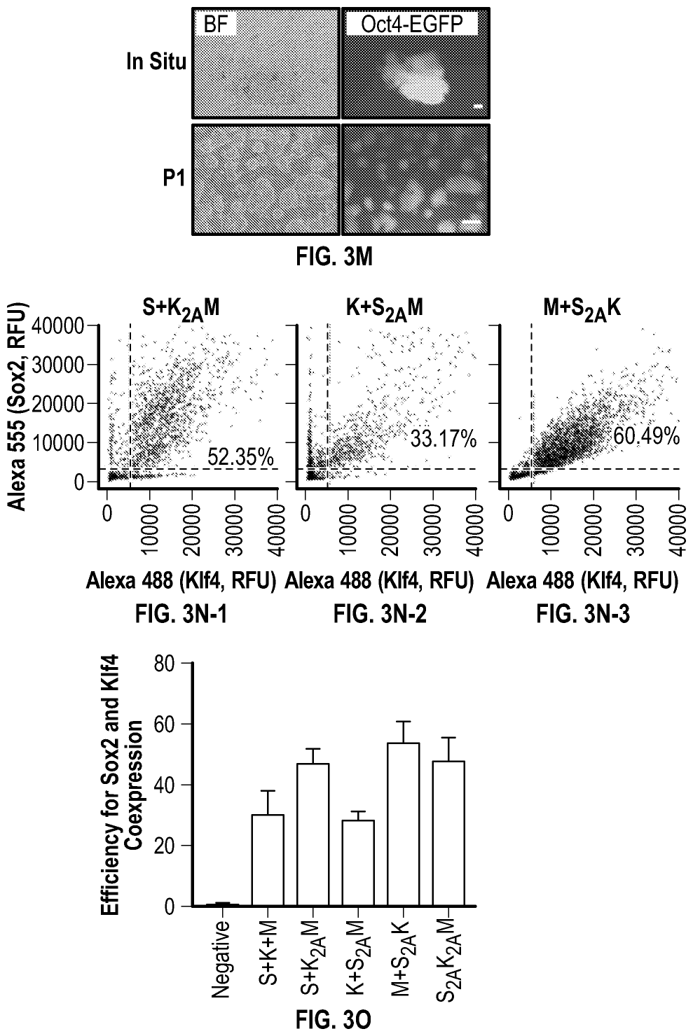

Optimal Sox2, Klf4, and c-Myc stoichiometry was verify by observing the signal intensity of Sox2 and Klf4 as analyzed by immunostaining. In single cells transduced with S_{2A}K_{2A}M, the Sox2 and Klf4 expression signals were generally equivalent, which was in sharp contrast to the mosaic pattern observed in cells transduced with three vectors individually expressing Sox2, Klf4, and c-Myc (S+K+M) (FIGS. 3B-3C).

The effect of disrupting the factor stoichiometry was then tested by moving one factor to a monocistronic cassette, resulting in a combination of monocistronic Sox2 plus polycistronic Klf4 and c-Myc (S+K_{2A}M), monocistronic Klf4 plus polycistronic Sox2 and c-Myc (K+S_{2A}M) and monocistronic c-Myc plus polycistronic Sox2 and Klf4 (M+S_{2A}K) (FIG. 3A). FIG. 3N-1 to 3N-3 illustrate loss of coordinated expression of Sox2 and Klf4 in the S+K_{2A}M and K+S_{2A}M cell types.

The inventors then tested how the disruption of Sox2 and Klf4 stoichiometry would affect the reprogramming outcome. To facilitate the comparison of reprogramming efficiency, viral titrations were adjusted to achieve comparable percentages of cells co-expressing Sox2 and Klf4 in all conditions (FIGS. S3C and S3D). After 16 days of reprogramming, the number of colonies was profoundly lower in conditions when Sox2 and Klf4 were separated, by 90% and 80% in S+K_{2A}M and K+S_{2A}M combinations, respectively than in the S_{2A}K_{2A}M condition, whereas the number of colonies in the M+S_{2A}K was only 30% lower than the control (FIGS. 3N-3O). These results demonstrate that factor stoichiometry, particularly that of Sox2 and Klf4, is critical for S_{2A}K_{2A}M reprogramming.

The inventors further investigated how the stoichiometry of Sox2 and Klf4 affected S_{2A}K_{2A}M reprogramming by manipulating the ratio of the two factors. Sox2 (+Sox2) or Klf4 (+Klf4) were individually overexpressed in 2° MEFs (FIG. 3E). Because S_{2A}K_{2A}M was already expressed in these cells, overexpressing Sox2 or Klf4 would lead to an increased ratio of Sox2/K1f4 in +Sox2 cells and a decreased ratio of Sox2/Klf4 in +Klf4 cells, as verified by single cell fluorescence analysis (FIG. 3F) and qPCR (FIG. 3G). By the end of reprogramming, EGFP-positive colony numbers were smaller for +Sox2_condition and bigger for +Klf4 condition (FIG. 3I). In agreement with these results, on day 4, Oct4 activation was decreased when Sox2 was overexpressed, and it was enhanced when Klf4 was overexpressed (FIG. 3H). These data indicate that a higher Klf4/Sox2 ratio promotes more efficient reprogramming.

The inventors then examined if polycistronic Sox2 and Klf4 was sufficient for iPSC generation without co-expression of c-Myc. The two-factor combinations, S_{2A}K, S_{2A}M, and K_{2A}M, were used for reprogramming (FIG. 3J). Interestingly, EGFP-positive colonies were only obtained in the S_{2A}K condition, and iPSC lines were established (FIGS. 3K-3M). However, when Sox2 and Klf4 were separately expressed from monocistronic plasmids, no EGFP-positive colony was generated. These results again confirm that Sox2 and Klf4 stoichiometry is a factor in reprogramming cells to be pluripotent.

### Example 6: Transcriptional Switches at Day 0/2 and Day 12/iPSC Mark Transitions During 2° MEF and NPC Reprogramming

This Example describes experiments designed to understand how the transcriptional network changed from distinct differentiation lineage pathways towards pluripotency, to gain insights into S_{2A}K_{2A}M reprogramming.

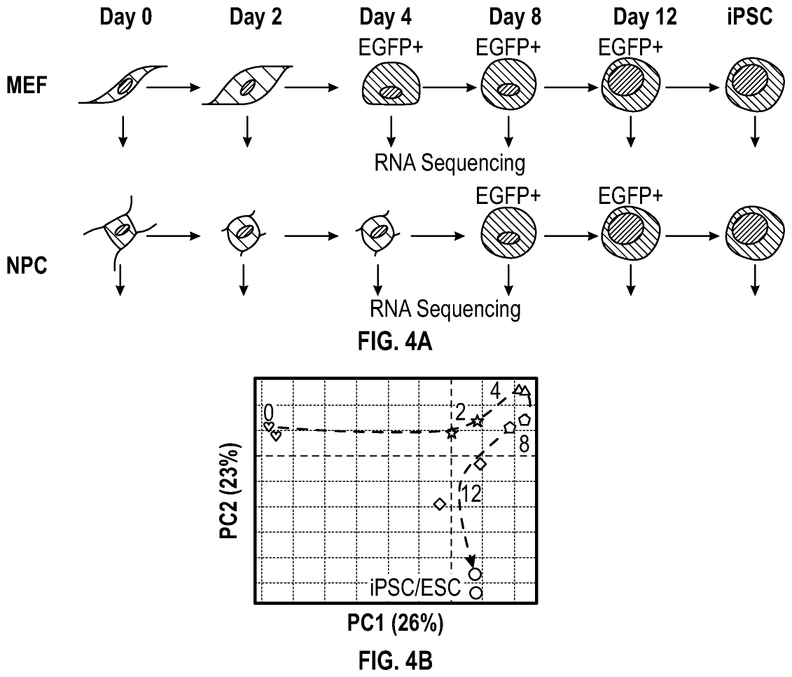

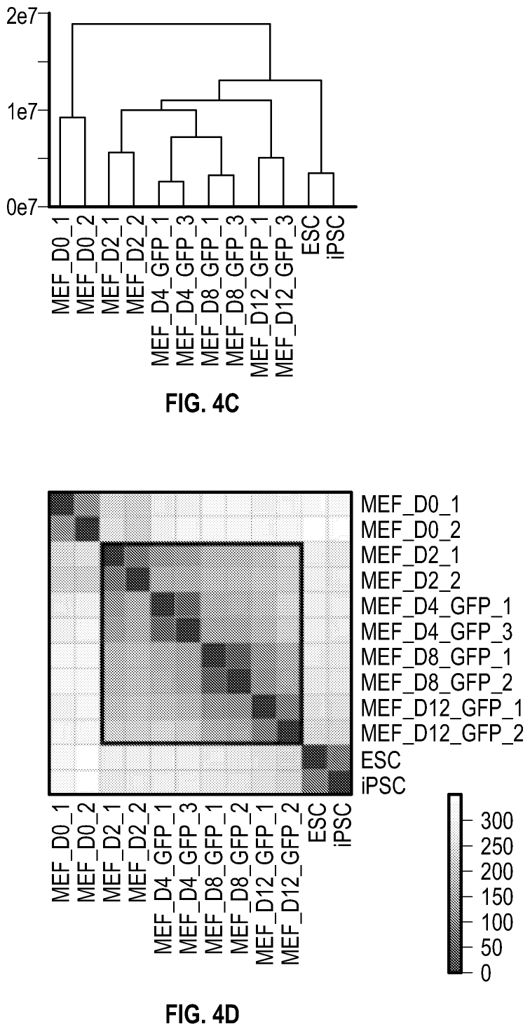

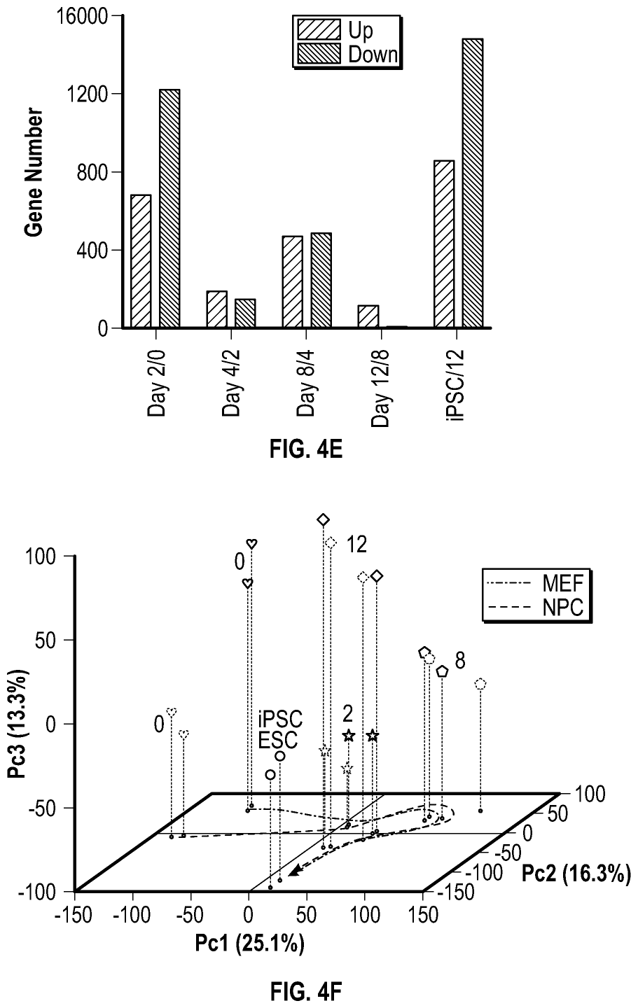

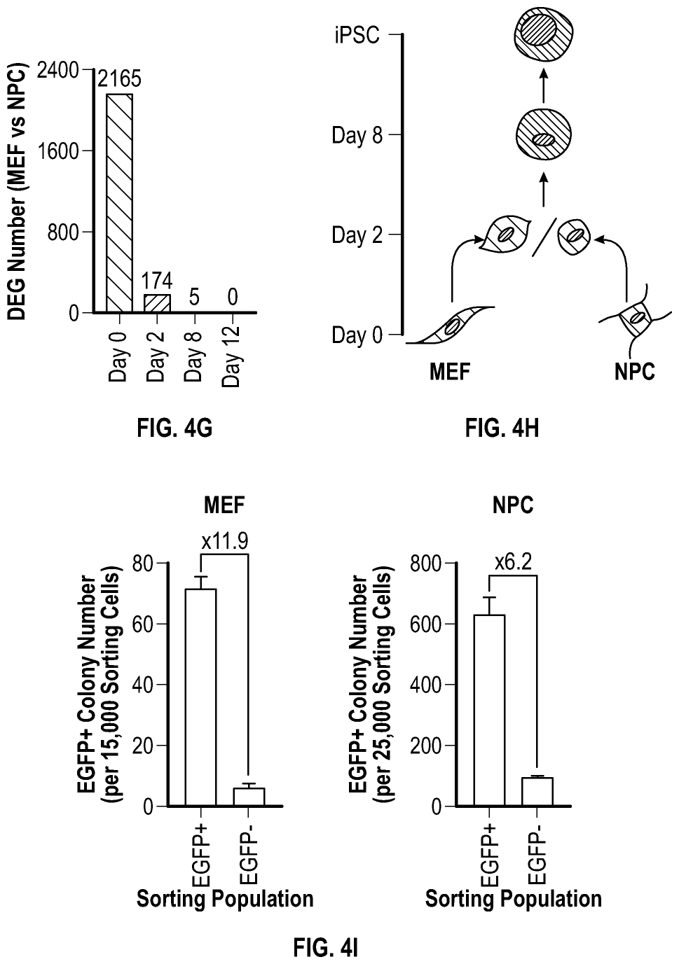

Because of the well-characterized function of Oct4 in pluripotency induction and its early detection in both MEF and NPC reprogramming, the inventors used the activation of endogenous Oct4 to monitor the S_{2A}K_{2A}M reprogramming to pluripotency. As shown in FIG. 4I, EGFP-positive cell populations showed a much higher efficiency for generating iPSC-like colonies than their EGFP-negative counterparts. RNA sequencing (RNA-seq) was performed on cells at days 0, 2, 4, 8, and 12 (FIG. 4A).

Compared to day 0 MEFs, at days 2, 4, 8, and 12 the number of differential expressed genes (DEGs) detected was 1941, 3523, 3910, 2972, and 3969, respectively, in reprogramming intermediates, and iPSCs. FIG. 4B depicts the reprogramming progression from MEFs to iPSCs as provided by principle component analysis (PCA). Cells of the different time points were clearly separated, indicating that these populations were transcriptionally distinct. In particular, the day 2 cells populated away from day 0 MEFs, indicating a robust transcriptional switch within the first 2 days of reprogramming.

Hierarchical clustering placed the reprogramming intermediates from day 2 to day 12 close to each other, indicating that two major transcriptional switches occur between days 0 and 2 (day0/2) and between day 12 and mature iPSCs (day12/iPSC) (FIG. 4C). To verify this, correlation analysis and DEG was used (FIGS. 4D-4E). Larger numbers of DEGs were observed during day0/2 and day12/iPSC transitions, and this was reflected by low correlations between day 0 and day 2 samples as well as between the day 12 samples and iPSCs. These data support the existence of day 0/2 and day 12/iPSC transcriptional switches.

Next, the inventors evaluated whether similar switches occur during NPC reprogramming. Because EGFP-positive cells were not visible on day 4, sorting was only performed on days 8 and 12 (FIG. 4A). The RNA-seq revealed that in the reprogramming NPCs, the number of DEGs was similar to that observed in MEFs at all time points except day 4. Interestingly, day 0/2 and day 12/iPSC transcriptional switches were also identified during NPC reprogramming.

### Example 7: The Molecular Trajectories of MEF and NPC Reprogramming Cells are Convergent

There were 699 upregulated genes during day 0/2 switch in MEF reprogramming. GO analysis revealed the overrepresentation of epithelial genes, indicating mesenchymal-to-epithelial transition (MET) was involved. Interestingly, epithelial genes were also highly enriched in the 880 genes upregulated during the day0/2 switch of NPC reprogramming. This indicates that by day 2, both MEFs and NPCs were reprogrammed towards intermediates with the characteristics of epithelial cells. These analyses indicate that S_{2A}K_{2A}M reprogramming might lead to convergent molecular trajectories after the day 0/2 transcriptional switch in both cell types.

The inventors compared the transcriptional profiles of day 0 MEFs and NPCs. FIG. 4G illustrates that 2165 genes were differentially expressed, of which 1066 and 1099 genes were highly expressed in MEFs and NPCs, respectively. Biological processes related to embryonic fibroblasts were enriched in the MEFs, whereas NPC-enriched genes included those associated with nervous system development, confirming the original identities of the two cell types.

Surprisingly, on day 2, the number of DEGs between reprogramming MEFs and NPCs dropped sharply by 93.8% to 174, indicating the transcriptional similarity of MEF and NPC intermediates. The cell types continued to converge over the course of reprogramming, with no detectable difference in gene expression on day 12 (FIG. 4G).

PCA and correlation analysis clearly supported the disappearance of transcriptional difference between the cell types (FIG. 4F). Starting from day 2, MEF and NPC reprogramming intermediates populated together and were indistinguishable based on the first three principle components, covering 55% of total genes. These data demonstrate that, through dominant activation of similar genes (e.g. the epithelial genes), the molecular trajectories for MEF and NPC reprogramming converge after the day0/2 transcriptional switch (FIG. 4H).

### Example 8: The Day 0/2 Switch Removes Cell Type Identity Markers

This Example describes the major molecular events governing the two transcriptional switches.

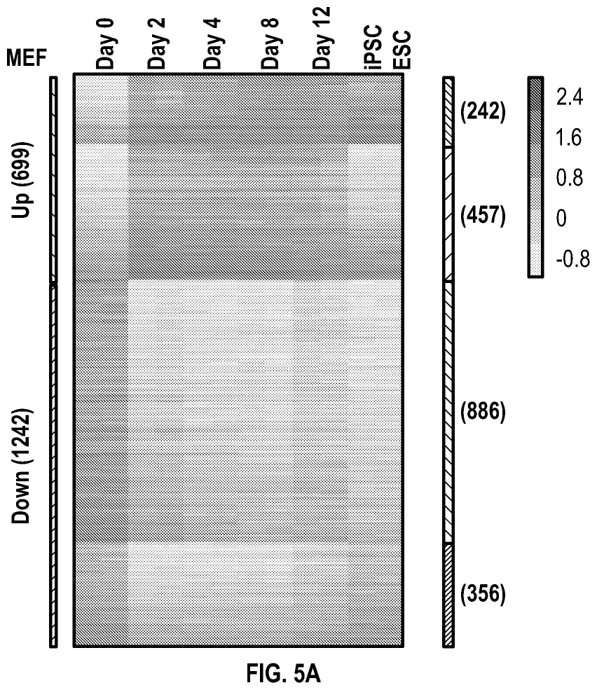

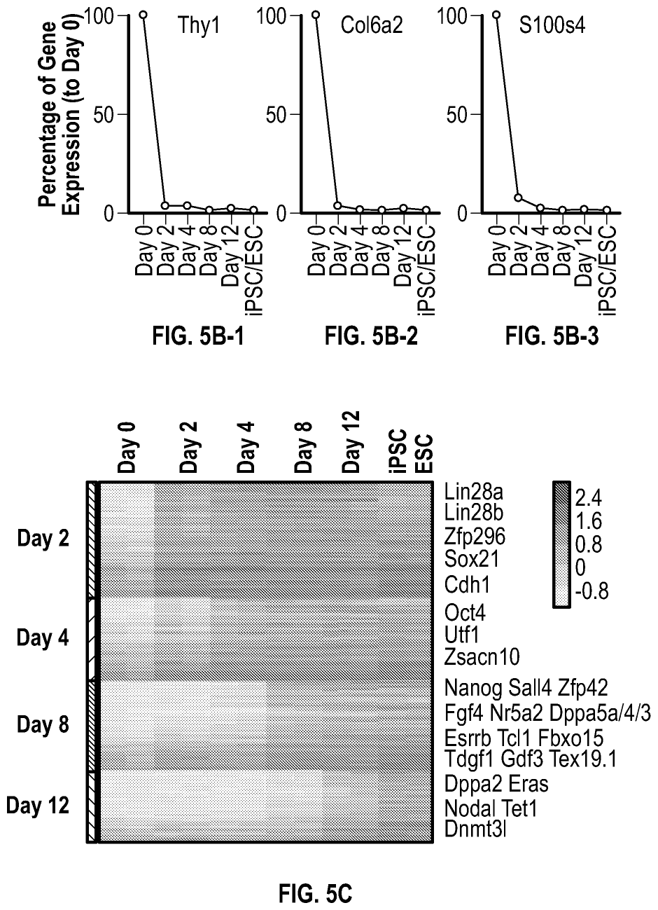

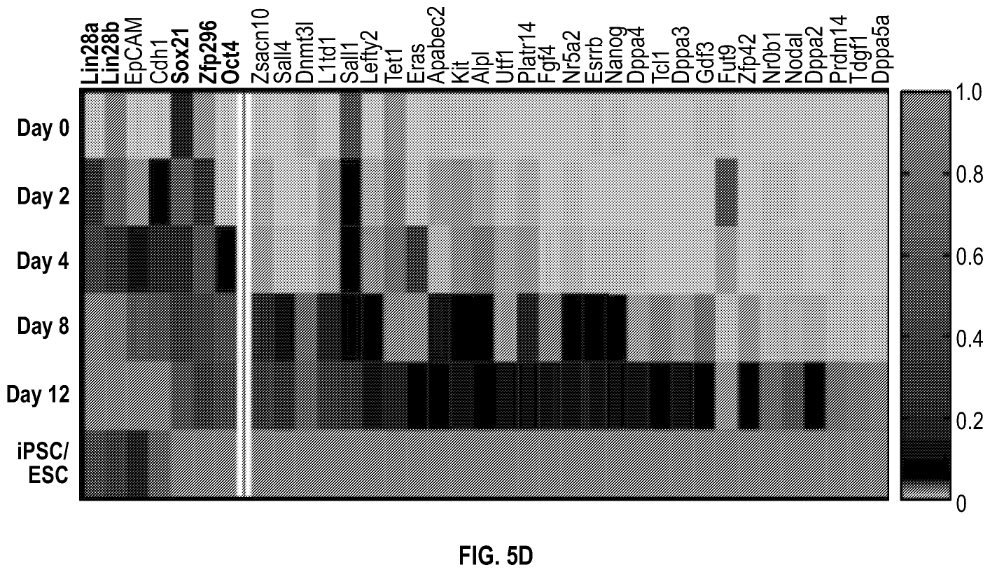

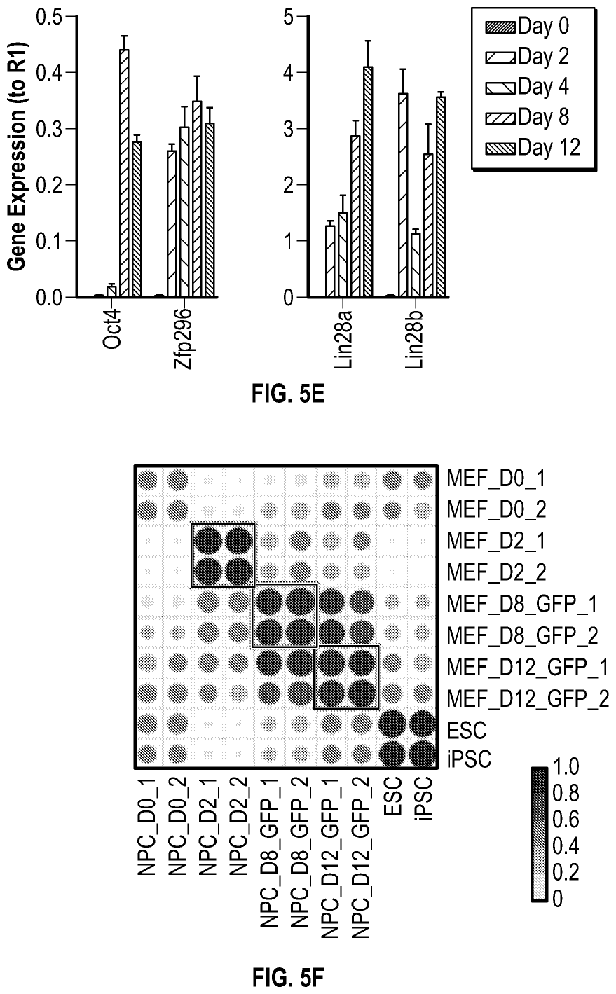

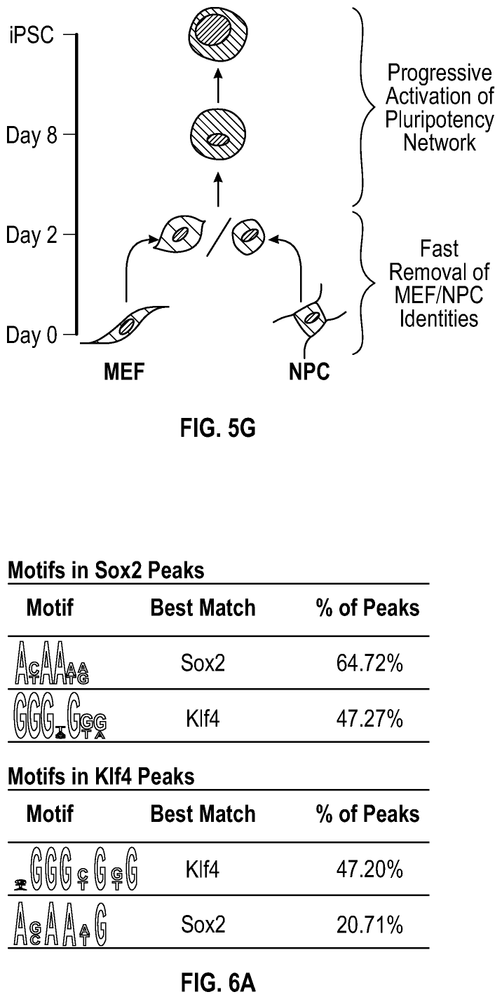

For the day 0/2 switch, many genes were differentially expressed, with 699 upregulated versus 1242 downregulated in MEFs and 880 upregulated versus 1245 downregulated in NPCs (FIG. 5A). Among the downregulated genes, 71.33% (886 out of 1242) and 72.93% (908 out of 1245) were silenced for the rest of reprogramming processes in MEFs and NPCs, respectively, suggesting this inhibition is a critical first step in the induction of pluripotency.

In the MEF gene set, gene ontology (GO) analysis showed that the downregulated genes were mostly responsible for tissue development, and tissue expression analysis revealed enrichment of genes related to fibroblasts and mesenchymal stem cells (Table 3A-3B).

TABLE 3A
GO analysis for the biological processes
of the downregulated 886 genes shown in FIG. 5A

| GO Term | p value |
|---|---|
| system development | 1.20E−43 |
| multicellular organism development | 5.70E−42 |
| anatomical structure morphogenesis | 5.90E−41 |
| blood vessel development | 9.10E−37 |
| regulation of multicellular | 2.40E−34 |
| organismal development | |
| tissue development | 2.60E−34 |
| regulation of cell motility | 2.00E−33 |
| regulation of cellular component movement | 2.30E−33 |

TABLE 3B
Gene enrichment in tissues
with the downregulated 886 genes shown in FIG. 5A.

| Term | p value |
|---|---|
| Fibroblast | 9.50E−09 |
| Mesenchymal Stem Cell | 1.10E−05 |
| Calvaria | 4.30E−05 |
| Macrophage | 2.80E−04 |
| Plasma | 3.90E−04 |
| Bone | 9.00E−04 |
| Cartilage | 1.30E−03 |
| Skin | 2.90E−03 |

These analyses indicated the silencing of MEF program during day 0/2 switch. Downregulation of fibroblast markers was confirmed by qPCR (FIG. 5B).

Similarly, in the NPC reprogramming, the 908 downregulated genes were mainly associated with nervous system development, including Nestin, Lhx2, Nlgn1, et al. Genes expressed in brain, hypothalamus, and cerebellum were overrepresented. Thus, with both MEF and NPC reprogramming, our data indicate that the removal of original cell identities marks the day 0/2 transcriptional switch.

### Example 9: The Pluripotency Network Driving MEF and NPC Reprogramming is Progressively Activated

This Example illustrates how the pluripotency network was established during S_{2A}K_{2A}M reprogramming by showing the expression of pluripotency genes was significantly upregulated.

During MEF reprogramming to iPSCs, 1615 genes were upregulated. These genes were divided into groups based on the timepoint at which they reached a threshold of twofold upregulation, and a pattern of progressive activation of genes was established (FIG. 5C). As shown in FIG. 5D, Lin28a, Lin28b, Zfp296, Sox21, and Cdh1 were upregulated as early as day 2, and by day 4, the expression of another three pluripotent factors, Oct4, Utf1, and Zsacn10, was elevated. These results were confirmed by qPCR analysis (FIG. 5E). By day 8, a larger group of pluripotent factors was elevated, including Nanog, Sall4, Zfp42, Fgf4, Nr5a2, Dppa5/4/3, Esrrb, Tcl1, Tdgf1, Gdf3, Tex19.1, Fbxo15, and by day 12, a few more genes were also activated (e.g., Nodal, Dppa2, Eras, Tet1, and Dnmt3l). These genes showed a flow of gradual activation (FIG. 5D).

A similar analysis was performed of NPC reprogramming. Lin28a, Lin28b, Zfp296, Cdh1, Oct4, Zscan10 were upregulated by day 4. After that, Nanog, Sall4, Tcl1, Fgf4, Zpf42, Gdf3, Utf1, Fbxo15, Esrrb, Dppa4/5, and Nodal were activated on day 8. Fewer genes were found activated by day 12, including Tdgf1, Dppa3, Eras, and Tex19.1. This list was similar to that from MEF reprogramming, with the leading activation of Oct4, Lin28a/b, Zfp296, and Chd1, and a group of other key pluripotent factors following. These observations indicate that independent of the original cell identity, the pluripotency network was gradually established in a similar way during MEF and NPC reprogramming.

To further verify the similar kinetics of pluripotency activation in MEFs and NPCs, 112 pluripotency-related genes were selected, and their expression levels in MEF and NPC reprogramming intermediates were compared in parallel. This correlation analysis revealed that the intermediates at each time points were highly similar (FIG. 5F), suggesting a shared mechanism of pluripotency establishment in MEF and NPC reprogramming (FIG. 5G).

At the day12/iPSC transition, FIG. 5F shows that most key pluripotent genes were further upregulated in MEF and NPC reprogramming at this time point. These data verified that the pluripotency network was stabilized and matured during the day12/iPSC transcriptional switch.

### Example 10: Sox2 and Klf4 Cooperatively Bind and Activate their Targets

This Example illustrates the genome binding patterns of Sox2 and Klf4, which illustrate how S_{2A}K_{2A}M facilitates reprogramming.

Chromatin immunoprecipitation followed by sequencing (ChIP-seq) was performed on day 2 reprogrammed MEFs. Overexpressed proteins tended to bind promiscuously across the genome, so to capture the true binding events, two independent experiments were conducted and only those peaks observed consistently (31236 for Sox2 and 1175 for Klf4) were used in this study. De novo motif discovery showed that Sox2 and Klf4 motifs were highly enriched in the immunoprecipitated DNA fragments, verifying the effectiveness of our experiments (FIG. 6A). Although the genomic distribution of Sox2 and Klf4 binding was similar in reprogramming cells to that in ESCs, there was little overlap between the sites occupied, indicating overexpressed Sox2 and Klf4 could barely access their ESC targets during early reprogramming.

Interestingly, the Klf4 motif was overrepresented in the Sox2 peaks and vice versa (FIG. 6A). The Klf4 motif appeared in about half of Sox2 peaks, whereas Sox2 motif appeared in 20% of Klf4 peaks. As shown in FIGs. The inventors found that hybrid motifs occurred in both Sox2 and Klf4 binding regions which contained at least one Sox2 and one Klf4 motif within 30 base pairs. Furthermore, Sox2 and Klf4 motifs tended to be close to each other (FIG. 6B). Taken together, these data suggest that Sox2 and Klf4 cooperatively bound to their targets. Indeed, the inventors confirmed direct interaction of Sox2 and Klf4 by coimmunoprecipitation (FIG. 6C).

To further investigate their cooperativity, the global colocalization of Sox2 and Klf4 in the genome was analyzed. About 80% of the Klf4 peaks were bound by Sox2 (Sox_Klf peaks) (FIG. 6D). For the peaks called only for Sox2 or Klf4 binding (Sox_solo or Klf_solo peaks), we still observed low level of Klf4 or Sox2 enrichment, respectively (FIG. 6E). This was confirmed by the quantification of the signal intensities (FIG. 6F). This phenomenon demonstrates that Sox2 and Klf4 cooperatively bound their target across the genome with slightly different preference.

The inventors then examined whether this cooperative binding facilitated the activation of their target genes. Sox2 binding (Sox2 Klf and Sox2 solo) led to increased H3K27 acetylation on day 2, but a similar effect was not observed for Klf4 (Klf4_solo) (FIGS. 6E and 6F). This may be because Klf4-bound regions were already highly acetylated. Consistently, the expression of Sox2 target genes was also significantly upregulated by day 2 (FIG. 6G).

### Example 11: Klf4 Overexpression Leads to Sox2 Binding Shift

The inventors then investigated whether Sox2 and Klf4 bindings were the same between the S_{2A}K_{2A}M condition and Sox2 or Klf4 overexpression alone. The samples for Sox2 or Klf4 overexpression alone (Sox2_tetO or Klf4_tetO) were from previous data (Chronis et al., 2017). Although the binding motifs were similar (FIG. 6I), Sox2 binding regions has fundamentally changed in S_{2A}K_{2A}M and Sox2_tetO conditions, with only about 10% overlap (FIG. 6H), while Klf4 binding regions showed high similarity (77% overlap) between S_{2A}K_{2A}M and Klf4_tetO conditions. Because of the overrepresentation of Klf4 motif in the Sox2 binding loci in the S_{2A}K_{2A}M condition, the inventors reasoned that higher Klf4 may be responsible for the Sox2 binding shift. Moreover, the H3K27 acetylation of S_{2A}K_{2A}M-associated peaks (Sox_co and Sox_SKM) was elevated, but the Sox2 peaks specific to the Sox2_tetO condition (Sox tetO) were not (FIG. 6J). However, no binding shift was found for Klf4 peaks.

### Example 12: Sox2 and Klf4 Cooperatively Bind and Activate Pluripotency-Associated Regions

This Example illustrates how Sox2 and Klf4 cooperate in binding and activating pluripotent gene loci. Previously, the inventors had showed that Oct4, Lin28a/b, Zfp296, and Sox21 were upregulated early during MEF reprogramming. In this Example, the inventors investigated whether Sox2 and Klf4 co-occupied these genes.

FIG. 6K shows illustrates that Sox2 and Klf4 binding peaks were observed at the promoters as well as some distal elements near these gene loci, and H3K27 acetylation levels were elevated accordingly.

Because of the critical role of Oct4 in pluripotency induction and maintenance, the inventors studied this case individually with ChIP-qPCR. Primers were designed to cover a large region from the first exon to the distal enhancer of Oct4 along the Oct4 regulatory region of chromosome 17 (FIG. 6K). Similar to the ChIP-seq data, Sox2 and Klf4 binding at the distal enhancer was seen as early as day 2, while much less binding of Sox2 and Klf4 was found on the proximal enhancer and promoter regions (FIG. 6L). These bindings became more pronounced by day 5 (FIG. 6M). Accordingly, the H3K27 acetylation level of this region was dramatically elevated. Thus, before the detection of Oct4 transcription, Sox2 and Klf4 were already bound to the Oct4 locus.

More interestingly, we noticed that co-binding of Sox2 and Klf4 occurs on one of the 231 ESC-specific superenhancers upstream of Oct4. These superenhancers were reported by Whyte and colleagues in 2013, and are associated with the high expression of nearby pluripotent genes (Whyte et al., 2013). The inventors searched whether other ESC-specific super-enhancers were also bound by Sox2 or Klf4. Interestingly, Sox2 binding also occurred on four superenhancers close to Nanog and Sox2, and these superenhancers has been shown to be essential for Nanog and Sox2 expression in ESCs (Blinka et al., 2016; Li et al., 2014; Zhou et al., 2014). A Fgf4 superenhancer was also bound by Sox2. These results demonstrate that on day 2 of S_{2A}K_{2A}M reprogramming, Sox2 and Klf4 cooperatively bound and remodeled some pluripotent gene loci even prior to their transcriptional activation, indicating their function in early priming towards pluripotency.

### REFERENCES

An et al. (2019) Sox2 and Klf4 as the Functional Core in Pluripotency Induction without Exogenous Oct4. Cell Rep 29(7):1986-2000.
Blinka, S., Reimer, M. H., Jr., Pulakanti, K., and Rao, S. (2016). Super-Enhancers at the Nanog Locus Differentially Regulate Neighboring Pluripotency-Associated Genes. Cell Rep 17, 19-28.
Brambrink, T., Foreman, R., Welstead, G. G., Lengner, C. J., Wernig, M., Suh, H., and Jaenisch, R. (2008). Sequential expression of pluripotency markers during direct reprogramming of mouse somatic cells. Cell Stem Cell 2, 151-159.
Carey, B. W., Markoulaki, S., Hanna, J., Saha, K., Gao, Q., Mitalipova, M., and Jaenisch, R. (2009). Reprogramming of murine and human somatic cells using a single polycistronic vector. Proc Natl Acad Sci USA 106, 157-162.
Carey, B. W., Markoulaki, S., Hanna, J. H., Faddah, D. A., Buganim, Y., Kim, J., Ganz, K., Steine, E. J., Cassady, J. P., Creyghton, et al. (2011). Reprogramming factor stoichiometry influences the epigenetic state and biological properties of induced pluripotent stem cells. Cell Stem Cell 9, 588-598.
Chen, J., Chen, X., Li, M., Liu, X., Gao, Y., Kou, X., Zhao, Y., Zheng, W., Zhang, X., Huo, Y., et al. (2016). Hierarchical Oct4 Binding in Concert with Primed Epigenetic Rearrangements during Somatic Cell Reprogramming. Cell Rep 14, 1540-1554.
Chronis, C., Fiziev, P., Papp, B., Butz, S., Bonora, G., Sabri, S., Ernst, J., and Plath, K. (2017). Cooperative Binding of Transcription Factors Orchestrates Reprogramming. Cell 168, 442-459 e420.
de Felipe, P., Luke, G. A., Hughes, L. E., Gani, D., Halpin, C., and Ryan, M. D. (2006). E unum pluribus: multiple proteins from a self-processing polyprotein. Trends Biotechnol 24, 68-75.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Fritz, N. L., Adil, M. M., Mao, S. R., and Schaffer, D. V. (2015). cAMP and EPAC Signaling Functionally Replace OCT4 During Induced Pluripotent Stem Cell Reprogramming. Mol Ther 23, 952-963.
Gao, Y., Chen, J., Li, K., Wu, T., Huang, B., Liu, W., Kou, X., Zhang, Y., Huang, H., Jiang, Y., et al. (2013). Replacement of Oct4 by Tet1 during iPSC induction reveals an important role of DNA methylation and hydroxymethylation in reprogramming. Cell Stem Cell 12, 453-469.
Heng, J. C., Feng, B., Han, J., Jiang, J., Kraus, P., Ng, J. H., Orlov, Y. L., Huss, M., Yang, L., Lufkin, T., et al. (2010). The nuclear receptor Nr5a2 can replace Oct4 in the reprogramming of murine somatic cells to pluripotent cells. Cell Stem Cell 6, 167-174.
Hockemeyer, D., Soldner, F., Cook, E. G., Gao, Q., Mitalipova, M., and Jaenisch, R. (2008). A Drug-Inducible System for Direct Reprogramming of Human Somatic Cells to Pluripotency. Cell Stem Cell 3, 346-353.
Kim, J. B., Greber, B., Arauzo-Bravo, M. J., Meyer, J., Park, K. I., Zaehres, H., and Scholer, H. R. (2009a). Direct reprogramming of human neural stem cells by OCT4. Nature 461, 649-643.
Kim, J. B., Sebastiano, V., Wu, G., Arauzo-Bravo, M. J., Sasse, P., Gentile, L., K Ruau, D., Ehrich, M., van den Boom, D., et al. (2009b). Oct4-induced pluripotency in adult neural stem cells. Cell 136, 411-419.
Kim, S. I., Oceguera-Yanez, F., Hirohata, R., Linker, S., Okita, K., Yamada, Y., Yamamoto, T., Yamanaka, S., and Woltjen, K. (2015). KLF4 N-terminal variance modulates induced reprogramming to pluripotency. Stem Cell Reports 4, 727-743.
Langmead, B., and Salzberg, S. L. (2012). Fast gapped-read alignment with Bowtie 2. Nat Methods 9, 357-359.
Langmead, B., Trapnell, C., Pop, M., and Salzberg, S. L. (2009). Ultrafast and memory-efficient alignment of short DNA sequences to the human genome. Genome Biol 10, R25.
Li, Y., Rivera, C. M., Ishii, H., Jin, F., Selvaraj, S., Lee, A. Y., Dixon, J. R., and Ren, B. (2014). CRISPR reveals a distal super-enhancer required for Sox2 expression in mouse embryonic stem cells. PLoS One 9, e114485.
Lin, C. Y., Loven, J., Rahl, P. B., Paranal, R. M., Burge, C. B., Bradner, J. E., Lee, T. I., and Young, R. A. (2012). Transcriptional amplification in tumor cells with elevated c-Myc. Cell 151, 56-67.
Liu, P., Chen, M., Liu, Y., Qi, L. S., and Ding, S. (2018). CRISPR-Based Chromatin Remodeling of the Endogenous Oct4 or Sox2 Locus Enables Reprogramming to Pluripotency. Cell Stem Cell 22, 252-261 e254.
Love, M. I., Huber, W., and Anders, S. (2014). Moderated estimation of fold change and dispersion for RNA-seq data with DESeq2. Genome Biol 15, 550.
Machanick, P., and. Bailey, T. L. (2011). MFMF-ChIP: motif analysis of large DNA datasets. Bioinformatics 27, 1696-1697.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat Biotechnol 28, 495-501.
Meisner, L. F., and Johnson, J. A. (2008). Protocols for cytogenetic studies of human embryonic stem cells. Methods 45, 133-141.
Nakagawa, M., Koyanagi, M., Tanabe, K., Takahashi, K., Ichisaka, T., Aoi, T., Okita, K., Mochiduki, Y., Takizawa, N., and Yamanaka, S. (2008). Generation of induced pluripotent stem cells without Myc from mouse and human fibroblasts. Nat Biotechnol 26, 101-106.
Nefzger, C. M., Rossello, F. J., Chen, J., Liu, X., Knaupp, A. S., Firas, J., Paynter, Pflueger, J., Buckberry, S., Lim, S. M., et al. (2017). Cell Type of Origin Dictates the Route to Pluripotency. Cell Rep 21, 2649-2660.
Nie, Z., Hu, G., Wei, G., Cui, K., Yamane, A., Resch, W., Wang, R., Green, D. R., Tessarollo, L., Casellas, R., et al. (2012). c-Myc is a universal amplifier of expressed genes in lymphocytes and embryonic stem cells. Cell 151, 68-79.
Papapetrou, E. P., Tomishima, M. J., Chambers, S. M., Mica, Y., Reed, E., Menon, J., Tabar, V., Mo, Q., Studer, L., and Sadelain, M. (2009). Stoichiometric and temporal requirements of Oct4, Sox2, Klf4, and c-Myc expression for efficient human iPSC induction and differentiation. Proc Natl Acad Sci USA 106, 12759-12764.
Polo, J. M., Anderssen, E., Walsh, R. M., Schwarz, B. A., Nefzger, C. M., Lim, S. M., Borkent, M., Apostolou, E., Alaei, S., Cloutier, J., et al. (2012). A molecular roadmap of reprogramming somatic cells into iPS cells. Cell 151, 1617-1632.
Quinlan, A. R., and Hall, I. M. (2010). BEDTools: a flexible suite of utilities for comparing genomic features. Bioinformatics 26, 841-842.
Redmer, T., Diecke, S., Grigoryan, T., Quiroga-Negreira, A., Birchmeier, W., and Besser, D. (2011). E-cadherin is crucial for embryonic stem cell pluripotency and can replace OCT4 during somatic cell reprogramming. EMBO Rep 12, 720-726.
Robinson, J. T., Thorvaldsdottir, H., Winckler, W., Guttman, M., Lander, E. S., Getz, G., and Mesirov, J. P. (2011). Integrative genomics viewer. Nat Biotechnol 29, 24-26.
Shen, L., Shao, N., Liu, X., and Nestler, E. (2014). ngs.plot: Quick mining and visualization of next-generation sequencing data by integrating genomic databases. BMC Genomics 15, 284.
Shu, J., Wu, C., Wu, Y., Li, Z., Shao, S., Zhao, W., Tang, X., Yang, H., Shen, L., Zuo, X., et al. (2013). Induction of pluripotency in mouse somatic cells with lineage specifiers. Cell 153, 963-975.
Smith, Z. D., Sindhu, C., and Meissner, A. (2016). Molecular features of cellular reprogramming and development. Nat Rev Mol Cell Biol 17, 139-154.
Soufi, A., Donahue, G., and Zaret, K. S. (2012). Facilitators and impediments of the pluripotency reprogramming factors' initial engagement with the genome. Cell 151, 994-1004.
Sridharan, R., Tchieu, J., Mason, M. J., Yachechko, R., Kuoy, E., Horvath, S., Zhou, Q., and Plath, K. (2009). Role of the murine reprogramming factors in the induction of pluripotency. Cell 136, 364-377.
Szabo, P. E., Hubner, K., Scholer, H., and Mann, J. R. (2002). Allele-specific expression of imprinted genes in mouse migratory primordial germ cells. Mech Dev 115, 157-160.
Takahashi, K., and. Yamanaka, S. (2006). Induction of pluripotent stem cells from mouse embryonic and adult fibroblast cultures by defined factors. Cell 126, 663-676.
Tan, F., Qian, C., Tang, K., Abd-Allah, S. M., and Jing, N. (2015). Inhibition of transforming growth factor beta (TGF-beta) signaling can substitute for Oct4 protein in reprogramming and maintain pluripotency. J Biol Chem 290, 4500-4511.
Tiemann, U., Sgodda, M., Warlich, E., Ballmaier, M., Scholer, H. R., Schambach, A., and Cantz, T. (2011). Optimal reprogramming factor stoichiometry increases colony numbers and affects molecular characteristics of murine induced pluripotent stem cells. Cytometry A 79, 426-435.
Wernig, M., Lengner, C. J., Hanna, J., Lodato, Steine, E., Foreman, R., Staerk, J., Markoulaki, S., and Jaenisch, R. (2008). A drug-inducible transgenic system for direct reprogramming of multiple somatic cell types. Nat Biotechnol 26, 916-924.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, 8137.
Zhou, H. Y., Katsman, Y., Dhaliwal, N. K., Davidson, S., Macpherson, N. N., Sakthidevi, M., Collura, F., and Mitchell, J. A. (2014). A Sox2 distal enhancer cluster regulates embryonic stem cell differentiation potential. Genes Dev 28, 2699-2711.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. A polycistronic expression cassette comprising a promoter operably linked to a nucleic acid segment encoding a Sox2 polypeptide, Klf4 polypeptide, and optionally a c-Myc polypeptide.
2. The polycistronic expression cassette of statement 1, wherein the nucleic acid segment encodes a Sox2 polypeptide in frame with a Klf4 polypeptide, and optionally in frame with a c-Myc polypeptide, as a single continuous opening reading frame.
3. The polycistronic expression cassette of statement 1 or 2, wherein the nucleic acid segment further encodes one or more cleavable peptide linkers between the Sox2 polypeptide, the Klf4 polypeptide, and/or the optional c-Myc polypeptide.
4. The polycistronic expression cassette of statement 1, 2 or 3, wherein the promoter is heterologous to the nucleic segment encoding the Sox2 polypeptide, the Klf4 polypeptide, and the optional Myc polypeptide.
5. The polycistronic expression cassette of statement 1-3 or 4, wherein the promoter is an inducible promoter.
6. The polycistronic expression cassette of statement 1-3 or 4, wherein the promoter is a constitutive promoter.
7. A host cell comprising the polycistronic expression cassette of statement 1-5 or 6.
8. The host cell of statement 7, which is an adult cell.
9. The host cell of statement 7 or 8, which is autologous to a selected patient or animal.
10. The host cell of statement 9, wherein the animal is an experimental (e.g., lab) animal, a domesticated animal, an endangered animal, or a zoo animal.
11. The host cell of statement 9 or 10, wherein the selected patient has a disease or medical condition.
12. The host cell of statement 7-10 or 11, which is within a population of cells.
13. A method comprising contacting a selected cell with the polycistronic expression cassette of statement 1-4 or 5 to thereby generate a host cell that comprises the polycistronic expression cassette.
14. The method of statement 13, further comprising incubating the host cell in reprogramming medium to generate a reprogrammed cell.
15. The method of statement 13 or 14, wherein incubating the host cell in reprogramming medium reprograms the host cell to cross cellular lineage boundaries so that the reprogrammed cell has a different phenotype than the host cell.
16. The method of statement 14 or 15, wherein the reprogramming medium does not have Chirr99021, PD0325901, or a combination of Chirr99021 and PD0325901.
17. The method of statement 14, 15 or 16, wherein the reprogramming medium comprises an inducing agent.
18. The method of statement 14-16 or 17, wherein the reprogramming medium comprises doxycycline.
19. The method of statement 14-17 or 18, wherein the reprogramming medium comprises doxycycline, A83-01, Forskolin, or a combination thereof
20. The method of statement 14-18 or 19, further comprising incubating the reprogrammed cell in a culture medium for a time sufficient to generate a population of reprogrammed cells.
21. The method of statement 14-19 or 20, wherein a population of host cells are incubated with the reprogramming medium.
22. The method of statement 21, wherein at least 1%, at least 3%, at least 5%, at least 6%, at least 7%, at least 8%, at least 9%, at least 10%, at least 11%, at least 12%, at least 13%, at least 14%, or at least 15% of the population of host cells are reprogrammed as reprogrammed cells.
23. The method of statement 14-21 or 22, wherein the reprogrammed cell or the reprogrammed cells is/are a stem cell(s).
24. The method of statement 14-22 or 23, wherein the reprogrammed cell(s) is/are pluripotent stem cell(s).
25. The method of statement 23 or 24, further comprising differentiating the stem cell(s) or pluripotent stem cell(s) into ectodermal cell(s), a mesodermal cell(s), or endodermal cell(s).
26. The method of statement 23, 24 or 25, further comprising differentiating the stem cell(s) or pluripotent stem cell(s) into neuronal cell(s), cardiomyocyte(s), pancreatic cell(s), hepatic cell(s), dermal cell(s), chondrocyte(s), or progenitors thereof
27. The method of statement 24, further comprising generating an animal embryo from the pluripotent stem cell(s).
28. The method of statement 14-24 or 25, further comprising administering the reprogrammed cell(s) or the stem cell(s) or the cell(s) to a patient or an animal.
29. The method of statement 26, further comprising administering to a patient or an animal the neuronal cell(s), cardiomyocyte(s), pancreatic cell(s), hepatic cell(s), dermal cell(s), chondrocyte(s), or progenitors thereof.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed:

1. A polycistronic expression cassette comprising a promoter operably linked to a nucleic acid segment encoding a Sox2 polypeptide in frame with a Klf4 polypeptide, and optionally a c-Myc polypeptide in frame therewith, as a single continuous opening reading frame.

2. The polycistronic expression cassette of claim 1, wherein the nucleic acid segment encodes a Sox2 polypeptide, a Klf4 polypeptide, and c-Myc polypeptide.

3. The polycistronic expression cassette of claim 1, wherein the nucleic acid segment further encodes one or more cleavable peptide linkers between the Sox2 polypeptide and the Klf4 polypeptide.

4. The polycistronic expression cassette of claim 1, wherein the nucleic acid segment further encodes a cleavable peptide linker adjoining the c-Myc polypeptide coding region to the opening reading frame.

5. The polycistronic expression cassette of claim 1, wherein the promoter is heterologous to the nucleic segment encoding the Sox2 polypeptide and the Klf4 polypeptide.

6. The polycistronic expression cassette of claim 1, wherein the promoter is an inducible promoter.

7. The polycistronic expression cassette of claim 1 which is within a vector.

8. The polycistronic expression cassette of claim 7, wherein the vector is a lentiviral vector, adenoviral vector, adeno-associated viral vector, herpes viral vector, vaccinia viral vector, polio viral vector, AIDS viral vector, neuronal trophic viral vector, or Sindbis viral vector.

9. A host cell comprising a polycistronic expression cassette comprising a promoter operably linked to a nucleic acid segment encoding a Sox2 polypeptide in frame with a Klf4 polypeptide, and optionally encoding a c-Myc polypeptide in frame therewith, as a single continuous opening reading frame.

10. The host cell of claim 9, wherein the polycistronic expression cassette is within a vector.

11. The host cell of claim 9, wherein the polycistronic expression cassette is integrated into the host cell genome.

12. The host cell of claim 9, wherein the polycistronic expression vectors is maintained episomally in the host cell.

13. The host cell of claim 9, which is an adult cell.

14. The host cell of claim 9, which is autologous to a selected patient or animal.

15. The host cell of claim 14, which has a mutation correlated with a disease or condition.

16. A method comprising contacting a selected cell with a polycistronic expression cassette comprising a promoter operably linked to a nucleic acid segment encoding a Sox2 polypeptide in frame with a Klf4 polypeptide, and optionally encoding a c-Myc polypeptide in frame therewith, as a single continuous opening reading frame, to thereby generate a host cell that comprises the polycistronic expression cassette.

17. The method of claim 16, further comprising incubating the host cell in reprogramming medium to generate a reprogrammed pluripotent stem cell.

18. The method of claim 17, wherein the reprogramming medium comprises Forskolin and A83-01.

19. The method of claim 16, wherein the nucleic acid segment encodes a Sox2 polypeptide, a Klf4 polypeptide, and c-Myc polypeptide.

20. The method of claim 16, wherein the nucleic acid segment further encodes one or more cleavable peptide linkers between the Sox2 polypeptide and the Klf4 polypeptide.

21. The method of claim 20, wherein the nucleic acid segment further encodes a cleavable peptide linker adjoining the c-Myc polypeptide coding region to the opening reading frame.

22. The method of claim 16, wherein the promoter is heterologous to the nucleic segment encoding the Sox2 polypeptide and the Klf4 polypeptide.

23. The method of claim 16, wherein the promoter is an inducible promoter.

24. The method of claim 17, further comprising differentiating the reprogrammed pluripotent stem cell into a differentiated cell.


SEQUENCE LISTING (95 sequences)

SEQ ID NO: 1 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 2 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 3 - protein, 321 aa, Homo sapiens
HSARMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 4 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 6 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 7 - skipped

SEQ ID NO: 8 - protein, 22 aa, Artificial Sequence
  A synthetic amino acid sequence
GSGATNFSLLKQAGDVEENPGP

SEQ ID NO: 9 - protein, 21 aa, Artificial Sequence
  A synthetic amino acid sequence
GSGEGRGSLLTCGDVEENPGP

SEQ ID NO: 10 - protein, 23 aa, Artificial Sequence
  A synthetic amino acid sequence
GSGQCTNYALLKLAGDVESNPGP

SEQ ID NO: 11 - protein, 25 aa, Artificial Sequence
  A synthetic amino acid sequence
GSGVKQTLNFDLLKLAGDVESNPGP

SEQ ID NO: 12 - protein, 9 aa, Artificial Sequence
  A synthetic amino acid sequence
SNAADEVAT

SEQ ID NO: 13 - protein, 29 aa, Artificial Sequence
  A synthetic amino acid sequence
SNAADEVATQLLNFDLLKLAGDVESNPGP

SEQ ID NO: 14 - protein, 24 aa, Artificial Sequence
  A synthetic amino acid sequence
APVKQLLNFDLLKLAGDVESNPGP

SEQ ID NO: 15 - protein, 24 aa, Artificial Sequence
  A synthetic amino acid sequence
SGSGQLLNFDLLKLAGDVESNPGP

SEQ ID NO: 16 - protein, 1309 aa, Artificial Sequence
  A synthetic amino acid sequence
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHMACGSGEGRGSLLTCGDVEENPGPLEMRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLEGSGQCTNYALLKLAGDVESNPGPGAPLDFLWALETPQTATTMPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 17 - protein, 827 aa, Artificial Sequence
  A synthetic amino acid sequence
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHMACGSGEGRGSLLTCGDVEENPGPLEMRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 18 - DNA, 43 bp, Artificial Sequence
  A synthetic oligonucleotide sequence
gaccgatccagcctccgcggccccggccatggctggacacctg

SEQ ID NO: 19 - DNA, 34 bp, Artificial Sequence
  A synthetic oligonucleotide sequence
cgttgaggggcatctcgagtgggccgggattttc

SEQ ID NO: 20 - DNA, 36 bp, Artificial Sequence
  A synthetic oligonucleotide sequence
cggcccactcgagatgcccctcaacgtgaacttcac

SEQ ID NO: 21 - DNA, 41 bp, Artificial Sequence
  A synthetic oligonucleotide sequence
ttgattatcgataagcttgatatcgggcgcgccttatgcac

SEQ ID NO: 22 - DNA, 24 bp, Artificial Sequence
  A synthetic oligonucleotide sequence
gcagctagctgcatggctggacac

SEQ ID NO: 23 - DNA, 24 bp, Artificial Sequence
  A synthetic oligonucleotide sequence
gcagaattcggcgcgccttatgca

SEQ ID NO: 24 - DNA, 24 bp, Artificial Sequence
  A synthetic oligonucleotide sequence
gcagaattctgcatgtataacatg

SEQ ID NO: 25 - DNA, 24 bp, Artificial Sequence
  A synthetic oligonucleotide sequence
gcagaattcggcgcgccttatgca

SEQ ID NO: 26 - DNA, 24 bp, Artificial Sequence
  A synthetic oligonucleotide sequence
gcagaattctgcatgtataacatg

SEQ ID NO: 27 - DNA, 24 bp, Artificial Sequence
  A synthetic oligonucleotide sequence
gcagaattcttaaaagtgcctctt

SEQ ID NO: 28 - DNA, 33 bp, Artificial Sequence
  A synthetic oligonucleotide sequence
gcctccgcggccccgaattcgccatgaggcagc

SEQ ID NO: 29 - DNA, 26 bp, Artificial Sequence
  A synthetic oligonucleotide sequence
ccgctagctgcaccagagtttcgaag

SEQ ID NO: 30 - DNA, 26 bp, Artificial Sequence
  A synthetic oligonucleotide sequence
ctggtgcagctagcggcagcggcgcc

SEQ ID NO: 31 - DNA, 49 bp, Artificial Sequence
  A synthetic oligonucleotide sequence
cgataagcttgatatcgaattcggcgcgcctcacatgtgcgacaggggc

SEQ ID NO: 32 - DNA, 33 bp, Artificial Sequence
  A synthetic oligonucleotide sequence
gcctccgcggccccgaattcgccatgcccctca

SEQ ID NO: 33 - DNA, 27 bp, Artificial Sequence
  A synthetic oligonucleotide sequence
ccgctagctgcaccagagtttcgaagc

SEQ ID NO: 34 - DNA, 26 bp, Artificial Sequence
  A synthetic oligonucleotide sequence
ctggtgcagctagcggcagcggcgcc

SEQ ID NO: 35 - DNA, 43 bp, Artificial Sequence
  A synthetic oligonucleotide sequence
cgataagcttgatatcgaattcggcgcgccttaaaagtgcctc

SEQ ID NO: 36 - DNA, 19 bp, Artificial Sequence
  A synthetic oligonucleotide sequence
acatcgccaatcagcttgg

SEQ ID NO: 37 - DNA, 23 bp, Artificial Sequence
  A synthetic oligonucleotide sequence
agaaccatactcgaaccacatcc

SEQ ID NO: 38 - DNA, 20 bp, Artificial Sequence
  A synthetic oligonucleotide sequence
acagatgcaaccgatgcacc

SEQ ID NO: 39 - DNA, 20 bp, Artificial Sequence
  A synthetic oligonucleotide sequence
tggagttgtactgcagggcg

SEQ ID NO: 40 - DNA, 20 bp, Artificial Sequence
  A synthetic oligonucleotide sequence
gcacacctgcgaactcacac

SEQ ID NO: 41 - DNA, 21 bp, Artificial Sequence
  A synthetic oligonucleotide sequence
ccgtcccagtcacagtggtaa

SEQ ID NO: 42 - DNA, 19 bp, Artificial Sequence
  A synthetic oligonucleotide sequence
ccaccagcagcgactctga

SEQ ID NO: 43 - DNA, 21 bp, Artificial Sequence
  A synthetic oligonucleotide sequence
tgcctcttctccacagacacc

SEQ ID NO: 44 - DNA, 20 bp, Artificial Sequence
  A synthetic oligonucleotide sequence
atgggaaggaagggacaatc

SEQ ID NO: 45 - DNA, 20 bp, Artificial Sequence
  A synthetic oligonucleotide sequence
atacaaactcccgctgatcg

SEQ ID NO: 46 - DNA, 22 bp, Artificial Sequence
  A synthetic oligonucleotide sequence
cctccagcagatgcaagaactc

SEQ ID NO: 47 - DNA, 23 bp, Artificial Sequence
  A synthetic oligonucleotide sequence
cttcaaccactggtttttctgcc

SEQ ID NO: 48 - DNA, 20 bp, Artificial Sequence
  A synthetic oligonucleotide sequence
ctcgccaactcagattcgat

SEQ ID NO: 49 - DNA, 20 bp, Artificial Sequence
  A synthetic oligonucleotide sequence
agaagtgttgcacggctttg

SEQ ID NO: 50 - DNA, 23 bp, Artificial Sequence
  A synthetic oligonucleotide sequence
cgtggtgagcatcttcggagtgg

SEQ ID NO: 51 - DNA, 23 bp, Artificial Sequence
  A synthetic oligonucleotide sequence
ccttcttggtccgcccgttctta

SEQ ID NO: 52 - DNA, 22 bp, Artificial Sequence
  A synthetic oligonucleotide sequence
tctcactcatgttgcgggaccc

SEQ ID NO: 53 - DNA, 22 bp, Artificial Sequence
  A synthetic oligonucleotide sequence
cgtcggagttgaaatgggcgaa

SEQ ID NO: 54 - DNA, 19 bp, Artificial Sequence
  A synthetic oligonucleotide sequence
tgtcccggtgactacgtct

SEQ ID NO: 55 - DNA, 21 bp, Artificial Sequence
  A synthetic oligonucleotide sequence
cccagaagtagctccgtctct

SEQ ID NO: 56 - DNA, 20 bp, Artificial Sequence
  A synthetic oligonucleotide sequence
tatgactcacttccaggggg

SEQ ID NO: 57 - DNA, 20 bp, Artificial Sequence
  A synthetic oligonucleotide sequence
agaagaaagcaggatcgcct

SEQ ID NO: 58 - DNA, 19 bp, Artificial Sequence
  A synthetic oligonucleotide sequence
atggaggggaatacagccc

SEQ ID NO: 59 - DNA, 20 bp, Artificial Sequence
  A synthetic oligonucleotide sequence
ttctttgcagctccttcgtt

SEQ ID NO: 60 - DNA, 21 bp, Artificial Sequence
  A synthetic oligonucleotide sequence
tcatcgctttcatggatcaca

SEQ ID NO: 61 - DNA, 21 bp, Artificial Sequence
  A synthetic oligonucleotide sequence
acagcaacttccaaggactag

SEQ ID NO: 62 - DNA, 19 bp, Artificial Sequence
  A synthetic oligonucleotide sequence
gaagagatccacagccctg

SEQ ID NO: 63 - DNA, 23 bp, Artificial Sequence
  A synthetic oligonucleotide sequence
ccaaagaataaccctgactcctg

SEQ ID NO: 64 - DNA, 21 bp, Artificial Sequence
  A synthetic oligonucleotide sequence
gagtcaatacgggtaacaggc

SEQ ID NO: 65 - DNA, 19 bp, Artificial Sequence
  A synthetic oligonucleotide sequence
ttctcgcacagtccacatc

SEQ ID NO: 66 - DNA, 24 bp, Artificial Sequence
  A synthetic oligonucleotide sequence
aacaactgcatgaaggcgggaatc

SEQ ID NO: 67 - DNA, 24 bp, Artificial Sequence
  A synthetic oligonucleotide sequence
cctgtgcagctggctcaaatcaaa

SEQ ID NO: 68 - DNA, 24 bp, Artificial Sequence
  A synthetic oligonucleotide sequence
gctggcaacaagttgctctctgaa

SEQ ID NO: 69 - DNA, 24 bp, Artificial Sequence
  A synthetic oligonucleotide sequence
cgttgcactgcttggctttgaaga

SEQ ID NO: 70 - DNA, 21 bp, Artificial Sequence
  A synthetic oligonucleotide sequence
tccatcagggtgactcagaaa

SEQ ID NO: 71 - DNA, 19 bp, Artificial Sequence
  A synthetic oligonucleotide sequence
ccagcttcaaggggctcaa

SEQ ID NO: 72 - DNA, 24 bp, Artificial Sequence
  A synthetic oligonucleotide sequence
cctccaatggcaaagtgaatggca

SEQ ID NO: 73 - DNA, 24 bp, Artificial Sequence
  A synthetic oligonucleotide sequence
tgtttcatagtggtcagggtccgt

SEQ ID NO: 74 - DNA, 20 bp, Artificial Sequence
  A synthetic oligonucleotide sequence
tgaatgagtgatgtcgtggg

SEQ ID NO: 75 - DNA, 21 bp, Artificial Sequence
  A synthetic oligonucleotide sequence
cttctgatcctcttgccttcc

SEQ ID NO: 76 - DNA, 20 bp, Artificial Sequence
  A synthetic oligonucleotide sequence
catgtcgctgaaactcctca

SEQ ID NO: 77 - DNA, 20 bp, Artificial Sequence
  A synthetic oligonucleotide sequence
aatggactcacggaggacac

SEQ ID NO: 78 - DNA, 20 bp, Artificial Sequence
  A synthetic oligonucleotide sequence
tggcctggaactcagaaatc

SEQ ID NO: 79 - DNA, 20 bp, Artificial Sequence
  A synthetic oligonucleotide sequence
tgcctcctgggtcttagaaa

SEQ ID NO: 80 - DNA, 20 bp, Artificial Sequence
  A synthetic oligonucleotide sequence
gacggcagatgcataacaaa

SEQ ID NO: 81 - DNA, 20 bp, Artificial Sequence
  A synthetic oligonucleotide sequence
aaggaagggctaggacgaga

SEQ ID NO: 82 - DNA, 20 bp, Artificial Sequence
  A synthetic oligonucleotide sequence
cccaggctcagaactctgtc

SEQ ID NO: 83 - DNA, 20 bp, Artificial Sequence
  A synthetic oligonucleotide sequence
tgctcctacaccatgctctg

SEQ ID NO: 84 - DNA, 20 bp, Artificial Sequence
  A synthetic oligonucleotide sequence
tcctcctaatcccgtctcct

SEQ ID NO: 85 - DNA, 20 bp, Artificial Sequence
  A synthetic oligonucleotide sequence
ataccctgcttcccttcctc

SEQ ID NO: 86 - DNA, 20 bp, Artificial Sequence
  A synthetic oligonucleotide sequence
ctggggacatatctggttgg

SEQ ID NO: 87 - DNA, 20 bp, Artificial Sequence
  A synthetic oligonucleotide sequence
cccagtatttcagcccatgt

SEQ ID NO: 88 - DNA, 20 bp, Artificial Sequence
  A synthetic oligonucleotide sequence
ttgaaaatgaaggcctcctg

SEQ ID NO: 89 - DNA, 20 bp, Artificial Sequence
  A synthetic oligonucleotide sequence
agcgctatctgcctgtgtct

SEQ ID NO: 90 - DNA, 20 bp, Artificial Sequence
  A synthetic oligonucleotide sequence
taggtgagccgtctttccac

SEQ ID NO: 91 - DNA, 20 bp, Artificial Sequence
  A synthetic oligonucleotide sequence
gcttagccaggttcgaggat

SEQ ID NO: 92 - DNA, 25 bp, Artificial Sequence
  A synthetic oligonucleotide sequence
gaggattggaggtgtaatggttgtt

SEQ ID NO: 93 - DNA, 22 bp, Artificial Sequence
  A synthetic oligonucleotide sequence
ctactacccatcacccccacta

SEQ ID NO: 94 - DNA, 31 bp, Artificial Sequence
  A synthetic oligonucleotide sequence
caagctttgggttgaaatattgggtttattt

SEQ ID NO: 95 - DNA, 30 bp, Artificial Sequence
  A synthetic oligonucleotide sequence
cggatccctaaaaccaaatatccaaccata